(19) United States
(12) Patent Application Publication
(10) Pub. No.: US 2003/0022851 A1
(43) Pub. Date: Jan. 30, 2003

(54) Long term memory, synaptic plasticity and the ADF1 transcription factor

(75) Inventors: Timothy P. Tully, Cold Spring Harbor, NY (US); James D. DeZazzo, Ann Arbor, MI (US); Mani Ramaswami, Tucson, AZ (US); David J. Sandstrom, Chevy Chase, MD (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Cold Spring Harbor Laboratory, Cold Spring Harbor, NY (US)

(21) Appl. No.: 09/906,032

(22) Filed: Jul. 13, 2001

(51) Int. Cl. A61K 48/00

(52) U.S. Cl. 514/44

(57) ABSTRACT

The present invention provides methods for treating a defect in long term memory formation or synaptic plasticity associated with a defect in an ADF1-like molecule. The present invention also provides methods for modulating long term memory formation or synaptic plasticity by modulating Adf1-like-dependent gene expression. The present invention also provides methods for screening a pharmaceutical agent for its ability to modulate ADF1-like activity or long term memory formation.

### GOVERNMENT SUPPORT

This invention was made with Government support, in whole or in part, under Grant Numbers NIH NS32480, NIH NS34889, NIH KO2-NS02001 and NRSA NS09763, awarded by the National Institutes of Health. The United States government has certain rights in this invention.

### BACKGROUND OF THE INVENTION

Development of the nervous system is exceedingly complex and generally requires the implementation of two mechanistic strategies (Goodman and Shatz, Cell Suppl., 72:77-98 (1993)). Activity-independent mechanisms control the differentiation and identification of different neuronal classes, the elaboration and selective guidance of axonal growth cones, and the recognition of appropriate post-synaptic targets. These steps result in a stereotyped but crude pattern of synaptic connections. Activity-dependent mechanisms then promote the sharpening and refinement of these circuits through selective synapse stabilization, growth and elimination.

Well-studied vertebrate systems clearly illustrate these principles. In the visual system, for example, retinal ganglion neurons form connections with the optic tectum in lower vertebrates (Constantine-Paton et al., Annu. Rev. Neurosci., 13:129-154 (1990)) or the visual thalamus in mammals (Shatz et al., Cold Spring Harbor Symp. Quant. Biol., 55:469-480 (1990)). Activity-independent mechanisms set up a diffuse topographic map, which is sharpened by neural activity and results in coordinated innervation patterns.

A similar strategy underlies formation of the neuromuscular junction (NMJ) (Sanes and Lichtmann, Annu. Rev. Neurosci., 22:389-442 (1999)). Activity-independent guidance and recognition cues allow motor neurons to establish specific, but redundant, connections with multiple fibers within a single muscle. Activity-dependent mechanisms then sharpen this innervation pattern through selective axonal withdrawal and synapse elimination so that each fiber within the muscle receives input from only one motorneuron. These two mechanisms ensure a proper functioning circuit. The relative contribution of each mechanism, however, appears to vary, depending on the particular system and circuit (Goodman and Shatz, Cell Suppl., 72:77-98 (1993)).

Activity-dependent plasticity does not end at birth, but continues throughout the life of the animal. This allows the postnatal brain to modify and refine its activity in response to external cues. In the mammalian visual system, for example, intrinsic oscillators are supplanted by extrinsic visual inputs, which can modify synaptic connections to and within the primary visual cortex during a sensitive “critical period” (Shatz et al., Cold Spring Harbor Symp. Quant. Biol., 55:469-480 (1990)). Similarly, development of the vertebrate neuromuscular junction reflects a complex process of activity-dependent mechanisms that modulate muscle contraction (Sanes and Lichtmann, Annu. Rev. Neurosci., 22:389-442 (1999)). Perhaps even more provocatively, the findings from lesion-based studies of cortical representation areas and from electron microscopic examinations of neuronal anatomy suggest that synaptic reorganization occurs within the adult brain itself (see Kolb, Brain Plasticity and Behavior (Hillsdate, N.J.: Lawrence Erlbaum (1995)). Molecular and physiological insights also are emerging from cellular models of neuroplasticity and behavioral investigations of learning and memory. It is becoming clear that adults and developing animals can modify their synapses with a host of common mechanisms and molecular components, e.g., adhesion molecules, glutamate receptors, neurotrophins and components of cAMP signaling cascades (Cline and Constantine-Paton, Neuron, 3:413-426 (1989); Mayford et al., Science, 256:638-644 (1992); Tang et al., Nature, 401:63-69 (1999); McKay et al., Learning and Memory, 6:193-215 (1999); Pham et al., Neuron, 22:63-72 (1999); Cline, Current Biology, 8:R836-R839 (1998); Weiler et al., Behav. Brain Res., 66:1-6 (1995); Bailey and Kandel, Annu. Rev. Physiol., 55:397-426 (1993); Bear et al., J. Neurosci., 10:909-925 (1990)). These findings support the notion that mechanisms of adult learning and of developmental plasticity may be conserved.

### SUMMARY OF THE INVENTION

Applicants have discovered a novel link between molecular mechanisms of development and behavioral plasticity. In particular, Applicants have identified Adf1, a member of the myb-related family of transcription factors, as providing a novel link between molecular mechanisms of development and behavioral plasticity. As described herein, Adf1 plays a role in both development of the neuromuscular junction (NMJ) and in long term memory (LTM) formation. A defect in Adf1 results in reduced synaptic structure and memory impairment in adult animals. In particular, reduced expression of Adf1 impairs LTM formation in adult animals. An increase in expression of Adf1 results in increased synaptic structure in adult animals. Accordingly, it is expected that an increase in expression of Adf1 results in increased memory formation in adult animals. In particular, it is expected that over-expression of Adf1 enhances LTM formation in adult animals. As described herein, it is also expected that co-stimulation of the Adf1 and CREB pathways enhances neural plasticity, including LTM formation in adult animals.

As a result, the present invention provides methods for treating an animal (particularly a human, other mammal, vertebrate or invertebrate) with a defect in long term memory formation associated with a defect in an ADF1-like molecule and/or a defect in synaptic plasticity associated with a defect in an ADF1-like molecule. The animal is preferably an adult animal. The defect in ADF1-like molecule is either a diminution in the amount of ADF1-like molecule produced, a diminution in the activity or action of the ADF1-like molecule produced or both a diminution in amount and activity or action of the ADF1-like molecule. In one embodiment, the method comprises treating an animal with a defect in long term memory formation and/or synaptic plasticity associated with a defect in an ADF1-like molecule to increase expression of an Adf1-like gene relative to expression of the Adf1-like gene in the animal prior to treatment. In a second embodiment, the method comprises treating an animal with a defect in long term memory formation and/or synaptic plasticity associated with a defect in an ADF1-like molecule to increase functional ADF1-like activity relative to the functional ADF1-like activity in the animal prior to treatment. In a third embodiment, the method comprises administering to an animal with a defect in long term memory formation and/or synaptic plasticity associated with a defect in ADF1-like molecule an ADF1 compound such as an ADF1-like molecule, ADF1 analog, biologically active ADF1-like fragment or ADF1-like fusion protein. In a fourth embodiment, the method comprises administering to an animal with a defect in long term memory formation and/or synaptic plasticity associated with a defect in ADF1-like molecule a nucleic acid sequence encoding an ADF1-like molecule, ADF1 analog, biologically active ADF1-like fragment or ADF1 fusion protein. In a fifth embodiment, the method comprises treating an animal with a defect in long term memory formation and/or synaptic plasticity associated with a defect in an ADF1-like molecule by administering to the animal an effective amount of a pharmaceutical agent which is capable of increasing expression of an Adf1-like gene relative to expression of the Adf1-like gene in the animal prior to administration of the pharmaceutical agent. In a sixth embodiment, the method comprises treating an animal with a defect in long term memory formation and/or synaptic plasticity associated with a defect in an ADF1-like molecule comprising administering to the animal an effective amount of a pharmaceutical agent which is capable of increasing functional ADF1-like activity relative to the functional ADF1-like activity in the animal prior to administration of the pharmaceutical agent.

Alternatively, in one embodiment, the method for treating an animal (particularly a human, other mammal, vertebrate or invertebrate) with a defect in long term memory formation associated with a defect in an ADF1-like molecule and/or a defect in synaptic plasticity associated with a defect in an ADF1-like molecule comprises treating the animal to increase expression of an Adf1-like gene relative to expression of the Adf1-like gene in the animal prior to treatment and to enhance CREB pathway function in the animal prior to treatment. In a second embodiment, the method comprises treating an animal with a defect in long term memory formation and/or synaptic plasticity associated with a defect in an ADF1-like molecule to increase functional ADF1-like activity relative to the functional ADF1-like activity in the animal prior to treatment and to enhance CREB pathway function relative to CREB pathway function in the animal prior to treatment. In a third embodiment, the method comprises administering to an animal with a defect in long term memory formation and/or synaptic plasticity associated with a defect in ADF1-like molecule an ADF1 compound such as an ADF1-like molecule, ADF1 analog, biologically active ADF1-like fragment or ADF1-like fusion protein in conjunction with an effective amount of a compound which is capable of enhancing, CREB pathway function relative to CREB pathway function in the animal prior to administration of the compound. In a fourth embodiment, the method comprises administering to an animal with a defect in long term memory formation and/or synaptic plasticity associated with a defect in ADF1-like molecule a nucleic acid sequence encoding an ADF1-like molecule, ADF1 analog, biologically active ADF1-like fragment or ADF1 fusion protein in conjunction with an effective amount of a compound which is capable of enhancing CREB pathway function relative to CREB pathway function in the animal prior to administration of the compound. In a fifth embodiment, the method comprises treating an animal with a defect in long term memory formation and/or synaptic plasticity associated with a defect in an ADF1-like molecule by administering to the animal an effective amount of a pharmaceutical agent which is capable of increasing expression of an Adf1-like gene relative to expression of the Adf1-like gene in the animal prior to administration of the pharmaceutical agent in conjunction with an effective amount of a compound which is capable of enhancing CREB pathway function relative to CREB pathway function in the animal prior to administration of the compound. The pharmaceutical agent which is capable of increasing expression of an Adf1-like gene can be the same or different from the compound which is capable of enhancing CREB pathway function. In a sixth embodiment, the method comprises treating an animal with a defect in long term memory formation and/or synaptic plasticity associated with a defect in an ADF1-like molecule comprising administering to the animal an effective amount of a pharmaceutical agent which is capable of increasing functional ADF1-like activity relative to the functional ADF1-like activity in the animal prior to administration of the pharmaceutical agent in conjunction with an effective amount of a compound which is capable of enhancing CREB pathway function relative to CREB pathway function in the animal prior to administration of the compound. The pharmaceutical agent which is capable of increasing, functional ADF1-like activity can be the same or different from the compound which is capable of enhancing CREB pathway function.

The invention also provides methods for treating an animal (particularly a human, other mammal, vertebrate or invertebrate) with a defect in long term memory formation associated with a defect in a CREB molecule and/or a defect in synaptic plasticity associated with a defect in a CREB molecule. The animal is preferably an adult animal. The defect in CREB molecule is either a diminution in the amount of CREB molecule produced, a diminution in the activity or action of the CREB molecule produced or both a diminution in amount and activity or action of the CREB molecule. In one embodiment, the method comprises treating an animal with a defect in long term memory formation and/or synaptic plasticity associated with a defect in a CREB molecule to increase expression of an Adf1-like gene relative to expression of the Adf1-like gene in the animal prior to treatment. In a second embodiment, the method comprises treating an animal with a defect in long term memory formation and/or synaptic plasticity associated with a defect in a CREB molecule to increase functional ADF1-like activity relative to the functional ADF1-like activity in the animal prior to treatment. In a third embodiment, the method comprises administering to an animal with a defect in long term memory formation and/or synaptic plasticity associated with a defect in a CREB molecule an ADF1 compound such as an ADF1-like molecule, ADF1 analog, biologically active ADF1-like fragment or ADF1-like fusion protein. In a fourth embodiment, the method comprises administering to an animal with a defect in long term memory formation and/or synaptic plasticity associated with a defect in a CREB molecule a nucleic acid sequence encoding an ADF1-like molecule, ADF1 analog, biologically active ADF1-like fragment or ADF1 fusion protein. In a fifth embodiment, the method comprises treating an animal with a defect in long term memory formation and/or synaptic plasticity associated with a defect in a CREB molecule by administering to the animal an effective amount of a pharmaceutical agent which is capable of increasing expression of an Adf1-like gene relative to expression of the Adf1-like gene in the animal prior to administration of the pharmaceutical agent. In a sixth embodiment, the method comprises treating an animal with a defect in long term memory formation and/or synaptic plasticity associated with a defect in a CREB molecule comprising administering to the animal an effective amount of a pharmaceutical agent which is capable of increasing functional ADF1-like activity relative to the functional ADF1-like activity in the animal prior to administration of the pharmaceutical agent.

Alternatively, in one embodiment, the method for treating an animal (particularly a human, other mammal, vertebrate or invertebrate) with a defect in long term memory formation associated with a defect in a CREB molecule and/or a defect in synaptic plasticity associated with a defect in a CREB molecule comprises treating the animal to increase expression of an Adf1-like gene relative to expression of the Adf1-like gene in the animal prior to treatment and to enhance CREB pathway function in the animal prior to treatment. In a second embodiment, the method comprises treating an animal with a defect in long term memory formation and/or synaptic plasticity associated with a defect in a CREB molecule to increase functional ADF1-like activity relative to the functional ADF1-like activity in the animal prior to treatment and to enhance CREB pathway function relative to CREB pathway function in the animal prior to treatment. In a third embodiment, the method comprises administering to an animal with a defect in long term memory formation and/or synaptic plasticity associated with a defect in a CREB molecule an ADF1 compound such as an ADF1-like molecule, ADF1 analog, biologically active ADF1-like fragment or ADF1-like fusion protein in conjunction with an effective amount of a compound which is capable of enhancing, CREB pathway function relative to CREB pathway function in the animal prior to administration of the compound. In a fourth embodiment, the method comprises administering to an animal with a defect in long term memory formation and/or synaptic plasticity associated with a defect in a CREB molecule a nucleic acid sequence encoding an ADF1-like molecule, ADF1 analog, biologically active ADF1-like fragment or ADF1 fusion protein in conjunction with an effective amount of a compound which is capable of enhancing CREB pathway function relative to CREB pathway function in the animal prior to administration of the compound. In a fifth embodiment, the method comprises treating an animal with a defect in long term memory formation and/or synaptic plasticity associated with a defect in a CREB molecule by administering to the animal an effective amount of a pharmaceutical agent which is capable of increasing expression of an Adf1-like gene relative to expression of the Adf1-like gene in the animal prior to administration of the pharmaceutical agent in conjunction with an effective amount of a compound which is capable of enhancing CREB pathway function relative to CREB pathway function in the animal prior to administration of the compound. The pharmaceutical agent which is capable of increasing expression of an Adf1-like gene can be the same or different from the compound which is capable of enhancing CREB pathway function. In a sixth embodiment, the method comprises treating an animal with a defect in long term memory formation and/or synaptic plasticity associated with a defect in a CREB molecule comprising administering to the animal an effective amount of a pharmaceutical agent which is capable of increasing functional ADF1-like activity relative to the functional ADF1-like activity in the animal prior to administration of the pharmaceutical agent in conjunction with an effective amount of a compound which is capable of enhancing CREB pathway function relative to CREB pathway function in the animal prior to administration of the compound. The pharmaceutical agent which is capable of increasing, functional ADF1-like activity can be the same or different from the compound which is capable of enhancing CREB pathway function.

The invention further relates to methods of modulating long term memory formation and/or synaptic plasticity in an animal, preferably an adult animal, comprising treating the animal to modulate Adf1-like-dependent gene expression. In one embodiment, the method comprises treating the animal to modulate expression of an Adf1-like gene. In a particular embodiment, the method comprises administering to the animal an effective amount of a pharmaceutical agent which is capable of modulating expression of an Adf1-like gene in the animal. Alternatively, the method comprises treating the animal to modulate functional ADF1-like activity. In a particular embodiment, the method comprises administering to the animal an effective amount of a pharmaceutical agent which is capable of modulating functional ADF1-like activity in the animal.

The invention relates to methods of modulating long term memory formation and/or synaptic plasticity in an animal, preferably in an adult animal, comprising treating the animal to modulate Adf1-like-dependent gene expression and CREB-dependent gene expression. In one embodiment, the method comprises treating the animal to modulate expression of an Adf1-like gene and a CREB gene. In a particular embodiment, the method comprises administering to the animal an effective amount of a pharmaceutical agent which is capable of modulating expression of an Adf1-like gene in the animal in conjunction with an effective amount of a pharmaceutical agent which is capable of modulating expression of a CREB gene in the animal. The pharmaceutical agent which is capable of modulating expression of an Adf1-like gene can be the same or different from the pharmaceutical agent which is capable of modulating expression of a CREB gene. Alternatively, the method comprises treating the animal to modulate functional ADF1-like activity and functional (biologically active) CREB. In a particular embodiment, the method comprises administering to the animal an effective amount of a pharmaceutical agent which is capable of modulating functional ADF1-like activity in the animal in conjunction with an effective amount of a pharmaceutical agent which is capable of modulating functional CREB in the animal. The pharmaceutical agent which is capable of modulating functional ADF1-like activity can be the same or different from the pharmaceutical agent which is capable of modulating functional CREB.

The present invention further relates to methods of enhancing long term memory formation and/or synaptic plasticity in an animal, preferably an adult animal, comprising treating the animal to modulate Adf1-like-dependent gene expression. In one embodiment, the method comprises treating the animal to increase expression of an Adf1-like gene relative to expression of the Adf1-like gene in the animal prior to treatment. In a second embodiment, the method comprises treating the animal to increase functional ADF1-like activity relative to the functional ADF1-like activity in the animal prior to treatment. In a third embodiment, the method comprises administering to the animal an effective amount of an ADF1-like molecule, ADF1 analog, biologically active ADF1-like fragment or ADF1-like fusion protein. In a fourth embodiment, the method comprises administering to the animal an effective amount of a nucleic acid sequence encoding an ADF1-like molecule, ADF1 analog, biologically active ADF1-like fragment or ADF1-like fusion protein. In a fifth embodiment, the method comprises administering to the animal an effective amount of a pharmaceutical agent which is capable of increasing expression of an Adf1-like gene relative to expression of the Adf1-like gene in the animal prior to administration of the pharmaceutical agent. In a sixth embodiment, the method comprises administering to the animal an effective amount of a pharmaceutical agent which is capable of increasing functional ADF1-like activity relative to the functional ADF1-like activity in the animal prior to administration of the pharmaceutical agent.

The invention relates to methods of enhancing long term memory formation and/or synaptic plasticity in an animal, preferably an adult animal, comprising treating the animal to modulate Adf1-like-dependent gene expression and CREB-dependent gene expression. In one embodiment, the method comprises treating the animal to increase expression of an Adf1-like gene relative to expression of the Adf1-like gene in the animal prior to treatment and to enhance CREB pathway function in the animal prior to treatment. In a second embodiment, the method comprises treating the animal to increase functional ADF1-like activity relative to the functional ADF1-like activity in the animal prior to treatment and to enhance CREB pathway function relative to CREB pathway function in the animal prior to treatment. In a third embodiment, the method comprises administering to the animal an effective amount of an ADF1-like molecule, ADF1 analog, biologically active ADF1-like fragment or ADF1-like fusion protein in conjunction with an effective amount of a compound which is capable of enhancing CREB pathway function relative to CREB pathway function in the animal prior to administration of the compound. In a fourth embodiment, the method comprises administering to the animal an effective amount of a nucleic acid sequence encoding an ADF1-like molecule, ADF1 analog, biologically active ADF1-like fragment or ADF1-like fusion protein in conjunction with an effective amount of a compound which is capable of enhancing CREB pathway function relative to CREB pathway function in the animal prior to administration of the compound. In a fifth embodiment, the method comprises administering to the animal an effective amount of a pharmaceutical agent which is capable of increasing expression of an Adf1-like gene relative to expression of the Adf1-like gene in the animal prior to administration of the pharmaceutical agent in conjunction with an effective amount of a compound which is capable of enhancing CREB pathway function relative to CREB pathway function in the animal prior to administration of the compound. The pharmaceutical agent which is capable of increasing expression of an Adf1-like gene can be the same or different from the compound which is capable of enhancing CREB pathway function. In a sixth embodiment, the method comprises administering to the animal an effective amount of a pharmaceutical agent which is capable of increasing functional ADF1-like activity relative to the functional ADF1-like activity in the animal prior to administration of the pharmaceutical agent in conjunction with an effective amount of a compound which is capable of enhancing CREB pathway function relative to CREB pathway function in the animal prior to administration of the compound. The pharmaceutical agent which is capable of increasing functional ADF1-like activity can be the same or different from the compound which is capable of enhancing CREB pathway function.

The invention also relates to methods of screening a pharmaceutical agent for its ability to modulate ADF1-like activity in an animal, preferably an adult animal (particularly an animal (e.g., human, other mammal, vertebrate or invertebrate) with a defect in long term memory formation associated with a defect in an ADF1-like molecule) comprising (a) administering the pharmaceutical agent to the animal; and (b) determining the functional ADF1-like activity in the animal obtained in step (a) relative to the functional ADF1-like activity in a control animal to which the pharmaceutical has not been administered. A difference in the functional ADF1-like activity determined in the animal treated with the pharmaceutical agent relative to the functional ADF1-like activity determined in the control animal identifies the pharmaceutical agent as one having the ability to modulate ADF1-like activity in the animal.

The invention further relates to methods of screening a pharmaceutical agent for its ability to modulate long term memory formation in an animal, preferably an adult animal (particularly an animal (e.g., human, other mammal, vertebrate or invertebrate) with a defect in long term memory formation associated with a defect in an ADF1-like molecule) comprising (a) administering the pharmaceutical agent to the animal; (b) determining the functional ADF1-like activity in the animal obtained in step (a) relative to the functional ADF1-like activity in a control animal to which the pharmaceutical has not been administered; (c) selecting the animal in step (b) having a functional ADF1-like activity which differs from the functional ADF1-like activity in the control animal to which the pharmaceutical agent has not been administered; (d) training the animal selected in step (c) under conditions appropriate to produce long term memory formation in the animal; (e) assessing long term memory formation in the animal trained in step (d); and (f) comparing long term memory formation assessed in step (e) with long term memory formation produced in the control animal to which the pharmaceutical agent has not been administered. A difference in long term memory formation assessed in the animal treated with the pharmaceutical agent relative to the long term memory formation assessed in the control animal identifies the pharmaceutical agent as one which has the ability to modulate long term memory formation in the animal.

In another embodiment of screening a pharmaceutical agent for its ability to modulate long term memory formation, the method comprises (a) administering a pharmaceutical agent to an animal having an inducible Adf1-like gene; (b) inducing expression of the Adf1-like gene in the animal; (c) training the animal under conditions appropriate to produce long term memory formation in the animal; (d) assessing long term memory formation in the animal trained in step (c); and (e) comparing long term memory formation assessed in step (d) with long term memory formation produced in a control animal to which the pharmaceutical agent has not been administered. If a difference is noted in long term memory formation assessed in the animal treated with the pharmaceutical agent relative to the long term memory formation assessed in the control animal, the pharmaceutical agent can be categorized as having the ability to modulate long term memory formation in the animal.

The invention also relates to methods of assessing the effect of a pharmaceutical agent on ADF1-like activity in an animal, preferably an adult animal (particularly an animal (e.g., human, other mammal, vertebrate or invertebrate) with a defect in long term memory formation associated with a defect in an ADF1-like molecule) comprising (a) administering the pharmaceutical agent to the animal; and (b) determining the functional ADF1-like activity in the animal obtained in step (a) relative to the functional ADF1-like activity in a control animal to which the pharmaceutical has not been administered. A difference in the functional ADF1-like activity determined in the animal treated with the pharmaceutical agent relative to the functional ADF1-like activity determined in the control animal identifies the pharmaceutical agent as one having an effect on ADF1-like activity in the animal.

The invention further relates to methods for assessing the effect a pharmaceutical agent on long term memory formation in an animal, preferably an adult animal (particularly an animal (e.g., human, other mammal, vertebrate or invertebrate) with a defect in long term memory formation associated with a defect in an ADF1-like molecule) comprising (a) administering the pharmaceutical agent to the animal; (b) determining the functional ADF1-like activity in the animal obtained in step (a) relative to the functional ADF1-like activity in a control animal to which the pharmaceutical has not been administered; (c) selecting the animal in step (b) having a functional ADF1-like activity which differs from the functional ADF1-like activity in the control animal to which the pharmaceutical agent has not been administered; (d) training the animal selected in step (c) under conditions appropriate to produce long term memory formation in the animal; (e) assessing long term memory formation in the animal trained in step (d); and (f) comparing long term memory formation assessed in step (e) with long term memory formation produced in the control animal to which the pharmaceutical agent has not been administered. A difference in long term memory formation assessed in the animal treated with the pharmaceutical agent relative to the long term memory formation assessed in the control animal identifies the pharmaceutical agent as one having an effect on long term memory formation in the animal.

In another embodiment for assessing the effect a pharmaceutical agent on long term memory formation, the method comprises (a) administering a pharmaceutical agent to an animal having an inducible Adf1-like gene; (b) inducing expression of the Adf1-like gene in the animal; (c) training the animal under conditions appropriate to produce long term memory formation in the animal; (d) assessing long term memory formation in the animal trained in step (c); and (e) comparing long term memory formation assessed in step (d) with long term memory formation produced in a control animal to which the pharmaceutical agent has not been administered. If a difference is noted in long term memory formation assessed in the animal treated with the pharmaceutical agent relative to the long term memory formation assessed in the control animal, the pharmaceutical agent can be categorized as having an effect on long term memory formation in the animal.

The invention further relates to methods of screening for or identifying a pharmaceutical agent which is capable of modulating ADF1-like activity comprising (a) introducing into host cells particularly cells of neural origin (e.g., neuroblastomas, neurons, neural stem cells)) a DNA construct, wherein the DNA construct comprises (1) DNA encoding an indicator gene and (2) a promoter sequence which comprises an ADF1 binding site and is operably linked to the DNA encoding the indicator gene; (b) producing a sample by introducing into host cells comprising the DNA construct a pharmaceutical agent to be assessed for its ability to modulate ADF1-like activity under conditions appropriate for expression of the indicator gene; (c) detecting expression of the indicator gene in the sample obtained in step (b); and (d) comparing expression detected in step (c) with expression of the indicator gene detected in control cells into which the pharmaceutical agent has not been introduced. A difference in expression of indicator gene in the sample obtained in step b) compared to the expression of indicator gene in control cells identifies the pharmaceutical agent as one which modulates ADF1-like activity.

The invention also relates to the novel antibodies (immunoglobulins) described herein specific for ADF1 and antigen-binding fragments thereof.

### BRIEF DESCRIPTION OF THE DRAWINGS

FIGS. 1A-1D depict results showing that the nal^{P1} mutation produces a specific disruption of Pavlovian olfactory learning.

FIG. 1A is a graphic representation of performance of wildtype and nal^{P1} mutant flies (2-3 days old), assayed at 0, 15, 30, 60, 180 and 360 minutes after a single training session of the classical conditioning procedure of Tully and Quinn (J. Comp. Physiol., 157:263-277 (1985)). Performance of nal^{P1} mutants is significantly lower than normal at all time points. Memory decay rates did not differ between mutant and wildtype strains, indicating normal memory formation processes in both and suggesting a learning deficit in nal^{P1} mutants. n=6 PIs per group.

FIG. 1B is a schematic diagram of fifty independent excisions of the nal^{P1} P element insertion that were generated via the crossing scheme of Dura et al. (J. Neurogenet., 9:1-14 (1993)). Some (nal^{ve25A}, nal^{ve80}, and nal^{ve96}) are homozygous viable and molecularly precise. Others (nal^{le55} and nal^{le60}) are homozygous lethal and molecularly imprecise, with deletions of nearby genomic DNA. The nal^{le60} excision consists of 30 bp of residual P element sequences, an 8 bp (target site) duplication and an approximately 1,800 bp deletion of genomic DNA. The nal^{ve48} excision retains the same P element/duplication sequences as nal^{le60} but does not carry the genomic deletion.

FIG. 1C is a bar graph representation of the effect on olfactory memory in homozygous and heteroallelic combinations of precise excisions (nal^{ve25A}, nal^{ve80} and nal^{ve96}). Olfactory memory was assayed immediately after one training session. In each case, performance levels did not differ significantly from those of two wildtype strains [w¹¹¹⁸(CS10) and Canton-S], and all were significantly higher than that of nal^{P1} mutants. These data indicate that the precise excisions are revertant for olfactory learning and, thereby, establish that the original nal^{P1} transposon insertion is responsible for the mutant memory defect. n=6 PIs per group.

FIG. 1D is a bar graph representation of the effect on olfactory memory in homozygous, heterozygous and heteroallelic combinations of nal^{P1} and imprecise excisions (nal^{ve48} and nal^{le60}). Olfactory memory was assayed immediately after one training session. Both excision alleles are recessive to the wildtype allele for olfactory memory (and lethality). The nal^{le60} mutation fails to complement nal^{P1}. In heteroallelic combinations, olfactory memory was significantly reduced in nal^{P1}/nal^{le60} flies but did not differ significantly from wildtype in nal^{P1}/nal^{ve48} flies. Taken together, these genetic complementation data indicate that the olfactory memory defect maps to the molecular deletion, but not the P element/duplication sequences, of the nal^{le60} excision mutation. n=14, 14, 14, 8, 8, 14, 8, 8, and 14 PIs per group, respectively.

FIG. 2A show intron/exon molecular mapping of Adf1 and cn20. The cn20 transcription unit produces a novel, unspliced transcript. The nal^{P1} P element (arrow) is inserted within the major intron of the Adf1 transcription unit. The Adf1 transcription unit is alternatively spliced into (at least) two mRNAs, which may employ two different translation start sites, i1 and i2. The extent of the genomic deletion in nal^{le60} is indicated. Restriction sites: B, Bam HI; E, EcoRI; H, Hind III; S, Sac II.

FIGS. 2B-2C depict results showing that the nal^{P1} P element is inserted in the Adf1 gene, reducing levels of Adf1 RNA and protein.

FIG. 2B is a photograph depicting results of Northern blot analyses of Adf1 and cn20 in mutant nal^{P1} and wildtype flies. Poly(A) RNA was isolated from adult heads (lanes 1 and 2) and bodies (lanes 3 and 4) of each genotype. Relative to control levels of rp49 RNA, cn20 mRNA expression levels in both heads and bodies are similar in wildtype and mutant flies. In contrast, Adf1 mRNA expression levels are reduced by at least two-fold in mutant heads and bodies.

FIG. 2C is a photograph depicting results of Western blot analysis of ADF1 protein levels in nal^{P1} and wildtype adult heads. Three different concentrations of protein were loaded for each genotype (from lowest to highest in lanes 1-3 (50 μg, 75 μg, 100 μg) and highest to lowest in lanes 4-6 (100 μg, 75 μg, 50 μg)). Relative to control levels of two other proteins (TATA-binding protein (TBP) and α-tubulin (TUBULIN), ADF1 expression is reduced at least two-fold in mutant flies.

FIGS. 3A-3E depict results showing that developmentally leaky, but not adult-acute, expression of an hsp-adf1⁺ transgene rescues the early memory defect of nal^{P1} mutants or disrupts early memory when over-expressed in wildtype flies.

FIG. 3A is a photograph depicting results of Northern blot analyses of Adf1 RNA in wildtype, mutant and transgenic adults grown at 18° C. (odd numbered lanes) or 25° C. (even numbered lanes). The hsp-adf1⁺ transgene (hsp-Adf1⁺-8, -11 and -32) shows leaky levels of expression at 18° C., with a further increase at 25° C.

FIG. 3B is a bar graph representation of performance levels in wildtype, nal^{P1}, hsp-Adf1⁺-8, hsp-Adf1⁺-11, hsp-Adf1⁺-32 and nal^{P1};hsp-Adf1⁺-11 flies. When grown at 25° C. performance levels immediately after one training session for each transgenic line are lower than normal (but still significantly higher than that of nal^{P1} mutants). Leaky expression of hsp-Adf1⁺-11 in nal^{P1} mutant flies still yields defective olfactory memory. n=6 PIs per group.

FIG. 3C is a bar graph representation of performance levels in wildtype, nal^{P1}, hsp-Adf1⁺-8, hsp-Adf1⁺-11, hsp-Adf1⁺-32 and nal^{P1};hsp-Adf1⁺-11 flies. Adults were grown at 18° C. and equilibrated to 25° C. just prior to training by a shift to RT for 45 minutes, then to 25° C. for 45 minutes. Performance levels were higher in the transgenic lines. n=8 PIs per group.

FIG. 3D is a bar graph representation of performance levels in wildtype, nal^{P1}, hsp-Adf1⁺-8, hsp-Adf1⁺-11, hsp-Adf1⁺-32 and nal^{P1};hsp-Adf1⁺-11 flies. When grown at 18° C. and then shifted to 25° C. for three days after eclosion, leaky expression of hsp-Adf1⁺-11 appears sufficient to rescue the early memory deficit of nal^{P1};hsp-Adf1⁺-11 transgenic flies. n=6 PIs per group.

FIG. 3E is a bar graph representation of performance levels in wildtype, hsp-Adf1⁺-8;nal^{P1} and nal^{P1};hsp-Adf1⁺-11 flies. Flies were grown at 25° C. and then subjected to one 30 minute heat shock at 37° C., which was sufficient to induce detectable levels of ADF1 (Western blot below each group) three hours before one training session. The deficient performance levels of hsp-Adf1⁺-8;nal^{P1} and nal^{P1};hsp-Adf1⁺-11, normally produced without heat shock (−HS), remained deficient after heat shock (+HS). n=6 PIs per group.

FIGS. 4A-4D depict results showing that ADF1 protein expression is widespread in the developing and adult nervous system and reduced in nal mutants.

FIG. 4A is a photograph depicting results of Western blot analysis of adult wildtype head extracts. A monoclonal antibody MAb Adf1-17 (1:100) (lane 1) or the antecedent mouse polyclonal sera (1:10,000) (lane 2) conferred highly specific recognition of a single protein species of ˜34 kDa.

FIG. 4B is a photograph of embryo images showing in situ expression of ADF1 protein in wildtype and mutant stage 16 embryos. MAb Adf1-17 was used to compare the pattern and intensity of ADF1 expression at stage 16. ADF1 shows widespread and strictly nuclear expression in wildtype embryos. ADF1 levels are uniformly reduced in heterozygous embryos (+/nal^{le60}), further reduced in hypomorphic nal^{P1} embryos and undetectable in null embryos (nal^{le60}/nal^{le60}).

FIG. 4C is a pair of photographs of larval central nervous system (CNS) images showing in situ expression of ADF1 in larval CNS. MAb Adf1-17 was visualized with an immunofluorescent second antibody in wildtype and mutant third instar whole-mount brains. Widespread expression is seen in wildtype (+) tissue (and cell nuclei at higher magnification) and is uniformly reduced in hypomorphic nal^{P1} mutants.

FIG. 4D is a pair of photographs of adult head images showing in situ expression of ADF1 in wildtype and mutant adult heads. MAb Adf1-17 immunoreactivity in 10 micron frontal sections from frozen heads again reveals widespread nuclear expression in wildtype flies (+) and a uniform reduction in hypomorphic nal^{P1} mutants.

FIGS. 5A-5D depict results showing that Adf1 modulates synapse formation at the larval neuromuscular junction (NMJ).

FIG. 5A is a set of photographs of synaptic boutons labeled with an antibody against synaptotagmin and viewed on a laser scanning confocal microscope. Compared to wildtype (+), nal^{P1} hypomorphs have fewer synaptic boutons, while hsp-Adf1⁺-8 transgenic larvae grown at 21° C. have more. The distribution of synaptotagmin within individual boutons is normal, and each muscle is innervated by large (type 1b) and small (type 1s) boutons, indicating that they are dually innervated.

FIG. 5B is a bar graph representation of the numbers of synaptic boutons quantified in wildtype flies (+), in hypomorphic nal^{P1} mutants, in transgenic flies over-expressing hsp-Adf1⁺ on a wildtype background (hsp-Adf1⁺-8; hsp-Adf1⁺-11) and in transgenic flies expressing hsp-Adf1⁺ on a mutant background (nal^{P1};hsp-Adf1⁺-11). Over- or under-expression of Adf1 produced fewer or more synaptic boutons as above. In contrast, expression of the transgene on a mutant background yielded normal numbers of synaptic boutons.

FIG. 5C is a pair of traces of evoked synaptic transmission (upper traces) and of mEJC amplitudes and frequencies (lower panel) in wildtype, mutant and transgenic flies. Synaptic currents were recorded from muscle 6 in segment A2 using two-electrode voltage clamp. Each data point represents the average of 2-3 EJCs per preparation, and there was no evidence of significant variation between EJCs recorded in different trains in the same preparation. All genotypes showed similar evoked currents. Representative traces of mEJC amplitudes and frequencies (lower panel) were determined from recording spontaneous activity in muscle 6 in each preparation above.

FIG. 5D is a set of bar graph representations of plots of EJC and mEJC amplitude, mEJC frequency, and quantal content. Each EJC sample represents the average of 2-3 EJCs per preparation. mEJC amplitude and frequency were calculated from all mEJCs in a 40.875 seconds record. All physiological parameters are normal in nal^{P1} mutants and hsp-Adf1⁺-11 transgenic larvae, while hsp-Adf1⁺-8 animals show a non-specific increase in mEJC frequency.

FIGS. 6A and 6B depict results showing that long-term memory is preferentially disrupted in nal^{P1} mutants.

FIG. 6A is a bar graph representation of results showing one-day memory retention after massed and spaced training in wildtype flies (+) and nal^{P1} mutants. Spaced training normally produces a memory retention level twice that of massed training in wild-type flies. Such memory retention after massed training is a direct measure of the ARM memory component, while that after spaced training is composed of roughly equal amounts of ARM and LTM (Tully et al., Cell, 79:35-47 (1994)). Massed training produced normal memory in nal^{P1} mutants (P=0.994), suggesting that ARM is normal. In contrast, spaced training yielded significantly lower memory retention in nal^{P1} mutants than in wildtype flies. In fact, performance levels after spaced and massed training in the mutants were similar to those after massed training in wild-type flies. n=16 PIs per group.

FIG. 6B is a bar graph representation showing 7-day memory after spaced training in wildtype flies (+) and nal^{P1} mutants. In wildtype flies, memory retention at this interval is greater than zero only if protein synthesis- and CREB-dependent LTM is induced by spaced training (Tully et al., Cell, 79:35-47 (1994); Yin et al., Cell, 79:49-58 (1994)). Thus, 7-day memory is a direct measure of LTM. Memory retention of nal^{P1} mutants is significantly lower than that in wildtype flies (P=0.001) and is not significantly different from zero (P=0.363), indicating loss of LTM. n=6 PIs per group.

FIG. 7 is a schematic diagram depicting the genetic pathway of developmental plasticity at the neuromuscular junction (NMJ). Synapse growth at the Drosophila NMJ involves at least two genetic pathways leading to: (1) an increase in synaptic sprouting (structure) and (2) and an increase in synaptic transmission (function). The sum effect of these two processes is enhanced synaptic strength. These pathways share both common and distinct genetic components, as proposed by Davis et al. (Neuron, 17:669-679 (1996)). Both are stimulated by increased neuronal excitability, such as that caused by ether-a-go-go;shaker double mutants or by increased cAMP levels, such as that caused by dunce mutants. In turn, cAMP signaling is proposed to enhance synaptic strength by expanding synaptic structure, which can be induced by Fas II hypomorphs, and incorporating into the expanded structure CREB-dependent release machinery. The studies described herein extend this model by showing that the structural pathway may be modulated through Adf1-mediated transcription, not just through local modulation of molecules at the synapse, such as Fas II. In addition, the results described herein show that Adf1, like dCREB2, plays a specific modulatory role: genetic mutations that decrease and increase Adf1 expression have opposing effects on synaptic structure but show no disruptions of basal synaptic function. Thus, Adf1 and dCREB2 define distinct transcription factor cascades involved in synaptic plasticity.

### DETAILED DESCRIPTION OF THE INVENTION

Activity-dependent plasticity is well described in Drosophila. Detailed studies of Pavlovian olfactory learning have revealed behavioral properties similar to other invertebrates and to vertebrates, suggesting genetic conservation of the underlying machinery (DeZazzo and Tully, Trends Neurosci., 18:212-217 (1995)). Molecular studies have confirmed this view by establishing a role for cAMP signaling (see Dubnau and Tully, Annu. Rev. Neurosci., 21:407-444 (1998)). This cascade extends from the cell surface to the nucleus and operates in at least two temporal domains: a short-term phase involving the covalent modification of proteins and a long-term phase requiring the synthesis of RNA and protein. At the transition between these two phases appears to be a conserved molecular switch controlled by the activity of the cAMP-responsive transcription factor CREB.

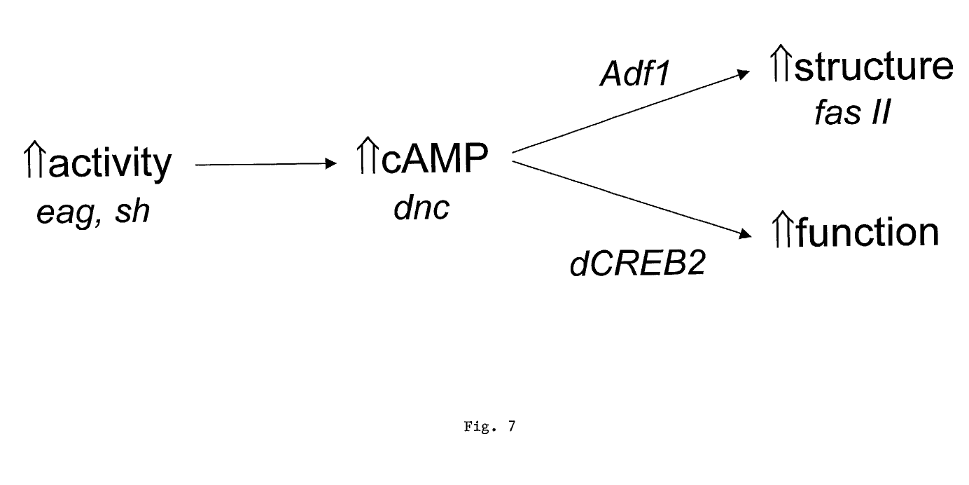

In concert with behavioral studies, synaptic plasticity in Drosophila also has been analyzed at the developing neuromuscular junction (NMJ) (Gramates and Budnik, In International Review of Neurobiology, Budnik and Gramates, eds. (New York: Academic Press) pp. 93-117 (1999); Hannan and Zhong, In International Review of Neurobiology, Budnik and Gramates, eds. (New York: Academic Press) pp. 119-128 (1999)). The Drosophila NMJ is similar to vertebrate excitatory synapses: it is glutamatergic, organized into boutons and displays activity-dependent plasticity. Mutants that disrupt adult olfactory memory, mostly via cAMP signaling, can also disrupt NMJ structure and function. These studies have culminated in a model (Davis et al., Neuron, 17:669-679 (1996); FIG. 7), in which neuronal activity increases cAMP and then enhances synaptic strength via the activation of structural and functional effectors.

As described herein, Adf1, like dCREB2, has been shown to play a role in both development of the NMJ and in LTM formation. At the NMJ, the role of Adf1 appears distinct from, and complementary to, that of dCREB2. dCREB2 affects synaptic function but not structure, while Adf1 affects synaptic structure but not function. Collectively, the findings described herein suggest that Adf1 and dCREB2 are critical regulatory switches of distinct transcriptional cascades involved in terminal stages of synapse maturation.

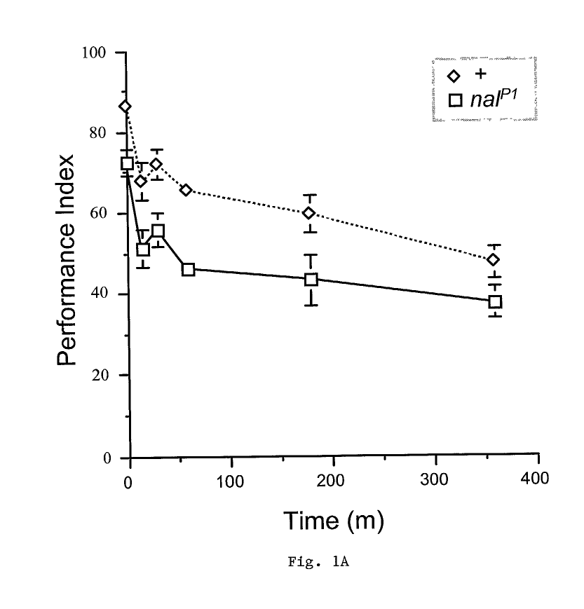

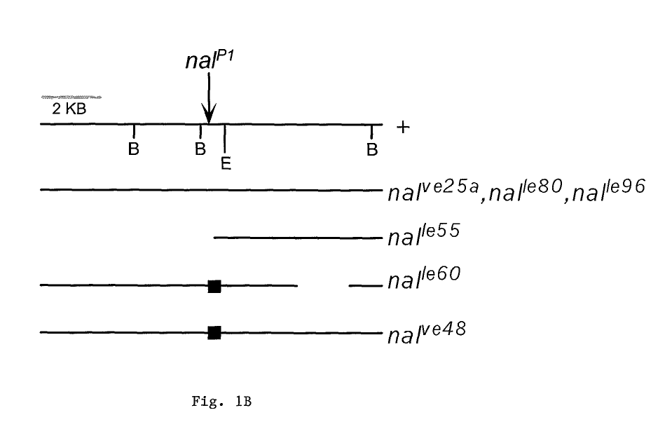

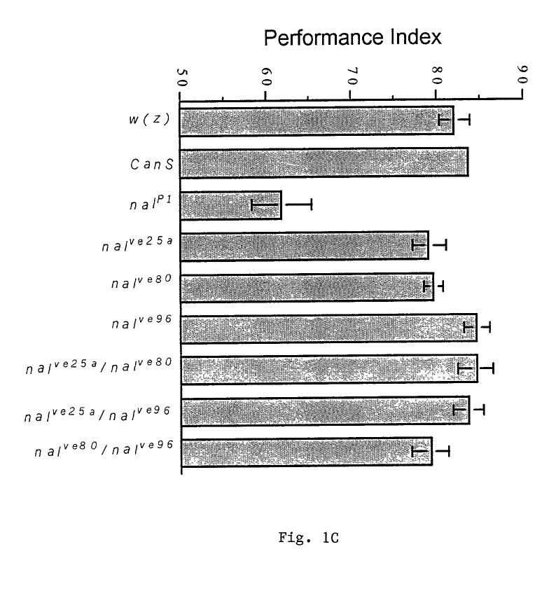

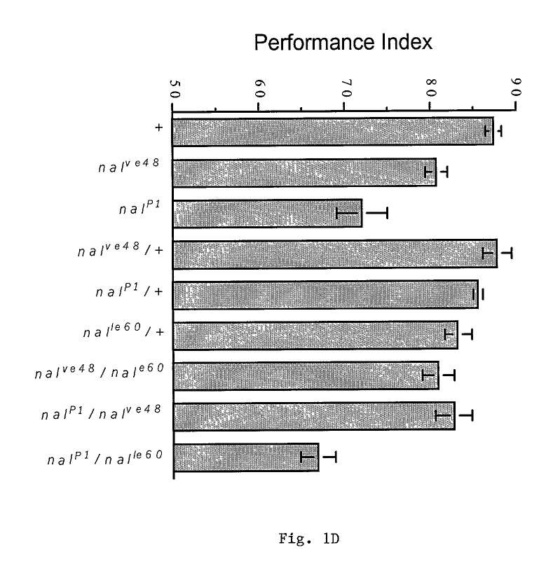

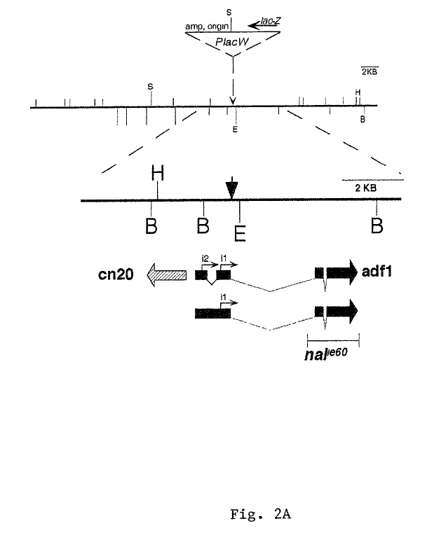

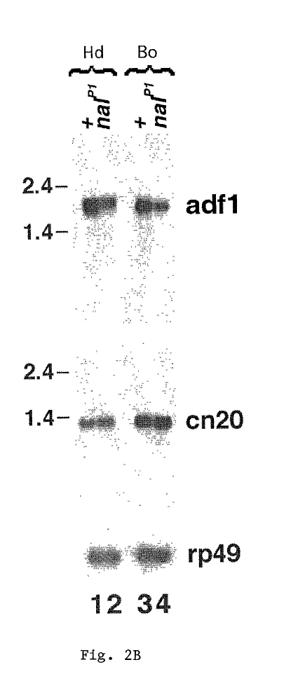

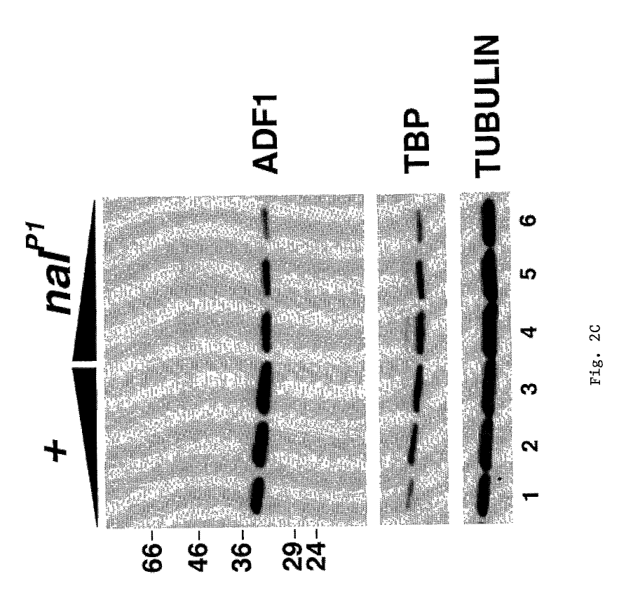

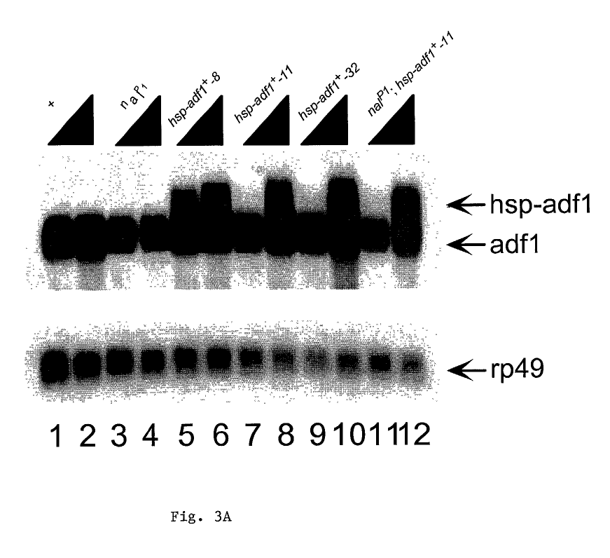

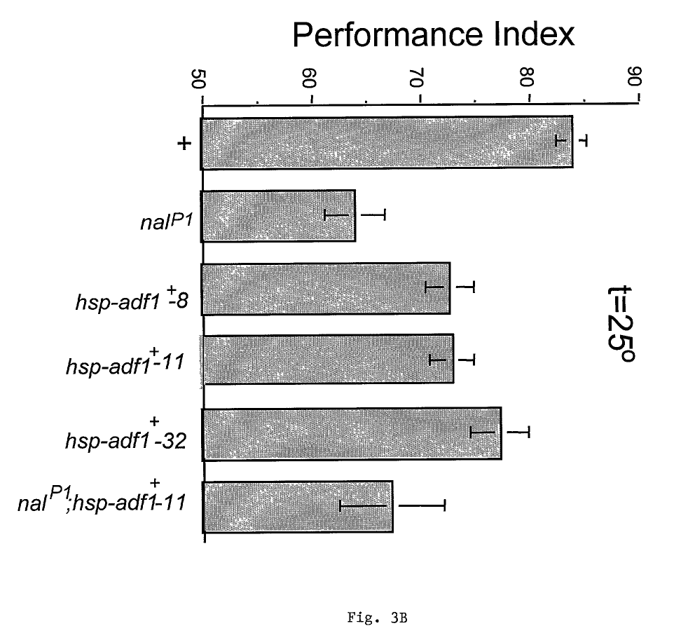

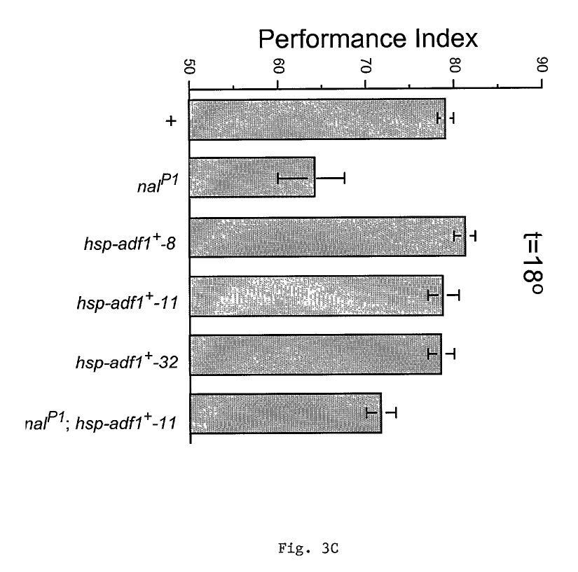

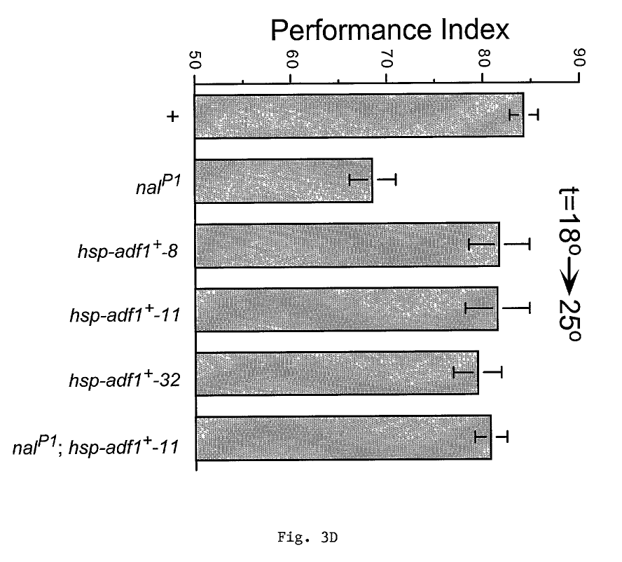

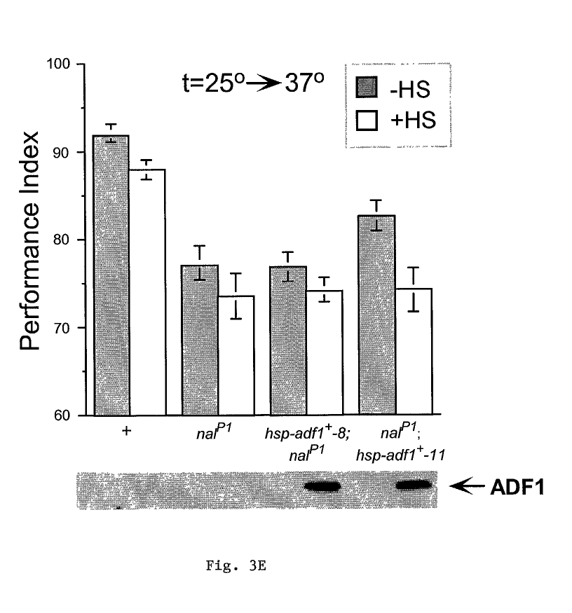

A novel olfactory memory mutant nalyot (nal) is described herein. nal encodes the Adf1 transcription factor, a myb-related protein, which likely impinges upon many downstream targets. Multiple lines of convergent evidence conclusively link the nal^{P1} olfactory memory defect to the Adf1 gene. First, the nal P element is inserted in a major intron of the Adf1 transcription unit, and its precise excision reverts the memory defect (FIGS. 1A-1D). Second, the nal^{P1} memory defect is not complemented by the (homozygous-lethal) imprecise excision allele nal^{le60}, which carries a small deletion that removes only Adf1 coding sequences (FIGS. 1A-1D and 2A-2C). Third, the nal^{P1} mutation reduces levels of expression of Adf1 RNA and protein, but does not affect RNA levels of the neighboring transcription unit cn20 (FIGS. 2A-2C). Fourth, three independent transgenic lines, each driving leaky over-expression of Adf1, show an adult memory deficit (FIGS. 3A-3E). Finally, the nal^{P1} memory defect is rescued with an hsp-Adf1⁺ transgene expressing the wild-type Adf1 protein (FIGS. 3A-3E).

The molecular etiology of nal^{P1} is similar to that of other P transposant mutants in general, and to other memory mutants in particular. The memory mutants Volado (Grotewiel et al., Nature, 391:455-460 (1998)), leonardo (Skoulakis et al., Neuron, 17:931-944 (1996)) and NF1 (Guo et al., Nature, 403:895-898 (2000) each arise from a P insertion into an intron; latheo (Boynton et al., Genetics, 131:655-672 (1992)), DCO (Skoulakis et al., Neuron, 11:197-208 (1993)) and amnesiac (DeZazzo et al., J. Neurosci., 19:8740-8746 (1999)) each arise from insertion into an exon; and the linotte mutation (Bolwig et al., Neuron, 15:829-842 (1995)) derives from a P element insertion in the 3′ genomic region.

Transgenic over-expression experiments described herein indicate that optimal memory formation requires tight developmental control of Adf1 expression. Perturbing this regulation temporally or spatially can disrupt memory formation and viability. These findings reinforce regulatory themes emerging from behavioral studies of other transcription factors. Transgenic mice expressing two additional copies of the clock transcription factor, for instance, show defective circadian function (Antoch et al., Cell, 89:655-667 (1997)). More to the point, acute heat shock of a stonewall transgene, which also encodes a myb-related transcription factor, leads to death (Clark and McKearin, Development, 122:937-950 (1996)).

Phenotypic rescue through basal, or “leaky” transgene expression, is also a well established phenomenom. In fact, basal transgene expression completely or partially rescues the Volado memory deficit (Grotewiel et al., Nature, 391:455-460 (1998)) and period circadian rhythm deficits (Ewer et al., Nature, 333:82-84 (1988)) and transforms foraging food searching behavior (Osborne et al., Science, 277:834-836 (1997)). For stonewall, fertility defects of the mutants are rescued by leaky expression of an hsp-stw⁺ transgene (Clark and McKearin, Development, 122:937-950 (1996)).

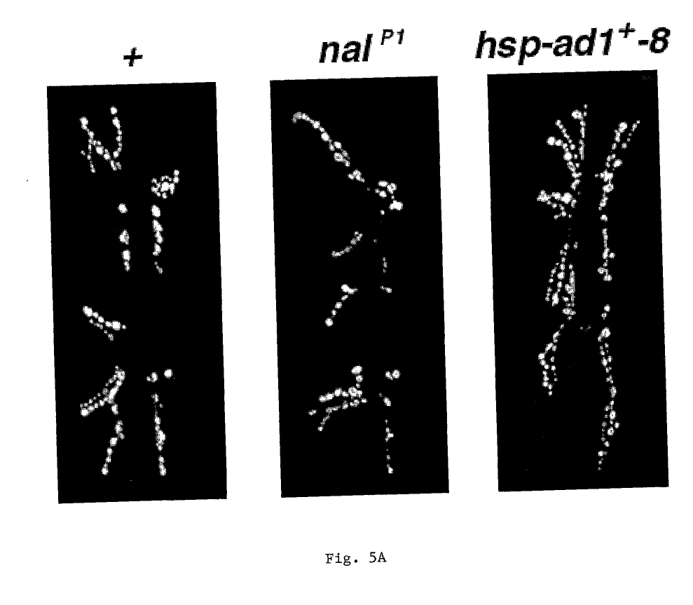

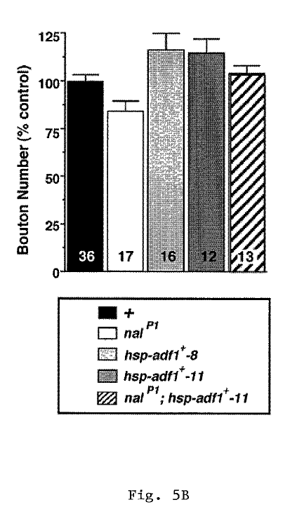

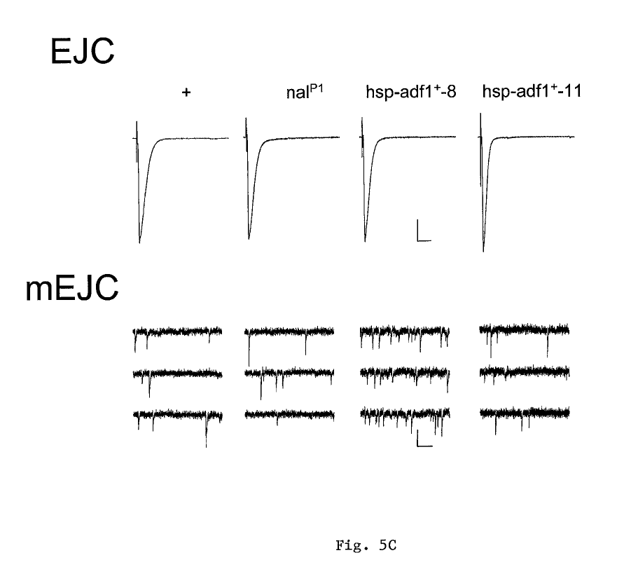

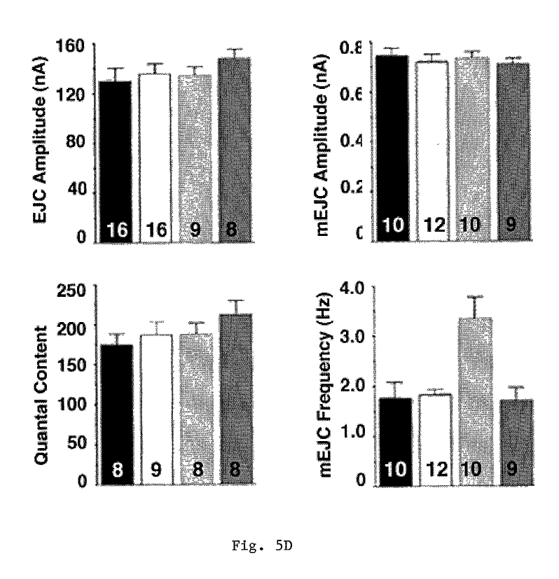

Adf1 is required both during development and in the adult for proper memory formation. Several lines of investigation suggest a complex, multi-functional role for Adf1 in the nervous system. First, Adf1 shows widespread expression throughout development. However, null mutants survive to the late embryonic or early larval stage, and they fail to reveal any discernible disruptions of gross morphology in the nervous system. Similarly, adult-viable (hypomorphic) Adf1 mutants show normal neuropilar volume of adult mushroom bodies and the central complex, neuroanatomical centers implicated in adult olfactory memory. Nevertheless, over-expression of Adf1⁺ kills the animals, even when transgenic expression is restricted to the nervous system. Adf1 therefore may play an essential role in terminal stages of neuronal differentiation and function. Second, leaky over-expression of hsp-Adf1⁺ during development leads to adult memory defects. Early memory is reduced in all three +; hsp-Adf1⁺ lines when they are raised at 25° C., but not at 18° C. (FIGS. 3B and 3C). Concomitantly, leaky hsp-Adf1⁺ expression is much greater at 25° C. than at 18° C. (FIG. 3A). Thus, normal adult memory formation may require tight developmental regulation of Adf1. Third, chronic expression of Adf1⁺ in adults is sufficient to rescue the nal memory defect. Rescue in nal^{P1}; hsp-Adf1⁺-11 mutant transgenics was complete when these animals were raised during development at 18° C. and then shifted as adults to 25° C. for three days (FIG. 3D). Finally, genetic mutations that increase or decrease levels of ADF1 protein at the NMJ have reciprocal effects on bouton number (FIGS. 5A-5E). Once again, leaky transgene expression is capable of rescuing the defect in bouton number observed in nal^{P1} mutants.

The complex etiology of nal may not be unusual. The Vol locus, for example, yields at least two transcripts, Vol-l and Vol-s, each encoding the same protein (Grotewiel et al., Nature, 391:455-460 (1998)). Acute induction of Vol-s (which is expressed in both head and body tissues) was sufficient to rescue fully the early memory defect of a Vol-s null mutant. The Vol-s mutant nevertheless shows normal levels of the head-specific Vol-l transcript. A developmental role for Vol in adult behavior cannot be excluded, however, since Vol-l may subserve this role. Thus, inducible transgene experiments to date have addressed whether acute expression of a transgene in adults is necessary for rescue of a memory defect, but they have not yet resolved whether such expression is sufficient.

Adf1 modulates maturation of synaptic structure at the NMJ. Formation of functional synapses at the larval NMJ reflects an ongoing activity-dependent process that begins during late embryogenesis (Broadie and Bate, Neuron, 11:607-619 (1993); Saitoe et al., Dev. Biol., 184:48-60 (1997)) and continues throughout larval development (Gramates and Budnik, In International Review of Neurobiology, Budnik and Gramates, eds. (New York: Academic Press), pp. 93-117 (1999); Hannan and Zhong, In International Review of Neurobiology, Burnik and Gramates, eds. (New York: Academic Press), pp. 119-128 (1999)). Initial events in synapse formation, however, do not require neuronal activity. In contrast, the subsequent maturation of synaptic branches and boutons during larval development clearly is activity-dependent and can be modulated by changes in neuronal excitability, in cAMP signaling, or in expression of cell adhesion molecules (Hannan and Zhong, In International Review of Neurobiology, Burnik and Gramates, eds. (New York: Academic Press), pp. 119-128 (1999)).

Developmental plasticity at the NMJ has been genetically dissected (Davis et al., Neuron, 17:669-679 (1996); FIG. 7). Neuronal activity triggers a cAMP signaling cascade, leading to an increase in both synaptic bouton number (structure) and the quantal content of synaptic transmission (function). The structural and functional pathways appear to be independent of each other, involving the cell adhesion molecule fasciclin II (Fas II) and CREB transcription factor, respectively. Genetic mutations that decrease Fas II expression to about 50% wild-type levels increase bouton number without causing a corresponding change in synaptic strength. Similarly, over-expression of a CREB repressor (dCREB2-b) in a dunce mutant background (which constitutively elevates cAMP levels and increases the number of synaptic boutons) blocks the mutant increase in synaptic strength but not the mutant increase in synaptic structure. Conversely, overexpression of a CREB activator (dCREB2-a) has no effect at a wild-type (normal) NMJ but increases synaptic function at a mutant Fas II NMJ (after this mutation first yields an increase in synaptic structure).

Several genes have now been implicated in growth of the Drosophila neuromuscular synapse. Fas II is expressed pre- and post-synaptically at the NMJ, where it appears to act as a cell adhesion molecule in a manner analogous to its vertebrate NCAM homolog. More recent discoveries, made with the use of genetic screens, include the identification of Highwire (Wan et al., Neuron, 26:313-329 (2000)), a novel gene with expression localized to periactive zones of presynaptic terminals, and Futsch (Hummel et al., Neuron, 26:357-370 (2000); Roos et al., Neuron, 26:371-382 (2000)), encoding a MAP1B-like protein associated with the axonal, dendritic and nerve-terminal cytoskeleton. Evidence is provided herein for a transcription factor, Adf1, involved in the structural pathway. Genetic manipulations that decrease (or increase) the amount of ADF1 give rise to a decrease (or increase) in synaptic bouton number. Thus, whereas dCREB2 affects NMJ function but not structure, Adf1 affects structure but not function (FIG. 7). As a transcription factor, ADF1 appears not to function directly at the synapse. Rather, its role reasonably may act upstream of structural effectors (as above). Together, these observations suggest that transcriptional regulation is involved with both components of synapse maturation, and they argue that an increase in synaptic structure must precede an increase in synaptic function.

Multiple signaling pathways may lie upstream of Adf1. Genetic manipulations of cAMP signaling affect NMJ structure and function (Davis et al., Neuron, 20:305-315 (1998)). Similarly, Adf1 activity also may be regulated by covalent modification. An examination of the Adf1 protein sequence reveals several serine residues that represent potential sites of convergence by a number of kinases, including PKA and CamKII. One of these residues, Ser-164, maps to the transactivation region and appears to be specifically phorphorylated in vitro by CamKII. Phosphorylation of this residue, or its mutation to an alanine or acidic residue, reduces transcriptional activation. Notably, these modification only partly reduce activity, consistent with the findings described herein that Adf1 is under subtle regulatory control. CAMKII is expressed in the NMJ and functions acutely to modulate synaptic structure and function (Wang et al., Neuron., 13:1373-1384 (1994); Griffith et al., Proc. Natl. Acad. Sci. USA, 91:10044-10048 (1994); Koh et al., Cell, 98:353-363 (1999)). Over-expression of an inhibitor peptide against CAMKII leads to an increase in the number of synaptic boutons—an observation consistent with the ADF1 biochemistry described herein and with results from ADF1 over-expression studies. Thus, ADF1 may be downstream of one or both of these signaling pathways in the NMJ. More generally, pharmacological and genetic disruptions of CAMKII or PKA signaling also produce deficits in mammalian long-tern potentiation and learning (Wong et al., Neuron, 23:787-798 (1999); Mayford et al., Science, 274:1678-1683 (1996); Makhinson et al., J. Neurosci., 19:2500-2510 (1999)), suggesting that Adf1-mediated transcription may play a phylogenetically conserved role in synaptic plasticity.

Adf1 and Early Memory.

Developmental plasticity in the adult brain may share some of the cellular machinery that subserves synapse maturation at the NMJ. During metamorphosis, for instance, axonal projections from some larval mushroom body neurons first degenerate and then extend processes anew along with new MB neurons that proliferate in developing adult structures (Barth and Heisenberg, Learning and Memory, 4:219-229 (1997)). This process of synapse formation continues for a few days after eclosion, is modulated in an experience-dependent fashion (Technau, J. Neurogenet., 1:113-126 (1984); Heisenberg et al., J. Neurosci., 15:1851-1960 (1995); Barth and Heisenberg, Learning and Memory, 4:219-229 (1997)), and is aberrant in mutants with defects in cAMP signaling (Balling et al., J. Neurogenet., 4:65-73 (1987)) or in normal larvae grown in low density cultures (Heisenberg et al., J. Neurosci., 15:1851-1960 (1995)).

Positing a role for ADF1 in activity-dependent synapse formation of the maturing adult brain can explain why hypomorphic mutations produce mild defects in olfactory memory measured immediately after one training session. Several observations are consistent with this notion. First, leaky over-expression of hs-Adf1 during development disrupts memory. Second, over-expressing the ADF1 protein in the nervous system via the UAS-GAL4 system has lethal consequences. Third, Adf1 mutants have a defect in synaptic structure at the neuromuscular junction. These observations suggest that subtle structural defects at central synapses at least partly underlie the nal memory defect.

Nevertheless, the nal memory defect is not accompanied by any overt morphological changes in the embryonic or adult nervous system. In fact, normal adult morphology of the mushroom bodies and central complex were observed even in severe allelic combinations (nal^{P1}/nal^{le60}) predicted to lower ADF1 protein levels to less than 25% of wild-type type. These observations suggest that defects in synaptic structure occur at very terminal stages of synaptic maturation after the growth of axons and dendrites, which represent the main contributions to planimetric measurements of neuropil (de Belle and Heisenberg, Proc. Natl. Acad. Sci. USA, 93:9875-9880 (1996); Heisenberg et al., J. Neurosci., 15:1851-1960 (1995)). In this context, the behavioral analyses described herein reveal that assays of adult olfactory memory provide a sensitive way to detect subtle developmental abnormalities that escape morphological detection.

Adf1 and Long-Term Memory.

Existence of a protein synthesis-dependent LTM appears ubiquitous in the animal kingdom and has been shown to be CREB-dependent in mammals (Bourtchuladze et al., Cell, 79:59-68 (1997); Gass et al., Learning and Memory, 5:274-288 (1998); Kogan et al., Current Biology, 7:1-11 (1998); Oike et al., Human Mol. Genet., 8:387-396 (1990); Guzowski and McGaugh, Proc. Natl. Acad. Sci. USA, 94:2693-2698 (1997); Lamprecht et al., J. Neurosci., 17:8443-8450 (1997); Josselyn et al., Society for Neuroscience, 24:926, Abstract 365.10 (1998)). Long-term memory formation after Pavlovian training in Drosophila also depends on CREB-dependent gene transcription and protein synthesis (Yin et al., Cell., 79:49-58 (1994); Tully et al., Cell., 79:35-47 (1994)). Moreover, opposite manipulations of CREB have corresponding loss- and gain-of-function effects on long-term memory formation (Yin et al., Cell, 79:49-58 (1994); Yin et al., Cell, 81:107-115 (1995)), implying that CREB acts as a molecular switch for LTM formation—as it does for activity dependent plasticity at the NMJ (Davis et al., Neuron, 17:669-679 (1996)). Manipulations of CREB in cultured molluscan neurons have demonstrated activity-induced structural changes at identified sensorimotor synapses concomitant with the appearance of long-term facilitation, a cellular correlate of behavioral sensitization (Dash et al., Proc. Natl. Acad. Sci. USA, 88:5061-5065 (1991); Bartsch et al., Cell, 83:979-992 (1995); Bartsch et al., Cell, 95:211-213 (1998)). Hence, the appearance of long-term memory generally may include structural, as well as functional, changes at the relevant synapses.

Long-term memory formation after spaced training is abolished in nal mutants. A trivial explanation for this result is that it derives secondarily from the milder deficit in early memory. Two observations argue against this interpretation. First, one-day memory after massed training is normal in nal mutants, indicating that ARM is formed normally and, thereby, suggesting that multiple training sessions compensate for the mild memory deficit observed after one training session. Second, radish mutants have a more severe early memory deficit than nal mutants (and ARM is abolished) but nevertheless show normal LTM formation (Tully et al., Cell, 79:35-47 (1994)). Thus, the level of performance at earlier memory phases is not a reliable predictor of performance at later memory stages. These observations also do not readily support a general developmental etiology of the nal LTM deficit, unless early memory and LTM are anatomically distinct.

Instead, as described herein, observations at the NMJ suggest a possible structural role for Adf1 in adult behavioral plasticity. During the formation of LTM, the Adf1 transcriptional cascade may lead to an increase in the number of synaptic boutons, allowing the incorporation of synaptic machinery induced through the CREB transcription cascade. In nal mutants, structural changes do not occur, thereby preventing integration of the CREB-dependent increases in synaptic function, as is the case at the NMJ in the absence of FasII- or dunce-dependent increases in synaptic boutons. Without an increase in structure and function, there is no increase in synaptic strength, and LTM does not manifest.

As a transcription factor, Adf1 may also regulate the expression of functional components of neuronal activity, which are compensated for in the homeostatic context of the NMJ, but which are not compensated for in the adult CNS. Experiments with spatially and temporally restricted transgenes can be employed to distinguish these possible roles for Adf1 in LTM formation.

Transcription Factor Cascades for Neuroplasticity.

Adf1 is a complex gene. It is essential during early development and also contributes to developmental and behavioral plasticity. While these functions may seem to be disparate, a closer examination suggests a common mechanistic thread. First, the essential requirement for Adf1 during development does not appear to be at the level of proliferation, but instead appears to reflect a more specialized post-mitotic role. In Adf1 null mutants, gross morphology is normal, the majority of embryos develop to maturity and a significant fraction hatch. These observations suggest that Adf1 may guide the terminal stages of cellular differentiation and maintenance, as is the case for other Myb family members (Oh and Reddy, Oncogene, 18:3017-3033 (1999); Clark and McKearin, Development, 122:937-950 (1996); Martin and Paz-Ares, Trends Genet., 13:67-73 (1997); Wang and Tobin, Cell, 93:1207-1217 (1998)). In neurons, such terminal stages likely include synapse formation and refinement. In larvae, this process occurs chronically in the activity-dependent growth of neuromuscular synapses. In adults, this process may occur acutely, and in a much more spatially restricted pattern, during the formation of specific long-term memories.

These various spatiotemporal roles for Adf1 support the growing notion that developmental plasticity and adult behavioral plasticity reflect similar cellular processes. The genetic approach described herein strengthens this biological insight in two novel ways. First, the original nal mutation is identified in a “forward-genetic” screen for defective adult plasticity. In this manner, no assumptions were made about any a priori connections between development and adult function. Second, “reverse-genetic” manipulations of Adf1 yield similar effects on synapse formation at the NMJ and on adult memory, thereby suggesting a mechanistic link between these temporally (and spatially) disparate processes.

Given ADF1's role as a transcription factor, an obvious question is: What are the downstream targets of Adf1 involved in synapse formation? While a number of candidate targets, including Alcohol dehydrogenase (Adh) and Dopa decarboxylase (Ddc), have been identified in vitro (England et al., J. Biol. Chem., 265:5086-5094 (1990)), the significance of these observations in vivo is unclear. For example, Northern analysis reveals no decrease of Adh RNA levels in nal heads. Similarly, the putative Adf1 binding site of Ddc drives expression in the embryonic hypoderm where the molecule is involved in hardening of cuticle, yet no obvious cuticular defects are seen in Adf1 null mutants. More to the point, synaptic proteins under Adf1 regulation have not yet been identified. In this regard, Fas II, Highwire, and Futsch are obvious candidates, given their established role in synaptic growth at the NMJ. ADF1 effectors can be identified by determining whether a particular effector affects the ability of Adf1 to modulate synaptic morphology directly during LTM formation.

The present invention encompasses methods of treating an animal with a defect in long term memory formation associated with a defect in an ADF1-like molecule and/or a defect in synaptic plasticity associated with a defect in an ADF1-like molecule. In one embodiment, the method comprises treating an animal with a defect in long term memory formation and/or synaptic plasticity associated with a defect in an ADF1-like molecule to increase expression of an Adf1-like gene relative to expression of the Adf1-like gene in the animal prior to treatment. In a second embodiment, the method comprises treating an animal with a defect in long term memory formation and/or synaptic plasticity associated with a defect in an ADF1-like molecule to increase functional ADF1-like activity relative to the functional ADF1-like activity in the animal prior to treatment. In a third embodiment, the method comprises administering to an animal with a defect in long term memory formation and/or synaptic plasticity associated with a defect in ADF1-like molecule an ADF1 compound such as an ADF1-like molecule, ADF1 analog, biologically active ADF1-like fragment or ADF1-like fusion protein. In a fourth embodiment, the method comprises administering to an animal with a defect in long term memory formation and/or synaptic plasticity associated with a defect in ADF1-like molecule a nucleic acid sequence encoding an ADF1-like molecule, ADF1 analog, biologically active ADF1-like fragment or ADF1 fusion protein. In a fifth embodiment, the method comprises treating an animal with a defect in long term memory formation and/or synaptic plasticity associated with a defect in an ADF1-like molecule by administering to the animal an effective amount of a pharmaceutical agent which increases, or is capable of increasing, expression of an Adf1-like gene relative to expression of the Adf1-like gene in the animal prior to administration of the pharmaceutical agent. In a sixth embodiment, the method comprises treating an animal with a defect in long term memory formation and/or synaptic plasticity associated with a defect in an ADF1-like molecule comprising administering to the animal an effective amount of a pharmaceutical agent which increases, or is capable of increasing, functional ADF1-like activity relative to the functional ADF1-like activity in the animal prior to administration of the pharmaceutical agent.

Alternatively, in one embodiment, the method of treating an animal with a defect in long term memory formation associated with a defect in an ADF1-like molecule and/or a defect in synaptic plasticity associated with a defect in an ADF1-like molecule comprises treating the animal to increase expression of an Adf1-like gene relative to expression of the Adf1-like gene in the animal prior to treatment and to enhance CREB pathway function in the animal prior to treatment. In a second embodiment, the method comprises treating an animal with a defect in long term memory formation and/or synaptic plasticity associated with a defect in an ADF1-like molecule to increase functional ADF1-like activity relative to the functional ADF1-like activity in the animal prior to treatment and to enhance CREB pathway function relative to CREB pathway function in the animal prior to treatment. In a third embodiment, the method comprises administering to an animal with a defect in long term memory formation and/or synaptic plasticity associated with a defect in ADF1-like molecule an ADF1 compound such as an ADF1-like molecule, ADF1 analog, biologically active ADF1-like fragment or ADF1-like fusion protein in conjunction with an effective amount of a compound which enhances, or is capable of enhancing, CREB pathway function relative to CREB pathway function in the animal prior to administration of the compound. In a fourth embodiment, the method comprises administering to an animal with a defect in long term memory formation and/or synaptic plasticity associated with a defect in ADF1-like molecule a nucleic acid sequence encoding an ADF1-like molecule, ADF1 analog, biologically active ADF1-like fragment or ADF1 fusion protein in conjunction with an effective amount of a compound which enhances, or is capable of enhancing, CREB pathway function relative to CREB pathway function in the animal prior to administration of the compound. In a fifth embodiment, the method comprises treating an animal with a defect in long term memory formation and/or synaptic plasticity associated with a defect in an ADF1-like molecule by administering to the animal an effective amount of a pharmaceutical agent which increases, or is capable of increasing, expression of an Adf1-like gene relative to expression of the Adf1-like gene in the animal prior to administration of the pharmaceutical agent in conjunction with an effective amount of a compound which enhances, or is capable of enhancing, CREB pathway function relative to CREB pathway function in the animal prior to administration of the compound. The pharmaceutical agent which increases, or is capable of increasing, expression of an Adf1-like gene can be the same or different from the compound which enhances, or is capable of enhancing, CREB pathway function. In a sixth embodiment, the method comprises treating an animal with a defect in long term memory formation and/or synaptic plasticity associated with a defect in an ADF1-like molecule comprising administering to the animal an effective amount of a pharmaceutical agent which increases, or is capable of increasing, functional ADF1-like activity relative to the functional ADF1-like activity in the animal prior to administration of the pharmaceutical agent in conjunction with an effective amount of a compound which enhances, or is capable of enhancing, CREB pathway function relative to CREB pathway function in the animal prior to administration of the compound. The pharmaceutical agent which increases, or is capable of increasing, functional ADF1-like activity can be the same or different from the compound which enhances, or is capable of enhancing, CREB pathway function.

As used herein, a defect in long term memory formation associated with a defect in an ADF1-like molecule or a defect in synaptic plasticity associated with a defect in an ADF1-like molecule can be a recently acquired defect or a long term defect (e.g., a developmentally acquired defect). The defect in ADF1-like molecule is either a diminution in the amount of ADF1-like molecule produced, a diminution in the activity or action of the ADF1-like molecule produced or both a diminution in amount and activity or action of the ADF1-like molecule. By “defect in ADF1-like molecule” is meant a defect in ADF1-like pathway function (Adf1-like-dependent gene expression).

The invention also encompasses methods of treating an animal with a defect in long term memory formation associated with a defect in a CREB molecule and/or a defect in synaptic plasticity associated with a defect in a CREB molecule. In one embodiment, the method comprises treating an animal with a defect in long term memory formation and/or synaptic plasticity associated with a defect in a CREB molecule to increase expression of an Adf1-like gene relative to expression of the Adf1-like gene in the animal prior to treatment. In a second embodiment, the method comprises treating an animal with a defect in long term memory formation and/or synaptic plasticity associated with a defect in a CREB molecule to increase functional ADF1-like activity relative to the functional ADF1-like activity in the animal prior to treatment. In a third embodiment, the method comprises administering to an animal with a defect in long term memory formation and/or synaptic plasticity associated with a defect in a CREB molecule an ADF1 compound such as an ADF1-like molecule, ADF1 analog, biologically active ADF1-like fragment or ADF1-like fusion protein. In a fourth embodiment, the method comprises administering to an animal with a defect in long term memory formation and/or synaptic plasticity associated with a defect in a CREB molecule a nucleic acid sequence encoding an ADF1-like molecule, ADF1 analog, biologically active ADF1-like fragment or ADF1 fusion protein. In a fifth embodiment, the method comprises treating an animal with a defect in long term memory formation and/or synaptic plasticity associated with a defect in a CREB molecule by administering to the animal an effective amount of a pharmaceutical agent which is capable of increasing expression of an Adf1-like gene relative to expression of the Adf1-like gene in the animal prior to administration of the pharmaceutical agent. In a sixth embodiment, the method comprises treating an animal with a defect in long term memory formation and/or synaptic plasticity associated with a defect in a CREB molecule comprising administering to the animal an effective amount of a pharmaceutical agent which is capable of increasing functional ADF1-like activity relative to the functional ADF1-like activity in the animal prior to administration of the pharmaceutical agent.

Alternatively, in one embodiment, the method of treating an animal with a defect in long term memory formation associated with a defect in a CREB molecule and/or a defect in synaptic plasticity associated with a defect in a CREB molecule comprises treating the animal to increase expression of an Adf1-like gene relative to expression of the Adf1-like gene in the animal prior to treatment and to enhance CREB pathway function in the animal prior to treatment. In a second embodiment, the method comprises treating an animal with a defect in long term memory formation and/or synaptic plasticity associated with a defect in a CREB molecule to increase functional ADF1-like activity relative to the functional ADF1-like activity in the animal prior to treatment and to enhance CREB pathway function relative to CREB pathway function in the animal prior to treatment. In a third embodiment, the method comprises administering to an animal with a defect in long term memory formation and/or synaptic plasticity associated with a defect in a CREB molecule an ADF1 compound such as an ADF1-like molecule, ADF1 analog, biologically active ADF1-like fragment or ADF1-like fusion protein in conjunction with an effective amount of a compound which is capable of enhancing, CREB pathway function relative to CREB pathway function in the animal prior to administration of the compound. In a fourth embodiment, the method comprises administering to an animal with a defect in long term memory formation and/or synaptic plasticity associated with a defect in a CREB molecule a nucleic acid sequence encoding an ADF1-like molecule, ADF1 analog, biologically an active ADF1-like fragment or ADF1 fusion protein in conjunction with an effective amount of a compound which is capable of enhancing CREB pathway function relative to CREB pathway function in the animal prior to administration of the compound. In a fifth embodiment, the method comprises treating an animal with a defect in long term memory formation and/or synaptic plasticity associated with a defect in a CREB molecule by administering to the animal an effective amount of a pharmaceutical agent which is capable of increasing expression of an Adf1-like gene relative to expression of the Adf1-like gene in the animal prior to administration of the pharmaceutical agent in conjunction with an effective amount of a compound which is capable of enhancing CREB pathway function relative to CREB pathway function in the animal prior to administration of the compound. The pharmaceutical agent which is capable of increasing expression of an Adf1-like gene can be the same or different from the compound which is capable of enhancing CREB pathway function. In a sixth embodiment, the method comprises treating an animal with a defect in long term memory formation and/or synaptic plasticity associated with a defect in a CREB molecule comprising administering to the animal an effective amount of a pharmaceutical agent which is capable of increasing functional ADF1-like activity relative to the functional ADF1-like activity in the animal prior to administration of the pharmaceutical agent in conjunction with an effective amount of a compound which is capable of enhancing CREB pathway function relative to CREB pathway function in the animal prior to administration of the compound. The pharmaceutical agent which is capable of increasing, functional ADF1-like activity can be the same or different from the compound which is capable of enhancing CREB pathway function.

As used herein, a defect in long term memory formation associated with a defect in a CREB molecule or a defect in synaptic plasticity associated with a defect in a CREB molecule can be a recently acquired defect or a long term defect (e.g., a developmentally acquired defect). The defect in CREB molecule is either a diminution in the amount of CREB molecule produced, a diminution in the activity or action of the CREB molecule produced or both a diminution in amount and activity or action of the CREB molecule. By “defect in CREB molecule” is meant a defect in CREB pathway function (CREB-dependent gene expression).

The present invention also encompasses methods of modulating long term memory formation and/or synaptic plasticity in an animal, comprising treating the animal to modulate Adf1-like-dependent gene expression. In one embodiment, the method comprises treating the animal to modulate expression of an Adf1-like gene. In a particular embodiment, the method comprises administering to the animal an effective amount of a pharmaceutical agent which modulates, or is capable of modulating, expression of an Adf1-like gene in the animal. Alternatively, the method comprises treating the animal to modulate functional ADF1-like activity. In a particular embodiment, the method comprises administering to the animal an effective amount of a pharmaceutical agent which modulates, or is capable of modulating, functional ADF1-like activity in the animal.

The invention encompasses methods of modulating long term memory formation and/or synaptic plasticity in an animal, comprising treating the animal to modulate Adf1-like-dependent gene expression and CREB-dependent gene expression. In one embodiment, the method comprises treating the animal to modulate expression of an Adf1-like gene and a CREB gene. In a particular embodiment, the method comprises administering to the animal an effective amount of a pharmaceutical agent which modulates, or is capable of modulating, expression of an Adf1-like gene in the animal in conjunction with an effective amount of a pharmaceutical agent which modulates, or is capable of modulating, expression of a CREB gene in the animal. The pharmaceutical agent which modulates, or is capable of modulating, expression of an Adf1-like gene can be the same or different from the pharmaceutical agent which modulates, or is capable of modulating, expression of a CREB gene. Alternatively, the method comprises treating the animal to modulate functional ADF1-like activity and functional (biologically active) CREB. In a particular embodiment, the method comprises administering to the animal an effective amount of a pharmaceutical agent which modulates, or is capable of modulating, functional ADF1-like activity in the animal in conjunction with an effective amount of a pharmaceutical agent which modulates, or is capable of modulating, functional CREB in the animal. The pharmaceutical agent which modulates, or is capable of modulating, functional ADF1-like activity can be the same or different from the pharmaceutical agent which modulates, or is capable of modulating, functional CREB.

The present invention further encompasses methods of enhancing long term memory formation and/or synaptic plasticity in an animal, comprising treating the animal to modulate Adf1-like-dependent gene expression. In one embodiment, the method comprises treating the animal to increase expression of an Adf1-like gene relative to expression of the Adf1-like gene in the animal prior to treatment. In a second embodiment, the method comprises treating the animal to increase functional ADF1-like activity relative to the functional ADF1-like activity in the animal prior to treatment. In a third embodiment, the method comprises administering to the animal an effective amount of an ADF1-like molecule, ADF1 analog, biologically active ADF1-like fragment or ADF1-like fusion protein. In a fourth embodiment, the method comprises administering to the animal an effective amount of a nucleic acid sequence encoding an ADF1-like molecule, ADF1 analog, biologically active ADF1-like fragment or ADF1-like fusion protein. In a fifth embodiment, the method comprises administering to the animal an effective amount of a pharmaceutical agent which increases, or is capable of increasing, expression of an Adf1-like gene relative to expression of the Adf1-like gene in the animal prior to administration of the pharmaceutical agent. In a sixth embodiment, the method comprises administering to the animal an effective amount of a pharmaceutical agent which increases, or is capable of increasing, functional ADF1-like activity relative to the functional ADF1-like activity in the animal prior to administration of the pharmaceutical agent.

The invention encompasses methods of enhancing long term memory formation and/or synaptic plasticity in an animal, comprising treating the animal to modulate Adf1-like-dependent gene expression and CREB-dependent gene expression. In one embodiment, the method comprises treating the animal to increase expression of an Adf1-like gene relative to expression of the Adf1-like gene in the animal prior to treatment and to enhance CREB pathway function in the animal prior to treatment. In a second embodiment, the method comprises treating the animal to increase functional ADF1-like activity relative to the functional ADF1-like activity in the animal prior to treatment and to enhance CREB pathway function relative to CREB pathway function in the animal prior to treatment. In a third embodiment, the method comprises administering to the animal an effective amount of an ADF1-like molecule, ADF1 analog, biologically active ADF1-like fragment or ADF1-like fusion protein in conjunction with an effective amount of a compound which enhances, or is capable of enhancing, CREB pathway function relative to CREB pathway function in the animal prior to administration of the compound. In a fourth embodiment, the method comprises administering to the animal an effective amount of a nucleic acid sequence encoding an ADF1-like molecule, ADF1 analog, biologically active ADF1-like fragment or ADF1-like fusion protein in conjunction with an effective amount of a compound which enhances, or is capable of enhancing, CREB pathway function relative to CREB pathway function in the animal prior to administration of the compound. In a fifth embodiment, the method comprises administering to the animal an effective amount of a pharmaceutical agent which increases, or is capable of increasing, expression of an Adf1-like gene relative to expression of the Adf1-like gene in the animal prior to administration of the pharmaceutical agent in conjunction with an effective amount of a compound which enhances, or is capable of enhancing, CREB pathway function relative to CREB pathway function in the animal prior to administration of the compound. The pharmaceutical agent which increases, or is capable of increasing, expression of an Adf1-like gene can be the same or different from the compound which enhances, or is capable of enhancing, CREB pathway function. In a sixth embodiment, the method comprises administering to the animal an effective amount of a pharmaceutical agent which increases, or is capable of increasing, functional ADF1-like activity relative to the functional ADF1-like activity in the animal prior to administration of the pharmaceutical agent in conjunction with an effective amount of a compound which enhances, or is capable of enhancing, CREB pathway function relative to CREB pathway function in the animal prior to administration of the compound. The pharmaceutical agent which increases, or is capable of increasing, functional ADF1-like activity can be the same or different from the compound which enhances, or is capable of enhancing, CREB pathway function.

As used herein, the term “animal” includes mammals, as well as other animals, vertebrate and invertebrate (e.g., birds, fish, reptiles, insects (e.g., Drosophila species), mollusks (e.g., Aplysia). The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals). Examples of mammalian species include humans and primates (e.g., monkeys, chimpanzees), rodents (e.g., rats, mice, guinea pigs) and ruminents (e.g., cows, pigs, horses). The animal is preferably an adult animal.

“Modulating”, as the term is used herein, includes induction, enhancement, potentiation, reduction, blocking, inhibition (total or partial) and regulation. By “regulation”, as the term is used herein, is meant the ability to control the rate and extent to which a process occurs.

By “enhancing” or “enhancement” is meant the ability to potentiate, increase, improve or make greater or better, relative to normal, a biochemical or physiological action or effect. For example, enhancing long term memory formation refers to the ability to potentiate or increase long term memory formation in an animal relative to the normal long term memory formation of the animal. As a result, long term memory acquisition is faster or better retained. Enhancing synaptic plasticity refers to the ability to potentiate or improve synaptic plasticity in an animal relative to the normal synaptic plasticity of the animal. The term “synaptic plasticity” is an art-recognized term and is used herein in accordance with its art-accepted meaning.

By “ADF1-like” or “Adf1-like” is meant that a compound or an effect or action functionally resembles (mimics) the Adf1 transcription factor described herein or the effect or action of the Adf1 transcription factor. For example, “ADF1-like molecules” refer to proteins which functionally resemble (mimic) ADF1. ADF1-like molecules include the Adf1 transcription factor described herein. ADF1-like molecules need not have amino acid sequences analogous to the Adf1 transcription factor described herein. “Adf1-like genes” refer to genes that encode ADF1-like molecules and include the gene encoding the Adf1 transcription factor described herein. “ADF1-like activity” refers to biological activity and/or function that resembles (mimics) the biological activity or function of the Adf1 transcription factor described herein.

By “Adf1-dependent gene expression” is meant expression of downstream genes that contain the ADF1 binding site in their promoter. By “ADF1 binding site” is meant a sequence of DNA to which the Adf1 transcription factor described herein binds in order to activate or initiate transcription. In a particular embodiment, the Adf1 transcription factor described herein binds to the DNA sequence (consensus site) 5′-[G(C/T)(C/T)]_{3x or 4x}-3′ (England et al., J. Biol. Chem., 265(9):5086-5094 (1990)).

ADF1-like molecules can be intact protein or a functional or biologically active equivalent of an ADF1-like molecule. A functional or biologically active equivalent of an ADF1-like molecule refers to a molecule which functionally resembles (mimics) the Adf1 transcription factor described herein. For example, a functional equivalent of an ADF1-like molecule can contain a “SILENT” codon or one or more amino acid substitutions, deletions or additions (e.g., substitution of one acidic amino acid for another acidic amino acid; or substitution of one codon encoding the same or different hydrophobic amino acid for another codon encoding a hydrophobic amino acid). See Ausubel et al., eds., Current Protocols In Molecular Biology (New York: John Wiley & Sons) (1997). A functional equivalent of an ADF1-like molecule includes the Adf1 transcription factor described herein.

ADF1 analogs, or derivatives, are defined herein as proteins having amino acid sequences analogous to the Adf1 transcription factor described herein. Analogous amino acid sequences are defined herein to mean amino acid sequences with sufficient identity of amino acid sequence of the Adf1 transcription factor described herein to possess the biological activity of the Adf1 transcription factor, but with one or more “SILENT” changes in the amino acid sequence.

Biologically active ADF1-like fragments refer to biologically active polypeptide fragments of ADF1-like molecules and can include only a part of the full-length amino acid sequence of an ADF-like molecule, yet possess biological activity. Such fragments can be produced by carboxyl or amino terminal deletions, as well as one or more internal deletions.

Adf1-like fusion proteins comprise an ADF1-like molecule as described herein, referred to as a first moiety, linked to a second moiety not occurring in the ADF1-like molecule. The second moiety can be a single amino acid, peptide or polypeptide or other organic moiety, such as a carbohydrate, a lipid or an inorganic molecule.

The present invention further encompasses biologically active derivatives or analogs of ADF1 referred to herein as ADF1 peptide mimetics. These mimetics can be designed and produced by techniques known to those skilled in the art. See, e.g., U.S. Pat. Nos. 5,643,873 and 5,654,276. These mimetics are based on the Adf1 sequence, and peptide mimetics possess biological or functional activity similar to the biological activity or functional activity of the corresponding peptide compound, but possess a “biological advantage” over the corresponding peptide inhibitor with respect to one, or more, of the following properties: solubility, stability and susceptibility to hydrolysis and proteolysis.

Methods for preparing peptide mimetics include modifying the N-terminal amino group, the C-terminal carboxyl group and/or changing one or more of the amino linkages in the peptide to a non-amino linkage. Two or more such modifications can be coupled in one peptide mimetic inhibitor. Examples of modifications of peptides to produce peptide mimetics are described in U.S. Pat. Nos. 5,643,873 and 5,654,276.

The complete or partial nucleic acid or amino acid sequence of the Adf1 gene described herein and the novel antibodies to specific for ADF1 described herein and antigen-binding fragments thereof can be used, for example as a probe, to identify Adf1-like genes and ADF1-like molecules from a variety of animals, including vertebrates (e.g., mammals and particularly humans) and invertebrates, using methods known in the art. See, for example, Ausubel et al., eds., Current Protocols in Molecular Biology (New York: John Wiley & Sons) (1998); and Sambrook et al., Molecular Cloning: A Laboratory Manual, 2nd edition (New York: Cold Spring Harbor University Press) (1989).

Increased expression of Adf1-like molecules can be achieved by administration of an exogenous ADF1-like molecule or, alternatively, by increasing production of the endogenous ADF1-like molecule, for example by stimulating the endogenous gene to produce increased amounts of the ADF1-like molecule. In a particular embodiment, suitable pharmaceutical agents, as described herein, can be administered to the animal to stimulate the endogenous gene to produce increased amounts of a functional ADF1-like molecule, thereby increasing functional ADF1-like activity in the animal.

In some animals, the amount of ADF1-like molecule being produced can be of sufficient quantity, but the ADF1-like molecule is abnormal in some way and, thus, cannot exert its biological effect. That is, the ADF1-like molecule being produced has diminished or no functional activity. In this instance, providing copies of normal Adf1-like genes to the animal using techniques of gene transfer well known to those skilled in the art, can increase functional ADF1-like activity or concentration. Alternatively, an exogenous ADF1-like molecule, ADF1 analog, biologically active ADF1-like fragment or ADF1-like fusion protein can be administered to the animal.

By “enhancing CREB pathway function” or “enhances CREB pathway function” is meant the ability to enhance or improve CREB-dependent gene expression. CREB-dependent gene expression can be enhanced or improved by increasing endogenous CREB production, for example by directly or indirectly stimulating the endogenous gene to produce increased amounts of CREB, or by increasing functional (biologically active) CREB. See, e.g., U.S. Pat. No. 5,929,223; U.S. Pat. No. 6,051,559; and International Publication No. WO9611270 (published Apr. 18, 1996), which references are incorporated herein in their entirety by reference.

A compound which enhances CREB pathway function is defined herein as a compound with pharmacological activity and includes drugs, chemical compounds, ionic compounds, organic compounds, organic ligands, including cofactors, saccharides, recombinant and synthetic peptides, proteins, peptoids, nucleic acid sequences, including genes, nucleic acid products, and other molecules and compositions.

Compounds which enhance CREB pathway function can be exogenous CREB, CREB analogs, CREB-like molecules, biologically active CREB fragments, CREB fusion proteins, nucleic acid sequences encoding exogenous CREB, CREB analogs, CREB-like molecules, biologically active CREB fragments or CREB fusion proteins.

CREB analogs, or derivatives, are defined herein as proteins having amino acid sequences analogous to endogenous CREB. Analogous amino acid sequences are defined herein to mean amino acid sequences with sufficient identity of amino acid sequence of endogenous CREB to possess the biological activity of endogenous CREB, but with one or more “SILENT” changes in the amino acid sequence. CREB analogs include mammalian CREM, mammalian ATF-1 and other CREB/CREM/ATF-1 subfamily members.

CREB-like molecule, as the term is used herein, refers to a protein which functionally resembles (mimics) CREB. CREB-like molecules need not have amino acid sequences analogous to endogenous CREB.

Biologically active polypeptide fragments of CREB can include only a part of the full-length amino acid sequence of CREB, yet possess biological activity. Such fragments can be produced by carboxyl or amino terminal deletions, as well as internal deletions.

CREB fusion proteins comprise a CREB protein as described herein, referred to as a first moiety, linked to a second moiety not occurring in the CREB protein. The second moiety can be a single amino acid, peptide or polypeptide or other organic moiety, such as a carbohydrate, a lipid or an inorganic molecule.

Nucleic acid sequences are defined herein as heteropolymers of nucleic acid molecules. The nucleic acid molecules can be double stranded or single stranded and can be a deoxyribonucleotide (DNA) molecule, such as cDNA or genomic DNA, or a ribonucleotide (RNA) molecule. As such, the nucleic acid sequence can, for example, include one or more exons, with or without, as appropriate, introns, as well as one or more suitable control sequences. In one example, the nucleic acid molecule contains a single open reading frame which encodes a desired nucleic acid product. The nucleic acid sequence is “operably linked” to a suitable promoter.

A nucleic acid sequence encoding a desired ADF1-like molecule, ADF1 analog, biologically active ADF1-like fragment, ADF1-like fusion protein, ADF1 analog, ADF1-like fusion protein, CREB protein, CREB analog, CREB-like molecule or CREB fusion protein can be isolated from nature, modified from native sequences or manufactured de novo, as described in, for example, Ausubel et al., Current Protocols in Molecular Biology, (New York: John Wiley & Sons) (1998); and Sambrook et al., Molecular Cloning: A Laboratory Manual, 2nd edition (New York: Cold Spring Harbor University Press) (1989). Nucleic acids can be isolated and fused together by methods known in the art, such as exploiting and manufacturing compatible cloning or restriction sites.

Typically, the nucleic acid sequence will be a gene which encodes the desired ADF1-like molecule, ADF1 analog, ADF1-like fusion protein, CREB protein, CREB analog, CREB-like molecule or CREB fusion protein. Such a gene is typically operably linked to suitable control sequences capable of effecting the expression of the ADF1-like molecule or CREB protein, preferably in the CNS. The term “operably linked”, as used herein, is defined to mean that the gene (or the nucleic acid sequence) is linked to control sequences in a manner which allows expression of the gene (or the nucleic acid sequence). Generally, operably linked means contiguous.

Control sequences include a transcriptional promoter, an optional operator sequence to control transcription, a sequence encoding suitable messenger RNA (mRNA) ribosomal binding sites and sequences which control termination of transcription and translation. In a particular embodiment, a recombinant gene (or a nucleic acid sequence) encoding an ADF1-like molecule, ADF1 analog, biologically active ADF1-like fragment or ADF1-like fusion protein can be placed under the regulatory control of a promoter which can be induced or repressed, thereby offering a greater degree of control with respect to the level of the product. Similarly, a recombinant gene (or a nucleic acid sequence) encoding a CREB protein, CREB analog, CREB-like molecule, biologically active CREB fragment or CREB fusion protein can be placed under the regulatory control of a promoter which can be induced or repressed, thereby offering a greater degree of control with respect to the level of the product.

As used herein, the term “promoter” refers to a sequence of DNA, usually upstream (5′) of the coding region of a structural gene, which controls the expression of the coding region by providing recognition and binding sites for RNA polymerase and other factors which may be required for initiation of transcription. Suitable promoters are well known in the art. Exemplary promoters include the SV40 and human elongation factor (EFI) promoters. Other suitable promoters are readily available in the art (see, e.g., Ausubel et al., Current Protocols in Molecular Biology (New York: John Wiley & Sons, Inc.) (1998); Sambrook et al., Molecular Cloning: A Laboratory Manual, 2nd edition (New York: Cold Spring Harbor University Press (1989); and U.S. Pat. No. 5,681,735).

Pharmaceutical agents, as used herein, are compounds with pharmacological activity and include drugs, chemical compounds, ionic compounds, organic compounds, organic ligands, including cofactors, saccharides, recombinant and synthetic peptides, proteins, peptoids, nucleic acid sequences, including genes, nucleic acid products, and other molecules and compositions.

ADF1-like molecules, ADF1 analogs, biologically active ADF1-like fragments, ADF1 fusion proteins, CREB proteins, CREB analogs, CREB-like molecules, biologically active CREB fragments and CREB fusion proteins and pharmaceutical agents, as well as nucleic acid sequences encoding ADF1-like molecules, ADF1 analogs, biologically active ADF1-like fragments, ADF1-like fusion proteins, CREB proteins, CREB analogs, CREB-like molecules, biologically active CREB fragments or CREB fusion proteins, can be administered directly to an animal in a variety of ways. In a particular embodiment, administration is via transplant of neural tissue, e.g., by injecting neural cells into the brain. Other routes of administration are generally known in the art and include systematically, intravenous including infusion and/or bolus injection, intracerebroventricularly, intrathecal, parenteral, mucosal, implant, intraperitoneal, oral, intradermal, transdermal (e.g., in slow release polymers), intramuscular, subcutaneous, topical, epidural, etc. routes. Other suitable routes of administration can also be used, for example, to achieve absorption through epithelial or mucocutaneous linings. ADF1-like molecules, ADF1 analogs, biologically active ADF1-like fragments, ADF1 fusion proteins, CREB proteins, CREB analogs, CREB-like molecules, biologically active CREB fragments and CREB fusion proteins can also be administered by gene therapy, wherein a DNA molecule encoding a particular therapeutic protein or peptide is administered to the animal, e.g., via a vector, which causes the particular protein or peptide to be expressed and secreted at therapeutic levels in vivo.

A vector, as the term is used herein, refers to a nucleic acid vector, e.g., a DNA plasmid, virus or other suitable replicon (e.g., viral vector). Viral vectors include retrovirus, adenovirus, parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picomavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, 3rd Edition, B. N. Fields, et al., eds., Philadelphia, Pa.: Lippincott-Raven Publishers) (1996)). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., U.S. Pat. No. 5,801,030, the teachings of which are incorporated herein by reference.

A nucleic acid sequence encoding a protein or peptide (e.g., ADF1-like molecule, ADF1 analog, biologically active ADF1-like fragment, ADF1-like fusion protein, CREB protein, CREB analog, CREB-like molecule, biologically active CREB fragment and CREB fusion protein) can be inserted into a nucleic acid vector according to methods generally known in the art (see, e.g., Ausubel et al., eds., Current Protocols in Molecular Biology, John Wiley & Sons, Inc., New York (1998); Sambrook et al., eds., Molecular Cloning: A Laboratory Manual, 2nd edition (New York: Cold Spring Harbor University Press) (1989)).

The mode of administration is preferably at the location of the target cells. In a particular embodiment, the mode of administration is to cells of neural origin. Cells of neural origin include neural stem cells, neuroblastoma cells and neurons.

ADF1-like molecules, ADF1 analogs, biologically active ADF1-like fragments, ADF1 fusion proteins, CREB proteins, CREB analogs, CREB-like molecules, biologically active CREB fragments and CREB fusion proteins and pharmaceutical agents, as well as nucleic acid sequences encoding ADF1-like molecules, ADF1 analogs, biologically active ADF1-like fragments, ADF1-like fusion proteins, CREB proteins, CREB analogs, CREB-like molecules, biologically active CREB fragments or CREB fusion proteins, can be administered together with other components of biologically active agents, such as pharmaceutically acceptable surfactants (e.g., glycerides), excipients (e.g., lactose), stabilizers, preservatives, humectants, emollients, antioxidants, carriers, diluents and vehicles. If desired, certain sweetening, flavoring and/or coloring agents can also be added.

ADF1-like molecules, ADF1 analogs, biologically active ADF1-like fragments, ADF1 fusion proteins, CREB proteins, CREB analogs, CREB-like molecules, biologically active CREB fragments and CREB fusion proteins and pharmaceutical agents, as well as nucleic acid sequences encoding ADF1-like molecules, ADF1 analogs, biologically active ADF1-like fragments, ADF1-like fusion proteins, CREB proteins, CREB analogs, CREB-like molecules, biologically active CREB fragments or CREB fusion proteins, can be administered prophylactically or therapeutically to an animal prior to, simultaneously with or sequentially with other therapeutic regimens or agents (e.g., multiple drug regimens), including with other therapeutic regimens used for the treatment of long term memory defects or for the enhancement of long term memory formation. ADF1-like molecules, ADF1 analogs, biologically active ADF1-like fragments, ADF1 fusion proteins, CREB proteins, CREB analogs, CREB-like molecules, biologically active CREB fragments and CREB fusion proteins and pharmaceutical agents, as well as nucleic acid sequences encoding ADF1-like molecules, ADF1 analogs, biologically active ADF1-like fragments, ADF1-like fusion proteins, CREB proteins, CREB analogs, CREB-like molecules, biologically active CREB fragments or CREB fusion proteins, that are administered simultaneously with other therapeutic agents can be administered in the same or different compositions. Two or more different ADF1-like molecules, ADF1 analogs, biologically active ADF1-like fragments, ADF1 fusion proteins, CREB proteins, CREB analogs, CREB-like molecules, biologically active CREB fragments, CREB fusion proteins, nucleic acid sequences, pharmaceutical agents or combinations thereof can also be administered.

ADF1-like molecules, ADF1 analogs, biologically active ADF1-like fragments, ADF1 fusion proteins, CREB proteins, CREB analogs, CREB-like molecules, biologically active CREB fragments and CREB fusion proteins and pharmaceutical agents, as well as nucleic acid sequences encoding ADF1-like molecules, ADF1 analogs, biologically active ADF1-like fragments, ADF1-like fusion proteins, CREB proteins, CREB analogs, CREB-like molecules, biologically active CREB fragments or CREB fusion proteins, can be formulated as a solution, suspension, emulsion or lyophilized powder in association with a pharmaceutically acceptable parenteral vehicle. Examples of such vehicles are water, saline, Ringer's solution, isotonic sodium chloride solution, dextrose solution, and 5% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation can be sterilized by commonly used techniques. Suitable pharmaceutical carriers are described in Remington's Pharmaceutical Sciences.

An effective amount of ADF1-like molecule, ADF1 analog, biologically active ADF1-like fragment, ADF1 fusion protein, nucleic acid sequence or pharmaceutical agent is that amount, or dose, administered to an animal that is required to effect a change (increase or decrease) in expression of an Adf1-like gene or in functional ADF1-like activity. The dosage administered to an animal, including frequency of administration, will vary depending upon a variety of factors, including pharmacodynamic characteristics of the particular augmenting agent, mode and route of administration; size, age, sex, health, body weight and diet of the recipient; nature and extent of symptoms being treated or nature and extent of the cognitive function(s) being enhanced or modulated, kind of concurrent treatment, frequency of treatment, and the effect desired.

An effective amount of CREB protein, CREB analog, CREB-like molecule, biologically active CREB fragment, CREB fusion protein, nucleic acid sequence or pharmaceutical agent is that amount, or dose, administered to an animal that is required to effect a change (increase or decrease) in CREB-dependent gene expression, particularly in cells of neural origin. The dosage administered to an animal, including frequency of administration, will vary depending upon a variety of factors, including pharmacodynamic characteristics of the particular augmenting agent, mode and route of administration; size, age, sex, health, body weight and diet of the recipient; nature and extent of symptoms being treated or nature and extent of the cognitive function(s) being enhanced or modulated, kind of concurrent treatment, frequency of treatment, and the effect desired.

ADF1-like molecules, ADF1 analogs, biologically active ADF1-like fragments, ADF1 fusion proteins, CREB proteins, CREB analogs, CREB-like molecules, biologically active CREB fragments, CREB fusion proteins, nucleic acid sequences and pharmaceutical agents can be administered in single or divided doses (e.g., a series of doses separated by intervals of days, weeks or months), or in a sustained release form, depending upon factors such as nature and extent of symptoms, kind of concurrent treatment and the effect desired. Other therapeutic regimens or agents can be used in conjunction with the present invention. Adjustment and manipulation of established dosage ranges are well within the ability of those skilled in the art.

Once an effective amount has been administered, a maintenance amount of an ADF1-like molecule, ADF1 analog, biologically active ADF1-like fragment, ADF1 fusion protein, CREB protein, CREB analog, CREB-like molecule, biologically active CREB fragment, CREB fusion protein or pharmaceutical agent, or nucleic acid sequence encoding an ADF1-like molecule, ADF1 analog, biologically active ADF1-like fragment, ADF1 fusion protein, CREB protein, CREB analog, CREB-like molecule, biologically active CREB fragment or CREB fusion protein, can be administered to the animal. A maintenance amount is the amount of ADF1-like molecule, ADF1 analog, biologically active ADF1-like fragment, ADF1 fusion protein or pharmaceutical agent (or nucleic acid sequence encoding an ADF1-like molecule, ADF1 analog, biologically active ADF1-like fragment or ADF1 fusion protein) necessary to maintain the change (increase or decrease) in expression of an Adf1-like gene or in functional ADF1-like activity achieved by the effective dose. A maintenance amount is the amount of CREB protein, CREB analog, CREB-like molecule, biologically active CREB fragment, CREB fusion protein (or nucleic acid sequence encoding a CREB protein, CREB analog, CREB-like molecule, biologically active CREB fragment or CREB fusion protein) necessary to maintain the change in CREB-dependent gene expression achieved by the effective dose. The maintenance amount can be administered in the form of a single dose, or a series or doses separated by intervals of days or weeks (divided doses). Second or subsequent administrations can be administered at a dosage which is the same, less than or greater than the initial or previous dose administered to the animal. Determination of such amounts are well within the ability of those skilled in the art.

The present invention also relates to methods of screening a pharmaceutical agent for its ability to modulate ADF1-like activity in an animal, preferably an adult animal (particularly an animal (e.g., human, other mammal, vertebrate or invertebrate) with a defect in long term memory formation associated with a defect in an ADF1-like molecule) comprising (a) administering the pharmaceutical agent to the animal; and (b) determining the functional ADF1-like activity in the animal obtained in step (a) relative to the functional ADF1-like activity in a control animal to which the pharmaceutical has not been administered. A difference in the functional ADF1-like activity determined in the animal treated with the pharmaceutical agent relative to the functional ADF1-like activity determined in the control animal identifies the pharmaceutical agent as one having the ability to modulate ADF1-like activity in the animal.

The invention further relates to methods of screening a pharmaceutical agent for its ability to modulate long term memory formation in an animal, preferably an adult animal (particularly an animal (e.g., human, other mammal, vertebrate or invertebrate) with a defect in long term memory formation associated with a defect in an ADF1-like molecule) comprising (a) administering the pharmaceutical agent to the animal; (b) determining the functional ADF1-like activity in the animal obtained in step (a) relative to the functional ADF1-like activity in a control animal to which the pharmaceutical has not been administered; (c) selecting the animal in step (b) having a functional ADF1-like activity which differs from the functional ADF1-like activity in the control animal to which the pharmaceutical agent has not been administered; (d) training the animal selected in step (c) under conditions appropriate to produce long term memory formation in the animal; (e) assessing long term memory formation in the animal trained in step (d); and (f) comparing long term memory formation assessed in step (e) with long term memory formation produced in the control animal to which the pharmaceutical agent has not been administered. A difference in long term memory formation assessed in the animal treated with the pharmaceutical agent relative to the long term memory formation assessed in the control animal identifies the pharmaceutical agent as one which has the ability to modulate long term memory formation in the animal.

In another embodiment of screening a pharmaceutical agent for its ability to modulate long term memory formation, the method comprises (a) administering a pharmaceutical agent to an animal having an inducible Adf1-like gene; (b) inducing expression of the Adf1-like gene in the animal; (c) training the animal under conditions appropriate to produce long term memory formation in the animal; (d) assessing long term memory formation in the animal trained in step (c); and (e) comparing long term memory formation assessed in step (d) with long term memory formation produced in a control animal to which the pharmaceutical agent has not been administered. If a difference is noted in long term memory formation assessed in the animal treated with the pharmaceutical agent relative to the long term memory formation assessed in the control animal, the pharmaceutical agent can be categorized as having the ability to modulate long term memory formation in the animal.

The invention also relates to methods of assessing the effect of a pharmaceutical agent on ADF1-like activity in an animal, preferably an adult animal (particularly an animal (e.g., human, other mammal, vertebrate or invertebrate) with a defect in long term memory formation associated with a defect in an ADF1-like molecule) comprising (a) administering the pharmaceutical agent to the animal; and (b) determining the functional ADF1-like activity in the animal obtained in step (a) relative to the functional ADF1-like activity in a control animal to which the pharmaceutical has not been administered. A difference in the functional ADF1-like activity determined in the animal treated with the pharmaceutical agent relative to the functional ADF1-like activity determined in the control animal identifies the pharmaceutical agent as one having an effect on ADF1-like activity in the animal.

The invention further relates to methods for assessing the effect a pharmaceutical agent on long term memory formation in an animal, preferably an adult animal (particularly an animal (e.g., human, other mammal, vertebrate or invertebrate) with a defect in long term memory formation associated with a defect in an ADF1-like molecule) comprising (a) administering the pharmaceutical agent to the animal; (b) determining the functional ADF1-like activity in the animal obtained in step (a) relative to the functional ADF1-like activity in a control animal to which the pharmaceutical has not been administered; (c) selecting the animal in step (b) having a functional ADF1-like activity which differs from the functional ADF1-like activity in the control animal to which the pharmaceutical agent has not been administered; (d) training the animal selected in step (c) under conditions appropriate to produce long term memory formation in the animal; (e) assessing long term memory formation in the animal trained in step (d); and (f) comparing long term memory formation assessed in step (e) with long term memory formation produced in the control animal to which the pharmaceutical agent has not been administered. A difference in long term memory formation assessed in the animal treated with the pharmaceutical agent relative to the long term memory formation assessed in the control animal identifies the pharmaceutical agent as one having an effect on long term memory formation in the animal.

In another embodiment for assessing the effect a pharmaceutical agent on long term memory formation, the method comprises (a) administering a pharmaceutical agent to an animal having an inducible Adf1-like gene; (b) inducing expression of the Adf1-like gene in the animal; (c) training the animal under conditions appropriate to produce long term memory formation in the animal; (d) assessing long term memory formation in the animal trained in step (c); and (e) comparing long term memory formation assessed in step (d) with long term memory formation produced in a control animal to which the pharmaceutical agent has not been administered. If a difference is noted in long term memory formation assessed in the animal treated with the pharmaceutical agent relative to the long term memory formation assessed in the control animal, the pharmaceutical agent can be categorized as having an effect on long term memory formation in the animal.

Training of animals for long term memory formation are conducted using methods generally known in the art (see, e.g., Josselyn et al., Society for Neurosci., 24:926, Abstract 365.10 (1998); Casella and Davis, Physiol. Behav., 36:377-383 (1986); Guzowski et al., Proc. Natl. Acad. Sci. USA, 94:2693-2698 (1997); Lamprecht et al., J. Neuroscience, 17(21):6443-6450 (1997): Bourtchuladze et al., Cell, 79:59-68 (1994); Kogan et al., Curr. Biol., 7:1-11 (1996); Tully and Quinn, J. Comp. Physiol. A Sens. Neural. Behav. Physiol., 157:263-277 (1985); Tully et al., Cell, 79:35-47 (1994)).

Pharmaceutical agents, such as drugs, chemical compounds, ionic compounds, organic compounds, organic ligands, including cofactors, saccharides, recombinant and synthetic peptides, proteins, peptoids, nucleic acid sequences, including genes, nucleic acid products, and other molecules and compositions, can be individually screened or one or more pharmaceutical agent(s) can be tested simultaneously for the ability to modulate ADF1-like activity and/or the ability to modulate long term memory formation in accordance with the methods herein. Where a mixture of pharmaceutical agents is tested, the pharmaceutical agents selected by the methods described can be separated (as appropriate) and identified by suitable methods (e.g., chromatography, sequencing, PCR). The presence of one or more pharmaceutical agents in a test sample having the ability to modulate ADF1-like activity and/or the ability to modulate long term memory formation can also be determined according to these methods.

Large combinatorial libraries of pharmaceutical agents (e.g., organic compounds, recombinant or synthetic peptides, peptoids, nucleic acids) produced by combinatorial chemical synthesis or other methods can be tested (see e.g., Zuckerman, R. N. et al., J. Med. Chem., 37:2678-2685 (1994) and references cited therein; see also, Ohlmeyer, M. H. J. et al., Proc. Natl. Acad. Sci. USA, 90:10922-10926 (1993) and DeWitt, S. H. et al., Proc. Natl. Acad. Sci. USA, 90:6909-6913 (1993), relating to tagged compounds; Rutter, W. J. et al. U.S. Pat. No. 5,010,175; Huebner, V. D. et al., U.S. Pat. No. 5,182,366; and Geysen, H. M., U.S. Pat. No. 4,833,092). The teachings of these references are incorporated herein by reference. Where pharmaceutical agents selected from a combinatorial library carry unique tags, identification of individual pharmaceutical agents by chromatographic methods is possible.

Chemical libraries, microbial broths and phage display libraries can also be tested (screened) for the presence of one or more pharmaceutical agent(s) which is capable of modulating ADF1-like activity and/or modulating long term memory formation in accordance with the methods herein.

The present invention also relates to methods of screening for or identifying a pharmaceutical agent which is capable of modulating ADF1-like activity comprising (a) introducing into host cells (particularly cells of neural origin) a DNA construct, wherein the DNA construct comprises (1) DNA encoding an indicator gene and (2) a promoter sequence which comprises an ADF1 binding site and is operably linked to the DNA encoding the indicator gene; (b) producing a sample by introducing into host cells comprising the DNA construct a pharmaceutical agent to be assessed for its ability to modulate ADF1-like activity under conditions appropriate for expression of the indicator gene; (c) detecting expression of the indicator gene in the sample obtained in step (b); and (d) comparing expression detected in step (c) with expression of the indicator gene detected in control cells into which the pharmaceutical agent has not been introduced. A difference in expression of indicator gene in the sample obtained in step b) compared to the expression of indicator gene in control cells identifies the pharmaceutical agent as one which modulates ADF1-like activity.

The term “indicator gene”, as used herein, refers to a nucleic acid sequence whose product can be easily assayed, for example, colorimetrically as an enzymatic reaction product, such as the lacZ gene which encodes for β-galactosidase. Other examples of widely used indicator genes include those encoding enzymes, such as β-glucoronidase and β-glucosidase; luminescent molecules, such as green flourescent protein and firefly luciferase; and auxotrophic markers such, as His3p and Ura3p. See, e.g., Ausubel et al., Current Protocols In Molecular Biology (New York: John Wiley & Sons, Inc.), Chapter 9 (1998)).

A promoter sequence which comprises an ADF1 binding site can comprise one or more than one (i.e., multiple) ADF1 binding sites.

DNA constructs comprising DNA encoding an indicator gene operably linked to a promoter sequence comprising one or multiple ADF1 binding sites can be manufactured as described in, for example, Ausubel et al., Current Protocols In Molecular Biology (New York: John Wiley & Sons) (1998); and Sambrook et al., Molecular Cloning: A Laboratory Manual, 2nd edition (New York: Cold Spring Harbor University Press (1989). DNA constructs can be introduced into cells according to methods known in the art (e.g., transformation, direct uptake, calcium phosphate precipitation, electroporation, projectile bombardment, using liposomes). Such methods are described in more detail, for example, in Sambrook et al., Molecular Cloning: A Laboratory Manual, 2nd edition (New York: Cold Spring Harbor University Press) (1989); and Ausubel, et al., Current Protocols in Molecular Biology (New York: John Wiley & Sons) (1998).

As used herein, a cell refers to an animal cell. The cell can be a stem cell or somatic cell. Suitable animal cells can be of, for example, mammalian origin. Examples of mammalian cells include human (such as HeLa cells), bovine, ovine, porcine, murine (such as embryonic stem cells), rabbit and monkey (such as COS1 cells) cells. Preferably, the cell is of neural origin (such as a neuroblastoma, neuron, neural stem cell, etc.). The cell can also be an embryonic cell, bone marrow stem cell or other progenitor cell. Where the cell is a somatic cell, the cell can be, for example, an epithelial cell, fibroblast, smooth muscle cell, blood cell (including a hematopoietic cell, red blood cell, T-cell, B-cell, etc.), tumor cell, cardiac muscle cell, macrophage, dendritic cell, neuronal cell (e.g., a glial cell or astrocyte), or pathogen-infected cell (e.g., those infected by bacteria, viruses, virusoids, parasites, or prions).

The cells can be obtained commercially or from a depository or obtained directly from an animal, such as by biopsy.

Pharmaceutical agents identified in accordance with the screening methods herein can be administered to an animal with a defect in long term memory formation associated with a defect in an ADF1-like molecule or a defect in synaptic plasticity associated with a defect in an ADF1-like molecule in the treatment of the animal in accordance with the methods herein. Pharmaceutical agents identified in accordance with the screening methods herein can also be administered to an animal to modulate or enhance long term memory formation or synaptic plasticity in accordance with the methods herein.

The present invention further relates to the novel antibodies (immunoglobulins) described herein specific for ADF1 and antigen-binding fragments thereof. The antibodies of the invention can be polyclonal or monoclonal, and the term “antibody” is intended to encompass both polyclonal and monoclonal antibodies. The terms polyclonal and monoclonal refer to the degree of homogeneity of an antibody preparation, and are not intended to be limited to particular methods of production. The term “antibody”, as used herein, also encompasses functional fragments of antibodies, including fragments of human, chimeric, humanized, primatized, veneered or single chain antibodies. Functional fragments include antigen-binding fragments specific for ADF1. Antigen-binding fragments specific for ADF1 include, but are not limited to, Fab, Fab′, F(ab′)₂ and Fv fragments. Such fragments can be produced by enzymatic cleavage or recombinant techniques. For example, papain or pepsin cleavage can generate Fab or F(ab′)₂ fragments, respectively. Other proteases with the requisite substrate specificity can also be used to generare Fab or F(ab′)₂ fragments. Antibodies can also be produced in a variety of truncated forms using antibody genes in which one or more stop codons has been introduced upstream of the natural stop site. For example, a chimeric gene encoding a F(ab′)₂ heavy chain portion can be designed to include DNA sequences encoding the CH₁ domain and hinge region of the heavy chain.

Single chain antibodies, and chimeric, humanized or primatized (CDR-grafted), or veneered antibodies, as well as chimeric, CDR-grafted or veneered single chain antibodies, comprising portions derived from different species, and the like are also encompassed by the present invention and the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0 125 023 B1; Boss et al., U.S. Pat. No. 4,816,397; Boss et al., European Patent No. 0 120 694 B1; Neuberger et al., International Publication No. WO86/01533; Neuberger et al., European Patent No. 0 194 276 B1; Winter et al., U.S. Pat. No. 5,225,539; Winter et al., European Patent No. 0 239 400 B1; Queen et al., European Patent No.0 451 216 B1; and Padlan et al., EP 0 519 596 A1. See also, Newman et al., BioTechnology, 10:1455-1460 (1992), regarding primatized antibody, and Ladner et al., U.S. Pat. No. 4,946,778 and Bird et al., Science, 242:423-426 (1988)) regarding single chain antibodies.

An “antigen” is a molecule or a portion of a molecule capable of being bound by an antibody which is additionally capable of inducing an animal to produce antibody capable of binding to an epitope of that antigen. An antigen can have one or more than one epitope.

The term “epitope” is meant to refer to that portion of the antigen capable of being recognized by and bound by an antibody at one or more of the antibody's antigen binding region. Epitopes usually consist of chemically active surface groupings of molecules such as amino acids or sugar side chains and have specific three dimensional structural characteristics as well as specific charge characteristics.

As used herein, the term “specific” when referring to an antibody-antigen interaction is used to indicate that the antibody can selectively bind to ADF1.

The invention also relates to an isolated cell which produces a novel antibody or antigen-binding fragment of the invention. In a preferred embodiment, the isolated antibody-producing cell of the invention is an immortalized cell, such as a hybridoma, heterohybridoma, lymphoblastoid cell or a recombinant cell. The antibody-producing cells of the present invention have uses other than for the production of antibodies. For example, the cell of the present invention can be fused with other cells (such as suitably drug-marked human myeloma, mouse myeloma, human-mouse heteromyeloma or human lymphoblastoid cells) to produce, for example, additional hybridomas, and thus provide for the transfer of the genes encoding the antibody. In addition, the cell can be used as a source of nucleic acids encoding the immunoglobulin chains of the antibodies of the invention, which can be isolated and expressed (e.g., upon transfer to other cells using any suitable techniques (see, e.g., Cabilly et al., U.S. Pat. No. 4,816,567; and Winter, U.S. Pat. No. 5,225,535)). For instance, clones comprising a sequence encoding a rearranged light and/or heavy chain of an antibody of the invention can be isolated (e.g., by polymerase chain reaction (PCR)) or complementary DNA (cDNA) libraries can be prepared from messenger RNA (mRNA) isolated from the cell lines, and cDNA clones encoding an immunoglobulin chain(s) of an antibody of the invention can be isolated. Thus, nucleic acids encoding the heavy and/or light chains of the antibodies or portions thereof can be obtained and used for the production of the specific immunoglobulin, immunoglobulin chain or variants thereof (e.g., humanized immunoglobulins) in a variety of host cells or in an in vivo translation system. For example, the nucleic acids, including cDNAs, or derivatives thereof encoding variants such as a humanized immunoglobulin or immunoglobulin chain, can be placed into suitable prokaryotic or eukaryotic vectors (e.g., expression vectors) and introduced into a suitable host cell by an appropriate method (e.g., transformation, transfection, electroporation, infection) such that the nucleic acid is operably linked to one or more expression control elements (e.g., in the vector or integrated into the host cell genome) to produce a recombinant antibody-producing cell.

As described above, the novel antibodies and antigen-binding fragments of the present invention can be used, for example as a probe, to identify Adf1-like genes and ADF1-like molecules from a variety of animals, including vertebrates (e.g., mammals and particularly humans) and invertebrates, using methods known in the art. Such methods are described, for example, in Ausubel et al., Current Protocols in Molecular Biology (New York: John Wiley & Sons (1998); and Sambrook et al., Molecular Cloning: A Laboratory Manual, 2nd edition (New York: Cold Spring Harbor University Press) (1989).

The novel antibodies of the present invention have application in procedures in which Adf1 and Adf1-like expression can be detected in cells. For example, the antibodies and antigen-binding fragments can be used to detect and/or quantify the level of ADF1-like activity in cells according to methods generally known in the art (e.g., immunoassays (e.g., sandwich assays, competitive immunoassays (e.g., radioimmunoassay (RIA), enzyme-linked immunoadsorbant assay (ELISA)), flow cytometry (e.g., FACS analysis), immunohistology, immunoblot (e.g., Western blot)).

The antibodies and antigen-binding fragments also have application in screening methods as a readout of Adf1 and Adf1-like expression levels.

The antibodies and antigen-binding fragments of the present invention can be used to identify proteins which interact with or bind to the Adf1 transcription factor described herein using methods known in the art (e.g., co-immunoprecipitation assays) (see, e.g., Ausubel et al., Current Protocols in Molecular Biology (New York: John Wiley & Sons) (1998); and Harlow and Lane, Antibodies: A Laboratory Manual (Plainview, N.Y.: Cold Spring Harbor Laboratory Press) (1998)). The antibodies and antigen-binding fragments can further be used to identify other DNA sequences to which the Adf1 transcription factor described herein binds in order to activate or initiate transcription (i.e., to identify other ADF1 binding sites) using methods known in the art (e.g., gel mobility shift assays (also referred to as gel retardation assays, band retardation assays, gel mobility shift DNA-binding assays), antibody supershift assays) (see, e.g., Ausubel et al., Current Protocols in Molecular Biology (New York: John Wiley & Sons) (1998)).

The present invention will now be illustrated by the following examples, which are not to be considered limiting in any way.

### EXAMPLES

The following materials and methods were used in the work described in the Examples.

### Isolation of nalyot^{P1} Mutant

An X-linked PlacW transposon (Bier et al., Science, 240(4854):913-916 (1988)) was mobilized to generate 2,182 transposant strains with independent insertions on the second and third chromosomes (cf. Boynton and Tully, Genetics, 131:655-672 (1992); Dura et al., J. Neurogent., 9:1-14 (1993)). Three-hour memory after a single training session of Pavlovian olfactory learning was quantified with one performance index (PI) for each of these transposant strains. n=4 PIs then were generated for those strains that scored 70% or less of a wild-type parental strain [w¹¹¹⁸ (CS10)]. At this stage of the screen, 93 mutant strains yielded mean three-hour memory scores 70% wild-type or less. Each of these candidate mutant strains then was outcrossed for five generations to the parental strain to equilibrate their (heterogeneous) genetic backgrounds. When three-hour memory again was quantified (n=4 PIs) in these outcrossed strains, only eight of the 93 candidate mutants still yielded mean scores <70% wildtype. Finally, “task relevant” sensorimotor tasks were assayed in these eight mutant strains. All eight showed normal shock reactivity; four, G_{B}335 and E_{J}51, E_{J}220 and E_{S}152, showed significantly reduced olfactory acuity (Boynton and Tully, Genetics, 131:655-672 (1992); Dura et al., J. Neurogent., 9:1-14 (1993). [G_{B}335, now named dare, has been studied further and shows preferential expression in antenna (Freeman et al., Development, 126:4591-4602 (1999)]. The remaining four mutant strains displayed normal sensorimotor responses and were named latheo (Boynton and Tully, Genetics, 131:655-672 (1992); Pinto et al., Neuron, 23:45-54 (1999); Rohrbough et al., Neuron, 23:55-70 (1999), linotte (Dura et al., J. Neurogent., 9:1-14 (1993); Bolwig et al., Neuron, 15:829-842 (1995); Simon et al., Mech. Dev., 76:42-55 (1998), golovan and nalyot.

### Cloning and Characterization of nalyot Genomic Region

The PlacW transposon includes a unique Sac II restriction site followed by the bacterial origin of replication and ampicillin resistance gene (FIG. 2A). Digestion of nalyot (nal) genomic DNA with SacII ligation under dilute conditions and bacterial transformation allowed plasmid rescue of a 9.4 kb Sac II restriction fragment along with flanking DNA from the genomic region. Chromosome in situs and Southern blotting experiments verified that this fragment co-mapped to the P insertion site. The radiolabeled rescue fragments were used to screen one million plaques of a lambda-DashII Drosophila Can-S genomic library (Stratagene). Isolation, subcloning and restriction analysis of 10 independent genomic clones led to the construction of a 35 kb map spanning the genomic region around the P element insertion site (FIG. 2A).

Intron/exon maps of the Adf1 and cn20 transcription units are shown in FIG. 2A. The nal^{P1} element (arrow) is inserted within an intron of the Adf1 transcription unit, 147 bp downstream of the splice donor site. The Adf1 gene encodes a transcription factor distantly related to the myb family (England et al., Proc. Natl. Acad. Sci. USA, 89:683-687 (1992)) and is alternatively spliced into (at least) two mRNAs. i1 and i2 correspond to two potential translation start sites. Two additional introns of about 3.5 kb and 59 bp appear to be spliced constitutively and separate the remainder of the Adf1 transcription unit into 274 bp and 1,013 bp exons. The cn20 gene is novel and produces a single, unspliced transcript that can encode a 395 amino acid protein. The extent of the genomic deletion in nal^{le60} is indicated. Restriction sites: B, Bam HI; E, Eco RI; H, Hind III; S, Sac II.

### Isolation and Characterization of cDNAs

Selected genomic DNA probes were hybridized to two Drosophila adult head cDNA libraries, a lambda gt11 bacteriophage adult head library (Salvaterra) and a pJG4-5 plasmid library (Roshbash). From these two libraries, eleven cDNA clones, corresponding to two independent transcription units, were isolated and evaluated by restriction analysis. Ten clones corresponded to the Adf1 transcription unit. Restriction-mapping and sequence analysis of a subset of these revealed a common 3′ end processing site and heterogeneity at the 5′ end. The 5′ heterogeneity reflected the partial splicing of intron 1 (114 bp) and, perhaps, incomplete first strand synthesis. One clone corresponded to cn20, an independent, neighboring transcription unit.

### Northern Blot Analysis and RNA Quantification

Wild-type (or mutant) total RNA from whole adult flies, adult heads, or adult bodies was isolated with the TriZOL reagent (BRL). The poly(A) fraction was subsequently purified with oligo(dT) cellulose (Collaborative Research) or magnetized oligo(dT) beads (Dyna1). Purified poly(A) RNA was fractionated by formaldehyde-agarose gel electrophoresis and transferred to a ZetaProbe nylon membrane (BioRad) in 10×SSC. The RNA on the dried membrane was fixed by UV-crosslinking at 2,500 ujoules (Stratalinker, Stratagene). For initial identification of transcript classes, membrane strips were probed overnight with radiolabeled genomic DNA fragments in high stringency Church and Gilbert Buffer, washed extensively and exposed to Kodak BioMax film. Each lane of each northern blot corresponds to 5 mg of poly(A) RNA.

To quantify the effect of the P-insertion on Adf1 and cn20 RNA levels, each blot was sequentially hybridized to Adf1, cn20, or rp49 ³²P-labeled probes and the resulting signals quantified with the Fujix Bas 1000 phosphorimager system (Fuji Photo Film Co., Ltd). Specifically, each blot was exposed to a phosphorimager screen, the screen was then scanned with Image Reader v1.4E, and the captures signals were analyzed and quantified with the Image Gauge v3.0 program. For each measurement, a signal of equivalent area was determined for each gene and a flanking region in the same lane, allowing normalization of the gene specific signal to the background signal. The values of Adf1 and cn20 were each normalized to control rp49 signal across different extracts or genotypes.

The results for Adf1 are: (1) adult head extracts: 1633±49 (wild-type) vs. 775±51 (nal), n=12 (triple determination of 4 independent extracts); (2) adult body extracts; 239±5 (wild-type) vs. 125±5 (nao), n=2. The results for cn20 are: (1) adult head extracts: 187±4 (wild-type) vs. 214±4 (nal), n=2; (2) adult body extracts; 373±9 (wild-type) vs. 386±9 (nal), n=2. Thus, relative to control levels of rp49 RNA, cn20 mRNA expression levels in both heads and bodies were similar in wildtype and mutant flies. In contrast, Adf1 mRNA expression levels were reduced by at least two-fold in mutant heads and bodies.

### RT-PCR Analysis of Adf1 RNA Splicing

To analyze Adf1 RNA splicing in nal^{P1} and wild-type flies, we carried out first-strand cDNA synthesis on 3 μg of poly(A) mRNA with an oligo(dT) primer, using the Promega Reverse Transcription System. One-fifth of the reaction then was used for each PCR reaction, which was performed with either a primer pair flanking the first exon (C-ADF1: 5′-CGACTGAGCTGGGACGTACC-3′ (SEQ ID NO:1); and C-ADF2: 5′-GGGCCTTGCGCACAAAGTGC-3′ (SEQ ID NO:2)) or a primer pair flanking the second exon (C-ADF3: 5′-TGCGCAAGGCCCAGACCTGG-3′ (SEQ ID NO:3); and C-ADF4: 5′-CACTGCGCTCTCGCTTGAGC-3′ (SEQ ID NO:4)). Each reaction was carried with KlenTaq DNA polymerase (Clontech), allowing hot-start conditions, and included 0.5 μl of ³²P-dCTP label. Amplification parameters were identical for the two primer pairs: 94° C. for 2.5 minutes followed by 35 cycles, each consisting of 30-seconds at 94° C. and 3-minutes at 68° C.

Amplification with the first primer pair yielded two expected products of 430 and 316 bp, corresponding to the retention and splicing of intron 1, respectively. To evaluate the processing of intron 1, 5 μl samples were removed after 25, 30 and 35 cycles. The products were resolved by polyacrylamide gel electrophoresis (PAGE) and the ratio of unspliced to spliced RNA determined by exposure of the gel to a phosphorimager. Amplification with the second primer pair yielded a single expected product of 441 bp, corresponding to splicing of intron 2. Neither retention of this intron in wild-type flies nor aberrant splicing of this intron in nal^{P1} mutants were observed, which was consistent with the absence of aberrantly-sized mRNA species on Northern blots.

### Excision Alleles of nal^{P1}

Excisions of the original nal^{P1} P-element insertion were generated as described in Dura et al. (J. Neurogenet., 9:1-14 (1993)) and were detected by loss of the mini-white sequence within PlacW. Fifty independent excision strains were isolated. Approximately one-third were sub-viable or lethal.

### PCR Characterization of Excision Alleles

Three primers were used to screen this panel of excisions. One primer, lat-LTR (5′-ACGGGACCACCTTATGTTAT-3′ (SEQ ID NO:5)) recognizes the P element LTRs, extending from −29 to −9, relative to each end of the P-element. The other two primers were specific for the nal genomic region. JIM-2N (5′-CTTGAGCAGCAGTTTGATCTC-3′ (SEQ ID NO:6)) was located 300 bp to the left (distal) of the P-element, and JIM-3 (5′-TAGAGCAGATCTTTACAGATAG-3′ (SEQ ID NO:7)) was located 222 bp to the right of the P-element (FIG. 2A). By performing a 3-primer reaction on each excision allele, the following were distinguished: (i) precise excisions, (ii) imprecise excisions wholly contained within the genomic region flanked by the PCR primers and (iii) deletions of enough flanking genomic sequence to eliminate the primer site. PCR conditions for each 50 μl diagnostic reaction (200 ng genomic DNA, 120 ng of each primer (˜10 μM), 0.2 mM dNTPs, 1×PCR Buffer, and 2.5 units Taq DNA polymerase (Perkin Elmer)) were one cycle at 94° C. for 2-minutes followed by 35 cycles, each consisting of 45-seconds at 94° C., 45-seconds at 55° C. and 90-seconds at 72° C.

Among the lethal excision alleles were two imprecise excisions (nal^{le60} and nal^{le55}) extending into flanking genomic DNA. For nal^{le55}, it was determined by quantitative southern analysis that at least 13 kb of genomic sequence from the original P-element insertion site to a point distal of the rightmost Eco RI site, was missing (FIG. 2B). For nal^{le60}, it was determined by quantitative southern analysis that the deletion was not contiguous with the P element but mapped between the left-proximal Eco RI and Bam HI sites.

The TaqPlus Long PCR System (Stratagene) was used to amplify out the wild-type and mutant genomic fragments spanning the deletion. The primers used were BAM-46-3 (5′-CAAGTGTCGTTGGCAAAGTGG-3′ (SEQ ID NO:8)) and C-ADF-3 (5′-TGCGCAAGGCCCAGACCTGG-3′ (SEQ ID NO:3)). PCR reactions were performed under high salt conditions as specified by the manufacturer. The optimized cycling parameters were one 45 minute cycle at 95° C. followed by 35 cycles, each consisting of 30 seconds at 95° C., 45 seconds at 70° C. and 7.5 minutes at 72° C. Detailed restriction analysis and sequencing of the cloned fragments revealed that nal^{le60} is a simple deletion, extending from approximately 300 bp upstream of the major Adf1 exon to 150 bp past it (FIG. 2B). In a separate analysis, the region spanning the original P-insertion in nal^{le60} was cloned and sequenced. It was discovered that the region retained an additional 38 bp (5′-CATGATGAAATAAACATGTTATTTCATCATGACGACGAC-3′ (SEQ ID NO:9)), consisting of 30 bp of the P-element termini (underlined) plus an 8 bp duplication of the genomic target site. Homozygous-viable excision, nal^{ve48}, was found to contain this same 38 bp perturbation with no additional disruptions in surrounding Adf1 genomic region.

### Generation of Transgenic Lines

To construct a heat-inducible version of Adf1, a 1,603 bp cDNA (c16) encoding the entire Adf1 open reading frame was inserted into the Eco RI site of pCaSpeR-hs. This fragment spans the region from 133 bp upstream of the start codon to 709 bp downstream of the stop codon. Germline transformation of w¹¹¹⁸ (CJiso1) parental flies was accomplished by microinjection, as previously described (Yin et al., Cell, 81:107-115 (1995); Spradling and Rubin, Science, 218:341-347 (1982); Rubin and Spradling, Science, 218:348-353 (1982)). Three transgenic strains were recovered: hsp-Adf1⁺-8 (X-linked), hsp-Adf1⁺-32 (2nd chromosome) and hsp-Adf1⁺-11 (third chromosome).

To construct a GAL4-inducible form of Adf1, a 1,256 bp EcoRI-Xba1 fragment from Adf1 (c16) was inserted into the Eco RI and Xba I sites of pUAST (Brand and Perrimon, Development, 118:401-415 (1993)). This fragment was identical to that used in construction of the hsp-Adf1 lines, except that it included 347 bp of sequence downstream of the stop codon. Three UAS-transgene lines were recovered (UAS-Adf1⁺-201B, UAS-Adf1⁺-202 and UAS-Adf1⁺-203).

### Antibody Production

The entire ADF1 open reading frame was inserted as a C-terminal fusion into the pET30(a) expression vector (Novagen). Robust IPTG induction of ADF1 fusion protein was obtained in transformed BL21 bacteria. The insoluble inclusion body fraction, isolated 3 hours after induction, was enriched nearly 85% for the ADF1 fusion protein, washed with PBS and used directly as antigen.

Mouse polyclonal and monoclonal antibodies were obtained by standard procedures (Harlow and Lane, Antibodies. A Laboratory Manual, Cold Spring Harbor Laboratory Press, Plainview, N.Y. (1998)). Three mice were inoculated with 50 μg of the inclusion body fraction (in complete Freund's adjuvant), then boosted every two weeks with 50 μg of inclusion body fraction in incomplete Freund's adjuvant. All three mice showed robust immune responses; one was sacrificed for hybridoma fusions. Of 800 candidate hybridoma lines, 17 showed a response in ADF1 dot-blot analyses. Subsequent evaluation of the 17 lines by western blotting and immunochemical assays led to the isolation of ten monoclonal lines, including MAb Adf1-8 and MAb Adf1- 17.

### Western Blot Analysis of ADF-1 Protein Levels in Mutant nal^{P1} and Wildtype Flies

Extracts were prepared from frozen heads or bodies as previously described (Yin et al., Cell, 79:49-58 (1994)). Protein concentrations were determined by the Bio-Rad protein assay. Protein samples were denatured in standard loading dye, separated by SDS-PAGE and transferred electrophoretically at 100 mA for 2 hours to a nitrocellulose membrane (Bio-Rad). Each membrane was blocked overnight at 4° C. in PBST+5% milk, then incubated for 1-2 hours with primary antibody in PBST+5% milk. Primary antibodies used were mouse polyclonal sera against ADF1 (1:1000), MAb Adf1-17 supernatant (1:20), monoclonal supernatant against TBP (1:5), and monoclonal ascites against α-tubulin (1:50,000) (Sigma). The membrane was washed in PBST and incubated for 1-2 hours with HRP-conjugated anti-IgG secondary antibody (Bio-Rad; 1:500). Following washing in PBST, products were visualized by enhanced chemiluminescence (Pierce SuperSignal ULTRA Substrate) and autoradiography.

### In situ Expression of Adf1

Fixation and staining of embryos and the third instar central nervous system (CNS) were carried out by standard procedures (DeZazzo et al., J. Neurosci., 19:8740-8746 (1999); Estes et al., J. Neurosci., 16:5443-5456 (1996)). Adult head sections were generated as described in Pinto et al. (Neuron, 23:45-54 (1999)). Briefly, wandering third instar were opened dorsally, trachea and fat body removed, and fixed for one hour in 3.5% paraformaldehyde in C²⁺-free PBS containing 0.5 mM EGTA and 0.2 mM MgCl₂ and 8% Triton X-100. The CNS, salivary glands and parts of the gut were left attached to the body wall until mounting. After fixation, preparations were washed with normal PBS containing 0.15% Triton X-100 (PBT), followed by a 10-minute incubation in PBT containing 25 mM glycine. Tissue was blocked for 0.5-1 hour in PBT containing 2% BSA and 5% normal goat serum before incubation for 2 hour in MAb Adf1-17, diluted 1:50 in PBT blocking solution. The secondary antibody was either peroxidase-conjugated goat anti-mouse IgG+IgM antibody (diluted 1:500 in PBT blocking solution) (Jackson ImmunoResearch) or Texas red conjugated goat-anti-mouse antibody (diluted 1:200 in PBT blocking solution) (ICN, Costa Mesa, Calif.). Preparations were washed 3 times, 15 minutes each, with PBT blocking solution between antibody incubations. After incubations with secondary antibody, the tissue was washed with PBT and PBS and mounted in polyvinyl alcohol.

Images of the embryo and adult head were captured digitally with the Spot CCD Camera (Diagnostic Instruments, Inc) under bright field optics on a Zeiss Axiophot Microscope. Images of the third instar CNS were captured digitally with a Nikon PCM-2000 laser-scanning confocal microscope.

### Planimetry

Flies were prepared for mass histology as previously described (Heisenberg and Böhl, Z. Naturforsch, 34c:143-147 (1979)). All genotypes were represented in equal numbers in each preparation. Frontal paraffin sections (7 um) were viewed under a fluorescence microscope. Entire brains of mutant flies were inspected for obvious defects in gross anatomy. Brain neuropilar substructure volumes were derived from images captured by a digitized camera and fed to a personal computer. Serial sections were traced using a digitized tablet and volumetric integration was performed by a planimetry program (de Belle and Heisenberg, Proc. Natl. Acad. Sci. USA, 93:9875-9880 (1996); Bolwig et al., Neuron, 15:829-842 (1995); Heisenberg et al., J. Neurosci., 15:1851-1960 (1995)).

### Neuromuscular Junction Morphology

Synaptic terminals were visualized by incubating body walls first with rabbit anti-synaptotagmin (1:200), and then Texas red-conjugated anti-rabbit antibody (1:200; ICN, Costa Mesa, Calif.). Following staining, muscles 6 and 7 in abdominal segment 2 (A2) were imaged with a BioRad 600 or Nikon PCM-2000 laser-scanning confocal microscope. To avoid experimental bias, the arbor on the right side of the animal was examined, unless it was unusable due to damage or distortion. Terminal boutons were visualized with a 60×objective using 1 μm optical sections. To measure muscle area, muscles 6 and 7 were also imaged at 20×.

Synaptic boutons of a given terminal arbor were counted from a projection of all optical sections in a 60×confocal image, using Metamorph Software (Universal Imaging, West Chester, Pa.). Images were coded and analyzed in a “blind” manner. Synaptotagmin is expressed in synaptic vesicles and localized to discrete variocosities, making quantification of arbor size straightforward (Davis et al., Neuron, 17:669-679 (1996)). No attempt was made to separate type 1s and type 1b boutons. Type 2 boutons occurred only rarely on muscles 6 and 7 in segment A2 in any genotype and were not included in this analysis. Because the number of synaptic boutons is strongly correlated with the size of the muscle (Broadie and Bate, Neuron, 11:607-619 (1993)), data were corrected by dividing by the surface area of the muscle measured from a 20×image and then normalized to the wildtype.

### Neuromuscular Junction Electrophysiology

Synaptic currents were recorded from muscle 6 in segment A2 using two-electrode voltage clamp as described previously (Stimson et al., J. Neurosci., 18:9638-9649 (1998)). All procedures were performed at a room temperature of 18° C. Larvae were dissected in calcium-free HL3 saline, containing 70 mM NaCl, 5 mM KCl, 20 mM MgCl₂, 10 mM NaHCO₃, 5 mM trehalose, 155 mM sucrose, 5 mM HEPES and 0.5 mM EGTA, pH 7.3. The CNS was removed to eliminate endogenous motoneuron activity. Recordings were performed in “normal” HL3 saline, which was identical to the above except for the presence of 1.5 mM CaCl₂ and the absence of EGTA. The segmental nerve, which carries the axons of the two motoneurons innervating muscle 6 (Broadie and Bate, Neuron, 11:607-619 (1993)), was stimulated via a glass-tipped suction electrode. Stimuli consisted of 0.2 msec pulses, at a voltage approximately twice that required to evoke a compound response, generated by an isolated pulse stimulator (AM Systems model 2100, Everett, Wash.) gated by pClamp6 software (Axon Instruments, Foster City, Calif.).

Intracellular electrodes were pulled from thin-walled borosilicate glass and filled with either 3 M KCl (voltage electrode, Rₑ=15-22 MΩ). All data were acquired in two-electrode voltage clamp, at a Vₘ of −70 mV, using an Axoclamp 2B amplifier (Axon Instruments) in conjunction with pClamp6 software on a personal computer. A preparation was considered acceptable based on two criteria: resting Vₘ>−50 mV in current clamp after impalement with both electrodes, and Rᵢₙ>2 MΩ in voltage clamp. Data were filtered at 1 kHz using pClamp software before analysis.

EJC amplitude was determined from the first EJC in a 1 Hz train, with a minimum intertrain interval of 1.5 minutes. Each data point represents the average of 2-3 EJCs per preparation, and no significant variation between EJCs recorded in different trains from the same preparation was observed. mEJC amplitude and frequency were determined from a continuous 40.875 second record of spontaneous activity in muscle 6 in each preparation.

### Pavlovian Olfactory Conditioning

Two-to-three-day-old adult flies were subjected to a Pavlovian conditioning procedure (Tully et at., Cell, 79:35-47 (1994); Tully and Quinn, J. Comp. Physiol., 157:263-277 (1985)). Briefly, groups of about 100 flies received one training session, during which they were exposed sequentially to one odor (CS+) paired with foot-shock and then a second odor (CS−) without foot-shock. For massed and spaced training, flies received ten training sessions as above. For massed training, one training session followed another with no rest interval in between. For spaced training, a 15 minute rest interval was introduced.

Conditioned odor avoidance was tested at various intervals after a single training session or one- and seven-day after massed or spaced training. During the test trial, flies were exposed simultaneously to the CS+ and CS− in a T-maze. After two minutes, flies were trapped in either T-maze arm, anesthetized and counted. From this distribution, a performance index (PI) was calculated, so that a 50:50 distribution (no memory) yielded a PI of zero and a 0:100 distribution away from the CS+ yielded a PI of 100.

### Sensorimotor Responses

The flies' “task-relevant” abilities to sense the odors or footshock (separately) and to escape from them were quantified as in Tully et al. (Cell, 79:35-47 (1994)). Briefly, to assess the flies' task-relevant ability to sense odors, two odors were passed through the T-maze arms as in the olfactory conditioning experiments. Naive flies were lowered to the center of the T-maze, and their odor avoidance at different odor concentrations was quantified as above (Boynton et al., Genetics, 131:655-672 (1992)). To assess the flies' task-relevant ability to sense footshock and to escape from it, “grid-tubes” were attached to each T-maze arm. Voltage (DC current) was applied to one arm of the T-maze but not to the other arm. Naive (untrained) flies were lowered to the center of the T-maze, and their electroshock avoidance was quantified as above (Luo et al., Neuron, 9:595-605 (1992)). Prior to behavioral investigations, the genetic backgrounds of mutant and transgenic flies were equilibrated to the appropriate wild-type strain by at least five generations of backcrossing. At least 10 virgin females, carrying one copy of the particular mw⁺ marker, were mated to at least 10 wild-type males in each of these backcross generations. In this manner, recombination around the mw⁺ P element insertion also occurs and serves to equilibrate even the linked chromosome.

### Data Analysis

Planimetrics (FIG. 5B) data were distributed normally. Results for mushroom bodies and central complex were analyzed. For mushroom bodies, the average volume of left and right hemispheres from four genotypes [wild-type (+), nal^{P1}, +/nal^{le60} and nal^{P1}/nal^{le60}] and two sexes (male and female) were subjected to two-way ANOVAs with SEX (F_{(1,72)}=0.006; P=0.984) and GENOtype (F_{(3,72)}=0.691; P=0.560) as main effects and SEX×GENO (F_{(3,72)}=0.052; P=0.984) as the interaction terms. For central complex, the combined volume of all four substructures from four genotypes [wild-type (+), nal^{P1}, +/nal^{le60} and nal^{P1}/nal^{le60}] and two sexes (male and female) were subjected to two-way ANOVAs with SEX (F_{(1,72)}=0.910; P=0.343) and GENOtype (F_{(3,72)}=3.283; P=0.026) as main effects and SEX×GENO (F_{(3,72)}=0.436; P=0.728) as the interaction terms. Six planned comparisons were judged significant if the experiment wise α′≦0.009.

Behavioral experiments were carried out in a balanced fashion with n=2 PIs per group per day (Tully et al., Cell, 79:35-47 (1994)). Replicate days were combined to generate final Ns. Untransformed data were analyzed parametrically with JMP (version 3.0) statistical software (SAS Institute Inc.). All pairwise comparisons from ANOVAs were planned, and the critical values for individual comparisons were adjusted to maintain an experimentwise error rate of alpha=0.05 (Sokal and Rohlf, Biometry, New York: Freeman (1997)). When explicitly stated in the text as “significant” or “not significant” (similar), the corresponding adjusted P values were P<0.05 or P>0.05.

Memory retention after one training session in wild-type and mutant flies (FIG. 1A): PIs from two strains (Can-S and na^{P1}) and six retention intervals were subjected to two-way ANOVA with STRAIN (F_{(1,60)}=43.3; P<0.001) and INTERVAL (F_{(5,60)}=19.1; P<0.001) as main effects and STRAIN×INTERVAL (F_{(5,60)}=0.3; P=0.892) as the interaction term. The significant effect of STRAIN and nonsignificant STRAIN×INTERVAL interaction term indicates that the mutant memory curve was reduced but decayed away with normal kinetics.

Memory retention immediately after one training session in wild-type flies, nal^{P1} mutants and homo-or hetero-allelic combinations of revertant excision alleles (FIG. 1C): PIs from nine STRAINS were subjected to a one-way ANOVA with STRAIN (F_{(8,45)}=13.0; P<0.001) as the main effect. Subsequent planned pairwise comparisons were adjusted for experimentwise error with Dunnet's method.

Memory retention immediately after one training session in wild-type flies, nal^{P1} mutants and heterozygous or heteroallelic combinations of lethal excision alleles (FIG. 1D): PIs from nine STRAINS were subjected to a one-way ANOVA with STRAIN (F_{(8,93)}=14.6; P<0.001) as the main effect. Subsequent planned pairwise comparisons were adjusted for experimentwise error with Dunnet's method.

Memory retention immediately after one training session in wild-type flies, nal^{P1} mutants and transgenic lines raised at 25° C. (FIG. 3B): PIs from six strains (+, nal^{P1}, hsp-Adf1⁺-8, hsp-Adf1⁺-32, hsp-Adf1⁺-11 and nal^{P1};hsp-Adf1⁺-11) were subjected to a one-way ANOVA with STRAIN (F_{(5,30)}=6.1; P<0.001) as the main effect. Subsequent planned pairwise comparisons were adjusted for experimentwise error with Dunnet's method.

Memory retention immediately after one training session in wild-type flies, nal^{P1} mutants and transgenic lines raised at 18° C. (FIG. 3C): PIs from six strains (+, nal^{P1}, hsp-Adf1⁺-8, hsp-Adf1⁺-32, hsp-Adf1⁺-11 and nal^{P1}; hsp-Adf1⁺-11) were subjected to a one-way ANOVA with STRAIN (F_{(5,42)}=10.6; P<0.001) as the main effect. Subsequent planned pairwise comparisons were adjusted for experimentwise error with Dunnet's method.

Memory retention immediately after one training session in wild-type flies, nal^{P1} mutants and transgenic lines raised at 18° C. and then shifted to 25° C. for three days as adults (FIG. 3D): PIs from six strains (+, nal^{P1}, hsp-Adf1⁺-8, hsp-Adf1⁺-32, hsp-Adf1⁺-11 and nal^{P1};hsp-Adf1⁺-11) were subjected to a one-way ANOVA with STRAIN (F_{(5,30)}=4.8; P=0.002) as the main effect. Subsequent planned pairwise comparisons were adjusted for experimentwise error with Dunnet's method.

Memory retention immediately after one training session in wild-type flies, nal^{P1} mutants and transgenic lines before and after acute heat shock three hours before training (FIG. 3E): PIs from four strains (+, nal^{P1}, nal^{P1}; hsp-Adf1⁺-8 and nal^{P1};hsp-Adf1⁺-11) and two heat shock conditions (−hs and +hs) were subjected to two-way ANOVA with STRAIN (F_{(3,40)}=29.1; P<0.001) and HEATSHOCK (F_{(1,40)}=13.3; P=0.001) as main effects and STRAIN×HEATSHOCK (F_{(3,40)}=0.9; P=0.433) as the interaction term. The four subsequent planned comparisons were judged significant if P<0.013.

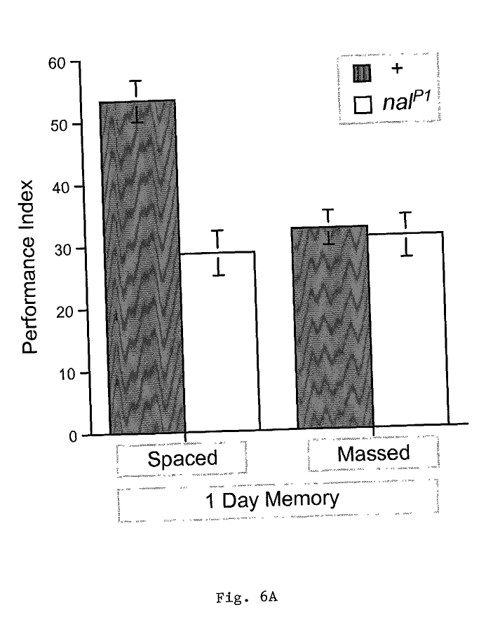

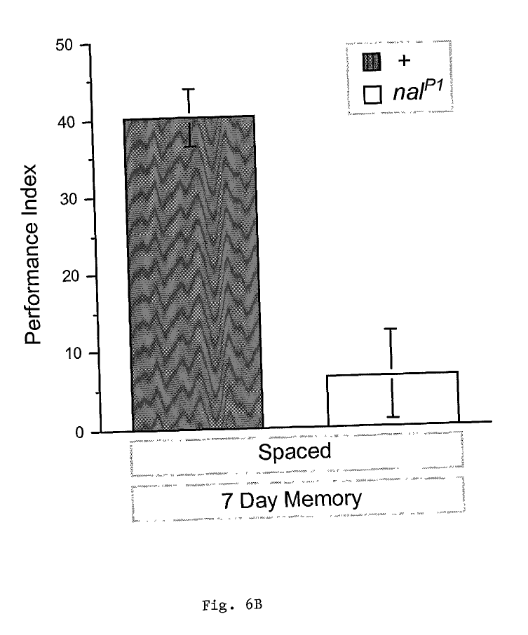

One day memory after spaced or massed training in wild-type flies and nal^{P1} mutants (FIG. 6A): PIs from two strains (+ and nal^{P1}) and two training protocols (spaced and massed) were subjected to a two-way ANOVA with STRAIN (F_{(1,60)}=15.767; P<0.001) and TRAINing (F_{(1,60)}=8.471; P=0.005) as main effects and STRAIN×TRAIN (F_{(1,60)}=12.308; P=0.001) as the interaction term. The four subsequent planned comparisons were judged significant if P<0.013.

Seven day memory after spaced or massed training in wild-type flies and nal^{P1} mutants (FIG. 6B): PIs from two strains (+ and nal^{P1}) were subjected to a one-way ANOVA with STRAIN (F_{(1,10)}=24.4; P=0.001) as the main effect.

Shock Reactivity in wild-type flies and nal^{P1} mutants (Table): PIs from two strains (+ and nal^{P1}) and six voltages (0, 10, 20, 30, 40 and 60) were subjected to a two-way ANOVA with STRAIN (F_{(1,84)}=0.3; P=0.596) and VOLT (F_{(5,84)}=39.3; P<0.001) as main effects and STRAIN×VOLT (F_{(5,84)}=0.2; P=0.968 as the interaction term. No significant effects of STRAIN or STRAIN×VOLT were detected.

Olfactory Acuity in wild-type flies and nal^{P1} mutants (Table): PIs from two strains (+ and nal^{P1}) and two odor concentrations (10⁰ and 10⁻²) were subjected to two-way ANOVAs for OCT and MCH separately. For OCT, STRAIN (F_{(1,20)}=0.1; P=0.772) and CONC (F_{(1,20)}=11.8; P<0.001) were main effects, and STRAIN×CONC (F_{(1,20)}=0.7; P=0.42) was the interaction term. For MCH, STRAIN (F_{(1,20)}=0.2; P=0.668) and CONC (F_{(1,20)}=73.5; P<0.001) were the main STRAIN×CONC (F_{(1,10)}=0.1; P=0.749) was the interaction term. For each odor, no significant effects of CONC or STRAIN×CONC were detected.

### Example 1

### Isolation of the Pavlovian Memory Mutant nalyot

In a behavioral screen for olfactory memory mutants, 2,182 transposant strains were each assayed for three-hour memory retention after a single training session of a Pavlovian olfactory conditioning procedure (cf. Tully and Quinn, J. Comp. Physiol., 157:263-277 (1985)). Four mutant lines displayed lower than normal memory scores and normal perception of the component stimuli: latheo (Boynton and Tully, Genetics, 131:655-672 (1992); Pinto et al., Neuron, 23:45-54 (1999); Rohrbough et al., Neuron, 23:55-70 (1999)), linotte (Dura et al., J. Neurogenet., 9:1-14 (1993); Bolwig et al., Neuron, 15:829-842 (1995); Simon et al., Mech. Dev., 76:42-55 (1998)), golovan, and nalyot (nal).

In wild-type flies, a single session of Pavlovian olfactory conditioning produces memory that decays to baseline levels by 24 hours, is insensitive to cycloheximide feeding, and is not disrupted by induced expression of a CREB-repressor transgene (Tully et al., Cell, 79:35-47 (1994); Yin et al., Cell, 79:49-58 (1994)). Immediately after such training, nal mutants showed a mild but significant disruption of conditioned odor avoidance behavior (FIG. 1A). Thereafter, memory decay (the slope of the forgetting curve) was normal in nal mutants, suggesting a defect in learning (acquisition) or very early (immeasurable) memory processing.

Despite their performance defect in Pavlovian memory, nal mutants displayed normal “task-relevant” sensorimotor responses. Results are shown in the Table.

TABLE
Olfactory Acuity and Shock Reactivity Are Normal in nal^{P1} Mutants.

| | Olfactory Acuity^{a} | | | | | | |
|---|---|---|---|---|---|---|---|
| | OCT Dilution | | MCH Dilution | | Shock Reactivity^{b} | | |
| Strain | 10⁰ | 10⁻² | 10⁰ | 10⁻² | 60V | 40V | 20V |
| +(CanS) | 58 ± 6 | 43 ± 6 | 70 ± 5 | 18 ± 9 | 85 ± 4 | 71 ± 5 | 32 ± |
| | | | | | | | 6 |
| nal^{P1} | 65 ± 4 | 40 ± 5 | 71 ± 3 | 22 ± 5 | 86 ± 4 | 73 ± 5 | 37 ± |
| | | | | | | | 13 |

^{a}For OA: n = 8 PIs per group, except n = 4 for Can-S and nal^{P1} at OCT (1:100).
^{b}For SA: n = 8 PIs per group.

Wild-type and mutant flies showed similar levels of shock reactivity at the intensity (60V) used for Pavlovian training and at lower ones. Likewise, wild-type and mutant flies exhibited similar levels of olfactory acuity to MCH and OCT at the intensity used for Pavlovian training and with a 100-fold dilution. Thus, the performance defect of nal mutants after Pavlovian training cannot be explained by disruptions in the perception of, or responses to, the stimuli presented. Rather, nal mutants appear unable to associate the two stimuli normally when they are presented together.

### Example 2

### The nal Memory Defect is Caused by a Transposon Insertion

The nal^{P1} mutation is associated with a single P element (PlacW) insertion at cytological position 42D1-2, located on the proximal right arm of the second chromosome. PlacW encodes a readily scoreable marker, the mini-white eye color gene. This marker was selected against in a standard mating scheme to remobilize the P element (cf. Dura et al., J. Neurogenet., 9:1-14 (1993)) and generated 50 independent excision strains (FIG. 1B).

The excision strains were screened for precise excisions (revertants) to confirm that the nal memory defect was associated with the original P element insertion. By performing PCR on genomic DNA with primers flanking the P insertion site, ten candidate precise excisions were identified. All were homozygous viable. Three of these strains were arbitrarily selected for further analysis by PCR cloning and sequencing; all were molecular revertants. These strains, nal^{ve25A}, nal^{ve80} and nal^{ve96}, then were tested for memory individually and in heteroallelic combinations with each other. All six genotypes yielded normal memory (FIG. 1C), indicating that the nal^{P1} P insertion was responsible for the nal memory defect.

Of the 50 excision strains, nearly one-third were homozygous lethal, suggesting that the nal P element was linked tightly to an essential locus. Strains with deletions of the P element that extended into the flanking genomic sequences were homozygous lethal and included nal^{le55} and nal^{le60} (FIG. 1B). These strains complemented the lethal deficiencies Df(2R) cn88b and Df(2R) cn87e, but not Df(2R)42 and Df(2R)nap12. Genomic mapping of nal^{le55} revealed a deletion extending at least 13 kb to the right of the P element. Mapping of nal^{le60} revealed two molecular perturbations: (1) a closely linked 1.8 kb deletion in the flanking region and (2) 30 bp of terminal P element sequences along with an 8 bp target site duplication (FIG. 1B). Mapping of viable excision nal^{ve48} revealed the same 38 bp disruption as that in nal^{le60} with no discernible disruptions of flanking genomic regions.

Memory was examined in behavioral complementation tests in strains carrying different combinations of the nal^{P1}, nal^{le60}, nal^{ve48} and nal⁺ alleles (FIG. 1D). All three alleles are recessive to the wild-type allele for memory. In heteroallelic combinations, only nal^{le60}/nal^{P1} flies show a memory deficit. Normal memory for nal^{ve48}/nal^{P1} flies indicated that the failure of nal^{le60} to complement nal^{P1} must arise from its 1.8-kb deletion rather than the 38 bp duplication. These results verify that the nal^{P1} P element insertion disrupts olfactory memory.

### Example 3

### The nal Mutation is a P Insertion in the Adf1 Transcription Unit

A 9.4 kb Sac II fragment flanking the nal P element was isolated by plasmid rescue. The corresponding genomic region was cloned, and then two genes in the vicinity of the nal P element were mapped (FIG. 2A). The results revealed two independent transcription units, oriented head to head and separated by a maximum distance of 240 bp. Transcribing to the left is cn20, a previously uncharacterized gene, which encodes an unspliced 1.3 kb RNA product. Sequencing of a corresponding 1,263 bp cDNA predicts a 395 amino acid open reading frame with homology to stress proteins. Transcribing to the right is Adf1. The ADF1 protein was identified by its specific binding to the distal promoter of the Alcohol dehydrogenase (Adh) gene (Heberlein et al., Cell, 41:965-977 (1985)) and was then purified and cloned (England et al., J. Biol. Chem., 265:5086-5094 (1990); England et al., Proc. Natl. Acad. Sci. USA, 89:683-687 (1992)). Mapping of more than 10 Adf1 cDNAs onto the genomic region revealed a single 3′ end and three introns (FIG. 2A). The distal two exons appear to be constitutively spliced from all transcripts. In contrast, a 114 bp intron, located upstream of coding region, is sometimes retained in the mature transcript. This 5′ heterogeneity potentially can give rise to two different amino-terminal sequences (FIG. 2A). The mRNA retaining the intron encodes a 253 amino acid protein, whereas removal of the intron introduces a new methionine (i2) in frame with the old one (i1), resulting in the potential addition of a nine amino acid stretch, MHTLTAAIG (SEQ ID NO:10). The significance of this potential heterogeneity is unclear. Engineered removal of 12 amino acids from the amino terminus of the smaller form abolishes DNA binding and transactivation activities in Schneider cell transfections (Cutler et al., Mol. Cell. Biol., 18:2252-2261 (1998)); the longer version described herein appears to have normal DNA binding and transactivation activities.

The nal^{P1} P element is positioned within the large intron of the Adf1 transcription unit, 147 bp downstream of the splice donor site (FIG. 2A). This intron disrupts the Adf1 coding region at the predicted junction of two α-helices, which constitute its signature myb-related helix-turn-helix motif. Construction of the genomic and transcript map also revealed that the nal^{le60} lesion extends from 351 bp upstream of the terminal exon to 150 bp past the poly(A) site (FIG. 2A). The nal^{le60} allele is therefore a null mutation, removing most of the protein (210 amino acids), including essential domains required for DNA binding and transactivation. Given that the lethality of nal^{le60} co-maps to the region, Adf1 is an essential gene. Moreover, the failure of nal^{P1} to complement nal^{le60} for memory provides strong genetic evidence that disruptions of Adf1 can affect adult olfactory memory.

### Example 4

### nal^{P1} is a Hypomorphic Allele of Adf1.

Consistent with our genetic analyses, several lines of molecular evidence indicate that nal is a mutation of Adf1. The first derives from Northern blot analyses of Adf1 and cn20 poly(A) RNA in adult heads and bodies (FIG. 2B). Relative to levels of control RNA (rp49), cn20 RNA levels are similar in nal^{P1} mutants and wild-type flies, whereas Adf1 RNA levels from mutants are reduced approximately two-fold in both fractions. These differences were quantified by Phosphorimager analysis (for heads: 1633±49 (wild-type) vs. 775±51 (nal^{P1}); n=12 (triple determination of 4 independent head extracts)).

Overexposures of RNA blots failed to show any extra bands in the nal^{P1} lanes, indicating that the large (˜11 kb) P element insertion was not grossly disrupting mRNA processing (cf. Pinto et al., Neuron, 23:45-54 (1999) for latheo). For higher resolution, RT-PCR was used to evaluate both the fidelity and relative use of processing signals in mutant heads (and bodies). All Adf1 introns in nal^{P1} mutants were excised normally, with no evidence of cryptic splice site use. These results confirm those from Northern analyses: nal^{P1} mutants have a general decrease in Adf1 RNA levels with normal splicing fidelity.

Results from Western blot analyses corroborate these findings (FIG. 2C). Consistent with the observations of England et al. (Proc. Natl. Acad. Sci. USA, 89:683-687 (1992)), a single protein species of 34 kD was observed in both mutant and wild-type heads. Relative to the levels of two control proteins, the TATA binding transcription factor (TBP) and the microtubule protein α-tubulin (TUB), ADF1 protein levels are reduced in nal heads by at least two-fold. A similar result was observed in the bodies of nal^{P1} animals but expression levels were too low to quantify. Together, these molecular studies suggest that the nal^{P1} mutant is a hypomorph, showing a decrease in both Adf1 RNA and protein levels, with no aberrant splicing or translation product. This interpretation is fully consistent with the genetic complementation analyses presented in FIG. 1D, as well as with immunocytochemical and transgenic rescue experiments described below.

### Example 5

### Adult Expression of an hsp-Adf1 Transgene Can Rescue the nal^{P1} Memory Defect

As Adf1 is expressed in both the developing and adult animal (England et al., Proc. Natl. Acad. Sci. USA, 89:683-687 (1992)), inducible transgene technology was employed to address its function in adult behavior. Briefly, a 1,603 bp cDNA encoding the entire ADF1 protein was inserted downstream of the hsp70 promoter in the transformation vector pCaSpeR-hs. Three independent transgenic strains were generated using standard microinjection technology, each expressing the wild-type Adf1 gene under control of the hsp70 promoter. In addition to conferring acute inducibility, the hsp promoter drives constitutive (“leaky”) transgene expression.

Given the essential nature of Adf1, “leaky” expression of the Adf1⁺ transgenes was first manipulated by raising the animals at different temperatures. Northern blot analysis of adult head RNA revealed leaky Adf1 transgene expression in nal^{P1};hsp-Adf1⁺-11 adults raised continuously at either 18° C. or 25° C. (FIG. 3A, lanes 11,12). Leaky expression of hsp-Adf1⁺-11 at 18° C. or 25° C. is sufficient to rescue the lethality of nal^{le60}/nal^{le55} null mutants. These results confirmed the essential role of Adf1 for viability and set the stage for behavioral studies.

To determine whether induced transgene expression could rescue the memory defect of nal^{P1} mutants, nal^{P1};hsp-Adf1⁺-11 adults were raised continuously at 18° C. nal^{P1};hsp-Adf1⁺-11 animals raised continuously at 18° C. showed partial behavioral rescue (FIG. 3C). The same animals showed complete rescue when raised at 18° C. during development and then shifted to 25° C. for the first three days after eclosion (FIG. 3D). These results suggest that adult expression of Adf1 is required for normal memory.

### Example 6

### Proper Developmental Expression of Adf1 is Required for Optimal Adult Memory

Adf1 expression also has a developmental role. Two lines of investigations supported this notion. First, temperature-shift experiments of hsp-Adf1⁺ reveal a deleterious period of overexpression that appears confined to development. When raised at 25° C., olfactory memory in +; hsp-Adf1⁺-8, -11, and -32 lines, (and in nal^{P1};hsp-Adf1⁺-11 flies) was lower-than-normal (FIG. 3B). When raised at 18° C., however, memory in each line was similar to that in wild-type flies (FIG. 3C), and was not diminished when the 18° C. rearing period was followed by a shift to 25° C. for three days as adults (FIG. 3D).

Second, acute manipulations of ADF1 expression in adults fail to produce a specific impairment of memory formation. Memory is normal in adult hsp-Adf1⁺-8; +animals, when they are raised at 18° C. to minimize leaky ADF1 expression and then given two 30 minutes heat-shocks (37° C.) 24 and 3 hours prior to training (81±3 for wild-type versus 78±2 for +; hsp-Adf1⁺-8; n=6 PIs per genotype). Similarly, a 30 minute heat shock (37° C.) in nal^{P1};hsp-Adf1⁺-11 or hsp-Adf1⁺-8; nal^{P1} adults three hours before training neither rescues nor worsens their early memory deficits (cf. Bolwig et al., Neuron, 15:829-842 (1995); Grotewiel et al., Nature, 391:455-460 (1998)), despite a dramatic induction of the ADF-1 protein (FIG. 3E). Together, these results suggest that regulated Adf1 expression during development is critical for optimal adult memory.

### Example 7

### Overexpression of Adf1 in the Nervous System Can Be Lethal

To investigate the general role of Adf1 during nervous system development, three independent transgenic strains expressing Adf1⁺ under control of the UAS promoter were generated (UAS-Adf1⁺-201B, UAS-Adf1⁺-202 and UAS-Adf1⁺-203). Each strain was crossed to a panel of GAL4 enhancer trap lines with various patterns of expression in the nervous system: elav-GAL4 and scabrous-GAL4 express widely in the CNS (Kidd et al., Neuron, 1:25-33 (1998)). MZ1580 and C321c express primarily in developing glia (Hidalgo et al., Development, 121:3703-3712 (1995)). C747, 201Y, 238Y and OK107 express preferentially in mushroom bodies (Connolly et al., Science, 274:2104-2107 (1996)). Within the central complex, an adult brain structure, OK348 expresses preferentially in the fan-shaped body; and C232, the most specific line of all, expresses with near exclusivity in the ellipsoid body (Connolly et al., Science, 274:2104-2107 (1996)). When the UAS-Adf1⁺ transgenes were expressed with these GAL4 lines, all failed to survive to adulthood, except for C232/+;UAS-adf1⁺/+flies. The onset of lethality varied from late embryo to late pupa.

Together, these observations suggest that the levels of ADF1 must be maintained within a narrow range of expression during development to ensure proper biological function. Such findings are consistent with our behavioral investigations, which also revealed a tight dependence of optimal memory upon proper Adf1 expression.

### Example 8

### ADF1 Shows Widespread Expression in the Embryonic, Larval, and Adult Nervous Systems

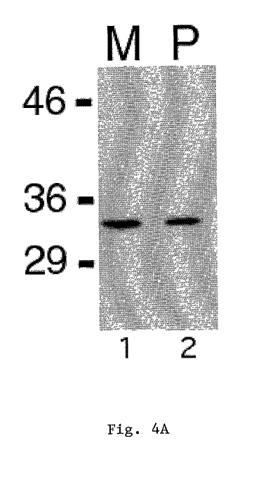

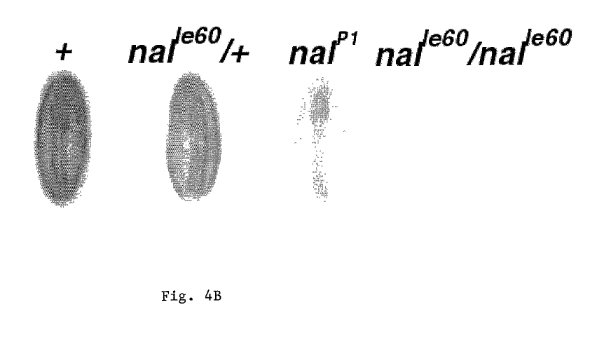

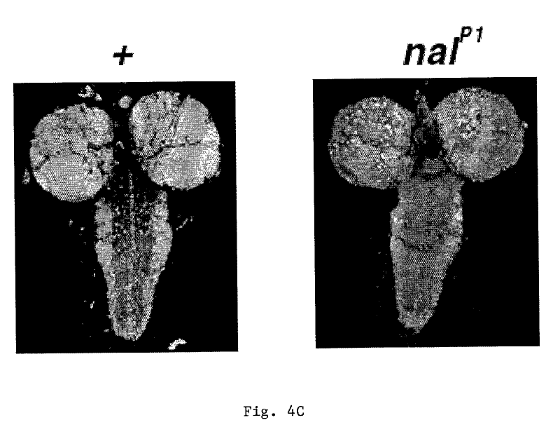

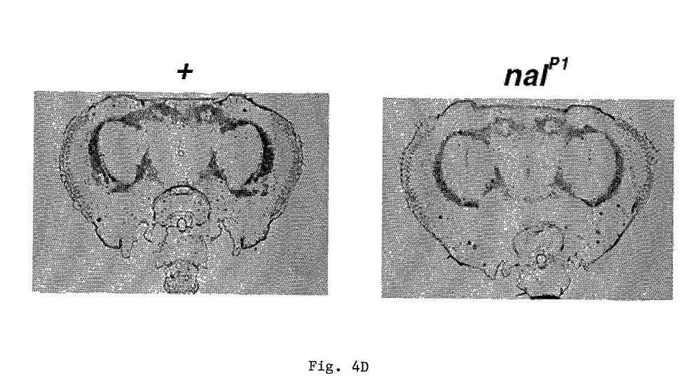

A monoclonal antibody (MAb Adf1-17) against ADF1 was produced, which recognizes a single band of approximately 34 kD on Western blots of head tissue (FIG. 4A; cf. England et al., Proc. Natl. Acad. Sci. USA, 89:683-687 (1992)). No ADF1 expression was detected in embryos 45-minutes after egg laying, which is before the onset of zygotic transcription. Widespread expression was seen, however, from approximately two hours after egg laying onwards. These observations support the notion that ADF1 is expressed only in zygotes (cf. England et al., Proc. Natl. Acad. Sci. USA, 89:683-687 (1992)). At stage 16, wild-type embryos show widespread nuclear expression, with intense staining in the ventral nerve cord (FIG. 4B). Homozygous nal^{le60}/nal^{le60} embryos, on the other hand, show no specific staining, confirming their predicted lack of ADF1 expression and the specificity of Mab Adf1-17 immunoreactivity in situ. Notably, nal^{P1} homozygotes show significantly reduced levels of ADF1, compared even to nal^{le60}/+heterozygous flies carrying only one copy of Adf1⁺ (FIG. 4B).

In third instar larva, ADF1 expression is widespread in the nervous system. In homozygous nal^{P1} mutants, ADF1 levels appear uniformly reduced in the central brain and ventral ganglia (FIG. 4C).

In adult heads, ADF1 expression is observed only in nuclei throughout the adult brain and with no apparent preferential expression (FIG. 4D). Here again, ADF1 immunoreactivity is uniformly reduced in homozygous nal^{P1}/nal^{P1} mutants. Similar results were obtained with use of a second monoclonal line, MAb Adf1-8. Thus, at all developmental stages examined, ADF1 expression appears widespread, quantitatively reduced in mutants, and restricted to the nuclear compartment.

### Example 9

### Genetic Disruptions of Adf1 Do Not Yield Gross Morphological Defects in Embryonic or Adult Nervous Systems

Given the inviability of homozygous null mutants, their lethal phase was examined in more detail. About 75% of nal^{le60}/nal^{le60} mutants die as mature embryos. The remaining 25% hatch and manifest basic behavioral responses, such as forward and backward locomotion to tactile stimuli. These animals are sluggish, however, fail to grow normally and die before the third instar stage. Mutant nal^{le55}/nal^{le55} and nal^{le55}/nal^{le60} flies show a developmental etiology similar to that of nal^{le60}/nal^{le60} animals. These results suggest an onset of lethality beginning late in embryogenesis and, in some cases, extending into early stages of larval development.

In late stage homozygous nal^{le60}/nal^{le60} embryos, development of the CNS, PNS, and trachea appeared normal, as did expression of the cell adhesion molecule FAS II (expressed in a subset of PNS and CNS axons), REPO (a nuclear protein expressed in proliferating glia), and the patterning genes EVE and EN (which function late in embryogenesis to guide differentiation of a number of cell-types, including a subset of neurons). These data suggest unperturbed neuroanatomy during embryogenesis.

Two hypomorphic genotypes, nal^{P1} homozygotes and nal^{P1}/nal^{le60} mutants, routinely survive to adulthood, thereby permitting an evaluation of gross anatomical structures in the adult brain. The mushroom bodies and central complex, in particular, have been implicated in associative olfactory learning (deBelle et al., Science, 263:692-695 (1994); Connolly et al., Science, 274:2104-2107 (1996)). Visual inspection of frontal sections from wild-type and mutant adult brains revealed no obvious malformations in gross anatomy. For example, the α and β lobes of the mushroom bodies showed normal fasciculation and orientation. Likewise, sub-structures of the central complex appeared intact; the fan-shaped body was not split and there was no ventral opening or flattening of the ellipsoid body. A more quantitative “planimetric” assessment of neuropillar volumes of adult mushroom body calyces and central complex analysis in wild-type (+), heterozygous (+/nal^{le60}) flies and hypomorphic mutants (nal^{P1}/nal^{le60}) was also carried out. No significant differences in males or females for either anatomical region were detected.

### Example 10

### Adf1 Affects the Number of Synaptic Boutons at the Larval Neuromuscular Junction

The failure of morphological studies to reveal gross defects in the nervous system led us to examine Adf1 function at the larval neuromuscular junction (NMJ). At this peripheral synapse, structure can be examined by analyzing the numbers and distributions of variocosities (synaptic boutons), which are the sites of neurotransmitter release from motor neurons onto muscles, and synaptic function can be analyzed by recording spontaneous and evoked transmitter release onto muscle. Since Adf1 expression is widespread in the larval ventral ganglia and reduced in nal^{P1} mutants (FIG. 3B), the NMJ was considered a likely place to quantify the role of Adf1 in the development of synaptic connections.

Synaptic boutons on muscles 6 and 7, which are innervated by the same motor neurons, were analyzed and a striking correlation between ADF1 expression levels and bouton number was discovered. When ADF1 expression is reduced (in nal^{P1} mutants), the number of boutons is significantly reduced (FIGS. 5A-5B). Conversely, when ADF1 expression is greater-than-normal (in hsp-Adf1⁺-8 or -11 animals raised at temperatures that produce leaky expression of the transgene) the number of boutons is significantly increased (FIGS. 5A-5B). Opposing changes in ADF1 expression levels, therefore, are correlated with opposing effects on synaptic structure at the NMJ, yielding a difference between these extremes of 30%.

Three lines of evidence support the specificity of these observations. First, the change of synapse number in nal^{P1} mutants does not appear to arise from a defect in neuronal proliferation. Two motor neurons normally innervate muscle 6, terminating in type 1s and type 1b boutons. Mutant nal^{P1} and hsp-Adf1⁺-8 animals show both types of boutons as well as normal levels and distributions of synaptotagmin within them (FIG. 5A). Second, the Adf1 null allele, nal^{le60}, fails to complement the bouton defect of nal^{P1} mutants—just as it fails to complement the memory defect (FIG. 1D). Third, leaky expression of an hsp-Adf1⁺ transgene is capable of rescuing the bouton defect of nal^{P1};hsp-Adf1⁺-11 mutants (FIG. 5B), just as it was capable of rescuing the memory defect (FIG. 1D). Collectively, these observations suggest that ADF1 plays a role in post-mitotic stages of neuronal development. Given the reciprocal actions of ADF1 on bouton number, this role appears to be at the level of synapse formation and/or maturation.

### Example 11

### Adf1 Minimally Affects Basal Synaptic Transmission at the Larval Neuromuscular Junction

Spontaneous and evoked synaptic transmission were evaluated in wild-type larvae, nal^{P1} mutants and hsp-Adf1⁺-8 or hsp-Adf1⁺-11 transgenic animals. EJC and mEJC amplitudes did not differ among the different genotypes, being approximately 135 and 0.72 nA, respectively (FIGS. 5C and 5D). Thus, quantal content, the number of synaptic vesicles released per action potential, is unaffected by these perturbations of ADF1 levels (FIG. 5D).

mEJC frequency also was normal in nal mutant and hsp-Adf1⁺ transgenic flies. However, a marked increase was detected in mEJC frequency for hsp-Adf1⁺-8 transgenic flies (FIGS. 5B and 5D). This observation may be spurious, however, since it did not appear in hsp-Adf1⁺-11 flies when they were raised at 21° C. (FIG. 5A) or at 25° C. (to increase leaky over-expression levels). Hence, the most likely explanation is a strain-specific effect unrelated to Adf1 function, produced either by the (transgenic) P element insertion site or by inadvertent genetic background differences.

The investigations described herein have focused on basal transmission. More strenuous protocols, such as repetitive stimulation, can be used to unmask physiological defects. Preliminary observations suggest that such effects, if present, are subtle. Together, these data support a preferential role for Adf1 in maturation of synaptic structure rather than function.

### Example 12

### The nal Mutation Abolishes Long-Term Memory Formation

ADF1's widespread expression in the adult brain and its involvement in synapse formation at the NMJ suggested that disruption of this transcription factor might yield a defect in adult long-term memory formation. In essence, long-term memory likely requires increases in both synaptic structure and function. If nal blocked an experience-dependent increase in synaptic structure, then, by analogy to observations at the NMJ (cf. Davis et al., Neuron, 17:669-679 (1996)), it should block any concomitant increase in synaptic function.

In wild-type flies, ten sessions of Pavlovian olfactory conditioning produce two types of long-lasting memory that can be distinguished on the basis of behavioral, genetic, and pharmacological properties (Tully et al., Cell, 79:35-47 (1994)). Ten “massed” training sessions (with no rest interval between sessions) gives rise only to an anesthesia-resistant memory (ARM). ARM decays to baseline within four days after training, is immune to inhibitors of protein synthesis, is not disrupted by over-expression of a dominant-negative CREB transgene (Yin et al., Cell, 79:49-58 (1994)), and is disrupted in radish mutants (Tully et al., Cell., 79:35-47 (1994)). Ten “spaced” training sessions (with a 15 minute rest interval between sessions) gives rise to ARM and a bona fide long-term memory (LTM). LTM persists for at least seven days, is sensitive to inhibitors of protein synthesis, is disrupted by over-expression of a dominant-negative CREB transgene, is normal in radish mutants, and is induced after only one training session by over-expression of a CREB activator transgene (Tully et al., Cell, 79:35-47 (1994); Yin et al., Cell, 79:49-58 (1994); Yin et al., Cell, 81:107-115 (1995)). Thus, LTM appears to be transcription-dependent, while ARM does not.

One-day memory retention was quantified in wild-type flies and nal^{P1} mutants after they were subjected to spaced or massed training in experimenter-blind, balanced experiments replicated over six days. At this retention interval, memory after spaced training in wild-type flies (LTM+ARM) is roughly twice that of memory after massed training (ARM only; FIG. 6A). In nal^{P1} mutants, one-day memory after massed training was similar to that of wild-type flies, suggesting that ARM is normal in mutant flies. In contrast, one-day memory after spaced training in mutant flies was significantly lower than that in wild-type flies and, in fact, was similar to one-day memory after massed training in wild-type (and mutant) flies. These observations suggest that LTM is absent in nal^{P1} mutants.

To confirm this notion, seven-day memory also was assayed. Significant levels of performance are seen at this retention interval only after spaced training and only with the formation of protein synthesis- and CREB-dependent LTM (Tully et al., Cell, 79:35-47 (1994); Yin et al., Cell, 79:49-58 (1994)). No significant seven-day memory was detected in nal^{P1} flies (FIG. 6B). Together, these behavioral experiments indicate that LTM is abolished in nal mutants.

The teachings of all the articles, patents and patent applications cited herein are incorporated by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating an animal with a defect in long term memory formation associated with a defect in an ADF1-like molecule comprising treating said animal to increase expression of an Adf1-like gene relative to expression of said Adf1-like gene in said animal prior to said treatment.

2. The method of claim 1 wherein said animal is a mammal.

3. The method of claim 1 wherein said animal is a human.

4. The method of claim 1 wherein said animal is a Drosophila species.

5. A method of treating an animal with a defect in long term memory formation associated with a defect in an ADF1-like molecule comprising treating said animal to increase functional ADF1-like activity relative to the functional ADF1-like activity in said animal prior to said treatment.

6. The method of claim 5 wherein said animal is a mammal.

7. The method of claim 5 wherein said animal is a human.

8. The method of claim 5 wherein said animal is a Drosophila species.

9. A method of treating an animal with a defect in long term memory formation associated with a defect in an ADF1-like molecule comprising administering to said animal an effective amount of ADF1-like molecule, ADF1 analog, biologically active ADF1-like fragment or ADF1-like fusion protein.

10. The method of claim 9 wherein said animal is a mammal.

11. The method of claim 9 wherein said animal is a human.

12. The method of claim 9 wherein said animal is a Drosophila species.

13. A method of treating an animal with a defect in long term memory formation associated with a defect in an ADF1-like molecule comprising administering to said animal an effective amount of a nucleic acid sequence encoding ADF1-like molecule, ADF1 analog, biologically active ADF1-like fragment or ADF1-like fusion protein.

14. The method of claim 13 wherein said animal is a mammal.

15. The method of claim 13 wherein said animal is a human.

16. The method of claim 13 wherein said animal is a Drosophila species.

17. The method of claim 13 wherein the nucleic acid sequence is incorporated into a viral vector.

18. A method of treating an animal with a defect in long term memory formation associated with a defect in an ADF1-like molecule comprising administering to said animal a pharmaceutical agent which is capable of increasing expression of an Adf1-like gene in said animal, in an amount effective to increase expression of Adf1-like gene relative to expression of said Adf1-like gene in said animal prior to administration of said pharmaceutical agent, thereby resulting in treatment of said animal.

19. The method of claim 18 wherein said animal is a mammal.

20. The method of claim 18 wherein said animal is a human.

21. The method of claim 18 wherein said animal is a Drosophila species.

22. A method of treating an animal with a defect in long term memory formation associated with a defect in an ADF1-like molecule comprising administering to said animal a pharmaceutical agent which is capable of increasing functional ADF1-like activity in said animal, in an amount effective to increase the functional ADF1-like activity relative to the functional ADF1-like activity in said animal prior to administration of said pharmaceutical agent, thereby resulting in treatment of said animal.

23. The method of claim 22 wherein said animal is a mammal.

24. The method of claim 22 wherein said animal is a human.

25. The method of claim 22 wherein said animal is a Drosophila species.

26. A method of modulating long term memory formation in an animal comprising treating said animal to modulate expression of an Adf1-like gene.

27. The method of claim 26 wherein said animal is a mammal.

28. The method of claim 26 wherein said animal is a human.

29. The method of claim 26 wherein said animal is a Drosophila species.

30. A method of modulating long term memory formation in an animal comprising treating said animal to modulate functional ADF1-like activity.

31. The method of claim 30 wherein said animal is a mammal.

32. The method of claim 30 wherein said animal is a human.

33. The method of claim 30 wherein said animal is a Drosophila species.

34. A method of modulating long term memory formation in an animal comprising administering to said animal a pharmaceutical agent which is capable of modulating expression of an Adf1-like gene in said animal, in an amount effective to modulate expression of Adf1-like gene in said animal, thereby resulting in modulation of long term memory formation in said animal.

35. The method of claim 34 wherein said animal is a mammal.

36. The method of claim 34 wherein said animal is a human.

37. The method of claim 34 wherein said animal is a Drosophila species.

38. A method of modulating long term memory formation in an animal comprising administering to said animal a pharmaceutical agent which is capable of modulating functional ADF1-like activity in said animal, in an amount effective to modulate the functional ADF1-like activity in said animal, thereby resulting in modulation of long term memory formation in said animal.

39. The method of claim 38 wherein said animal is a mammal.

40. The method of claim 38 wherein said animal is a human.

41. The method of claim 38 wherein said animal is a Drosophila species.

42. A method of enhancing long term memory formation in an animal comprising treating said animal to increase expression of an Adf1-like gene relative to expression of said Adf1-like gene in said animal prior to said treatment.

43. The method of claim 42 wherein said animal is a mammal.

44. The method of claim 42 wherein said animal is a human.

45. The method of claim 42 wherein said animal is a Drosophila species.

46. A method of enhancing long term memory formation in an animal comprising treating said animal to increase functional ADF1-like activity relative to the functional ADF1-like activity in said animal prior to said treatment.

47. The method of claim 46 wherein said animal is a mammal.

48. The method of claim 46 wherein said animal is a human.

49. The method of claim 46 wherein said animal is a Drosophila species.

50. A method of enhancing long term memory formation in an animal comprising administering to said animal an effective amount of ADF1-like molecule, ADF1 analog, biologically active ADF1-like fragment or ADF1-like fusion protein.

51. The method of claim 50 wherein said animal is a mammal.

52. The method of claim 50 wherein said animal is a human.

53. The method of claim 50 wherein said animal is a Drosophila species.

54. A method of enhancing long term memory formation in an animal comprising administering to said animal an effective amount of a nucleic acid sequence encoding ADF1-like molecule, ADF1 analog, biologically active ADF1-like fragment or ADF1-like fusion protein.

55. The method of claim 54 wherein said animal is a mammal.

56. The method of claim 54 wherein said animal is a human.

57. The method of claim 54 wherein said animal is a Drosophila species.

58. The method of claim 54 wherein the nucleic acid sequence is incorporated into a viral vector.

59. A method of enhancing long term memory formation in an animal comprising administering to said animal a pharmaceutical agent which is capable of increasing expression of an Adf1-like gene in said animal, in an amount effective to increase expression of Adf1-like gene relative to expression of said Adf1-like gene in said animal prior to administration of said pharmaceutical agent, thereby resulting in enhancement of long term memory formation in said animal.

60. The method of claim 59 wherein said animal is a mammal.

61. The method of claim 59 wherein said animal is a human.

62. The method of claim 59 wherein said animal is a Drosophila species.

63. A method of enhancing long term memory formation in an animal comprising administering to said animal a pharmaceutical agent which is capable of increasing functional ADF1-like activity in said animal, in an amount effective to increase the functional ADF1-like activity relative to the functional ADF1-like activity in said animal prior to administration of said pharmaceutical agent, thereby resulting in enhancement of long term memory formation in said animal.

64. The method of claim 63 wherein said animal is a mammal.

65. The method of claim 63 wherein said animal is a human.

66. The method of claim 63 wherein said animal is a Drosophila species.

67. A method of treating an animal with a defect in synaptic plasticity associated with a defect in an ADF1-like molecule comprising treating said animal to increase expression of an Adf1-like gene relative to expression of said Adf1-like gene in said animal prior to said treatment.

68. The method of claim 67 wherein said animal is a mammal.

69. The method of claim 67 wherein said animal is a human.

70. The method of claim 67 wherein said animal is a Drosophila species.

71. A method of treating an animal with a defect in synaptic plasticity associated with a defect in an ADF1-like molecule comprising treating said animal to increase functional ADF1-like activity relative to the functional ADF1-like activity in said animal prior to said treatment.

72. The method of claim 71 wherein said animal is a mammal.

73. The method of claim 71 wherein said animal is a human.

74. The method of claim 71 wherein said animal is a Drosophila species.

75. A method of treating an animal with a defect in synaptic plasticity associated with a defect in an ADF1-like molecule comprising administering to said animal an effective amount of ADF1-like molecule, ADF1 analog, biologically active ADF1-like fragment or ADF1-like fusion protein.

76. The method of claim 75 wherein said animal is a mammal.

77. The method of claim 75 wherein said animal is a human.

78. The method of claim 75 wherein said animal is a Drosophila species.

79. A method of treating an animal with a defect in synaptic plasticity associated with a defect in an ADF1-like molecule comprising administering to said animal an effective amount of a nucleic acid sequence encoding ADF1-like molecule, ADF1 analog, biologically active ADF1-like fragment or ADF1-like fusion protein.

80. The method of claim 79 wherein said animal is a mammal.

81. The method of claim 79 wherein said animal is a human.

82. The method of claim 79 wherein said animal is a Drosophila species.

83. The method of claim 79 wherein the nucleic acid sequence is incorporated into a viral vector.

84. A method of treating an animal with a defect in synaptic plasticity associated with a defect in an ADF1-like molecule comprising administering to said animal a pharmaceutical agent which is capable of increasing expression of an Adf1-like gene in said animal, in an amount effective to increase expression of Adf1-like gene relative to expression of said Adf1-like gene in said animal prior to administration of said pharmaceutical agent, thereby resulting in treatment of said animal.

85. The method of claim 84 wherein said animal is a mammal.

86. The method of claim 84 wherein said animal is a human.

87. The method of claim 84 wherein said animal is a Drosophila species.

88. A method of treating an animal with a defect in synaptic plasticity associated with a defect in an ADF1-like molecule comprising administering to said animal a pharmaceutical agent which is capable of increasing functional ADF1-like activity in said animal, in an amount effective to increase the functional ADF1-like activity relative to the functional ADF1-like activity in said animal prior to administration of said pharmaceutical agent, thereby resulting in treatment of said animal.

89. The method of claim 88 wherein said animal is a mammal.

90. The method of claim 88 wherein said animal is a human.

91. The method of claim 88 wherein said animal is a Drosophila species.

92. A method of modulating synaptic plasticity in an animal comprising treating said animal to modulate expression of an Adf1-like gene.

93. The method of claim 92 wherein said animal is a mammal.

94. The method of claim 92 wherein said animal is a human.

95. The method of claim 92 wherein said animal is a Drosophila species.

96. A method of modulating synaptic plasticity in an animal comprising treating said animal to modulate functional ADF1-like activity.

97. The method of claim 96 wherein said animal is a mammal.

98. The method of claim 96 wherein said animal is a human.

99. The method of claim 96 wherein said animal is a Drosophila species.

100. A method of modulating synaptic plasticity in an animal comprising administering to said animal a pharmaceutical agent which is capable of modulating expression of an Adf1-like gene in said animal, in an amount effective to modulate expression of Adf1-like gene in said animal, thereby resulting in modulation of synaptic plasticity in said animal.

101. The method of claim 100 wherein said animal is a mammal.

102. The method of claim 100 wherein said animal is a human.

103. The method of claim 100 wherein said animal is a Drosophila species.

104. A method of modulating synaptic plasticity in an animal comprising administering to said animal a pharmaceutical agent which is capable of modulating functional ADF1-like activity in said animal, in an amount effective to modulate the functional ADF1-like activity in said animal, thereby resulting in modulation of synaptic plasticity in said animal.

105. The method of claim 104 wherein said animal is a mammal.

106. The method of claim 104 wherein said animal is a human.

107. The method of claim 104 wherein said animal is a Drosophila species.

108. A method of enhancing synaptic plasticity in an animal comprising treating said animal to increase expression of an Adf1-like gene relative to expression of said Adf1-like gene in said animal prior to said treatment.

109. The method of claim 108 wherein said animal is a mammal.

110. The method of claim 108 wherein said animal is a human.

111. The method of claim 108 wherein said animal is a Drosophila species.

112. A method of enhancing synaptic plasticity in an animal comprising treating said animal to increase functional ADF1-like activity relative to functional ADF1-like activity in said animal prior to said treatment.

113. The method of claim 112 wherein said animal is a mammal.

114. The method of claim 112 wherein said animal is a human.

115. The method of claim 112 wherein said animal is a Drosophila species.

116. A method of enhancing synaptic plasticity in an animal comprising administering to said animal an effective amount of ADF1-like molecule, ADF1 analog, biologically active ADF1-like fragment or ADF1-like fusion protein.

117. The method of claim 116 wherein said animal is a mammal.

118. The method of claim 116 wherein said animal is a human.

119. The method of claim 116 wherein said animal is a Drosophila species.

120. A method of enhancing synaptic plasticity in an animal comprising administering to said animal an effective amount of a nucleic acid sequence encoding ADF1-like molecule, ADF1 analog, biologically active ADF1-like fragment or ADF1-like fusion protein.

121. The method of claim 120 wherein said animal is a mammal.

122. The method of claim 120 wherein said animal is a human.

123. The method of claim 120 wherein said animal is a Drosophila species.

124. The method of claim 120 wherein the nucleic acid sequence is incorporated into a viral vector.

125. A method of enhancing synaptic plasticity in an animal comprising administering to said animal a pharmaceutical agent which is capable of increasing expression of an Adf1-like gene in said animal, in an amount effective to increase expression of Adf1-like gene relative to expression of said Adf1-like gene in said animal prior to administration of said pharmaceutical agent, thereby resulting in enhancement of synaptic plasticity in said animal.

126. The method of claim 125 wherein said animal is a mammal.

127. The method of claim 125 wherein said animal is a human.

128. The method of claim 125 wherein said animal is a Drosophila species.

129. A method of enhancing synaptic plasticity in an animal comprising administering to said animal a pharmaceutical agent which is capable of increasing functional ADF1-like activity in said animal, in an amount effective to increase the functional ADF1-like activity relative to the functional ADF1-like activity in said animal prior to administration of said pharmaceutical agent, thereby resulting in enhancement of synaptic plasticity in said animal.

130. The method of claim 129 wherein said animal is a mammal.

131. The method of claim 129 wherein said animal is a human.

132. The method of claim 129 wherein said animal is a Drosophila species.

133. A method of screening a pharmaceutical agent for its ability to modulate ADF1-like activity in an animal comprising the steps of:
a) administering said pharmaceutical agent to said animal; and
b) determining the functional ADF1-like activity in said animal obtained in step a) relative to the functional ADF1-like activity in a control animal to which said pharmaceutical has not been administered.

134. The method of claim 133 wherein said animal is an animal with a defect in long term memory formation associated with a defect in an ADF1-like molecule.

135. The method of claim 133 wherein said animal is a mammal.

136. The method of claim 133 wherein said animal is a Drosophila species.

137. A method of screening a pharmaceutical agent for its ability to modulate long term memory formation in an animal comprising the steps of:
a) administering said pharmaceutical agent to said animal;
b) determining the functional ADF1-like activity in said animal obtained in step a) relative to the functional ADF1-like activity in a control animal to which said pharmaceutical has not been administered;
c) selecting said animal in step b) having a functional ADF1-like activity which differs from the functional ADF1-like activity in the control animal to which said pharmaceutical agent has not been administered;
d) training said animal selected in step c) under conditions appropriate to produce long term memory formation in said animal;
e) assessing long term memory formation in said animal trained in step d); and
f) comparing long term memory formation assessed in step e) with long term memory formation produced in the control animal to which said pharmaceutical agent has not been administered.

138. The method of claim 137 wherein said animal is an animal with a defect in long term memory formation associated with a defect in an ADF1-like molecule.

139. The method of claim 137 wherein said animal is a mammal.

140. The method of claim 137 wherein said animal is a Drosophila species.

141. A method for assessing the effect of a pharmaceutical agent on ADF1-like activity in an animal comprising the steps of:
a) administering said pharmaceutical agent to an animal; and
b) determining the functional ADF1-like activity in said animal obtained in step a) relative to the functional ADF1-like activity in a control animal to which said pharmaceutical has not been administered.

142. The method of claim 141 wherein said animal is an animal with a defect in long term memory formation associated with a defect in an ADF1-like molecule.

143. The method of claim 141 wherein said animal is a mammal.

144. The method of claim 141 wherein said animal is a Drosophila species.

145. A method for assessing the effect a pharmaceutical agent on long term memory formation in an animal comprising the steps of:
a) administering said pharmaceutical agent to an animal;
b) determining the functional ADF1-like activity in said animal obtained in step a) relative to the functional ADF1-like activity in a control animal to which said pharmaceutical has not been administered;
c) selecting said animal in step b) having a functional ADF1-like activity which differs from the functional ADF1-like activity in the control animal to which said pharmaceutical agent has not been administered;
d) training said animal selected in step c) under conditions appropriate to produce long term memory formation in said animal;
e) assessing long term memory formation in said animal trained in step d); and
f) comparing long term memory formation assessed in step e) with long term memory formation produced in the control animal to which said pharmaceutical agent has not been administered.

146. The method of claim 145 wherein said animal is an animal with a defect in long term memory formation associated with a defect in an ADF1-like molecule.

147. The method of claim 145 wherein said animal is a mammal.

148. The method of claim 145 wherein said animal is a Drosophila species.

149. A method of screening for a pharmaceutical agent which is capable of modulating ADF1-like activity comprising the steps of:
a) introducing into host cells a DNA construct, wherein said DNA construct comprises (1) DNA encoding an indicator gene and (2) a promoter sequence comprising an ADF1 binding site, said promoter sequence operably linked to said DNA encoding said indicator gene;
b) producing a sample by introducing into host cells comprising said DNA construct a pharmaceutical agent to be assessed for its ability to modulate ADF1-like activity under conditions appropriate for expression of said indicator gene;
c) detecting expression of said indicator gene in said sample obtained in step b); and
d) comparing expression detected in step c) with expression of said indicator gene detected in control cells into which said pharmaceutical agent has not been introduced,
whereby a difference in expression of indicator gene in said sample obtained in step b) compared to the expression of indicator gene in control cells identifies the pharmaceutical agent as one which modulates ADF1-like activity.

150. The method of claim 149 wherein said host cells are cells of neural origin.

151. An antibody or antigen-binding fragment thereof which binds to ADF1.
